(19) United States
(12) Patent Application Publication
(10) Pub. No.: US 2015/0344502 A1
(43) Pub. Date: Dec. 3, 2015

(54) CEPHEM COMPOUND

(71) Applicant: SHIONOGI & CO., LTD., Osaka-shi, Osaka (JP)

(72) Inventors: Kenji YAMAWAKI, Toyonaka-shi, Osaka (JP); Hiroki KUSANO, Toyonaka-shi, Osaka (JP)

(21) Appl. No.: 14/655,893

(22) PCT Filed: Dec. 26, 2013
(86) PCT No.: PCT/JP2013/084782

§ 371 Date: Jun. 26, 2015

(30) Foreign Application Priority Data
Dec. 26, 2012 (JP) 2012-281970

(51) Int. Cl. C07D 519/06; C07D 501/46

(52) U.S. Cl. CPC C07D 519/06; C07D 501/46

(57) ABSTRACT

A compound of the formula:

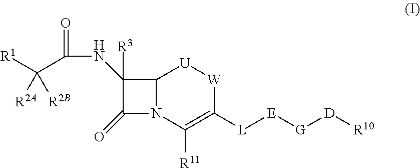

wherein, W is —CH2⁻ etc.; U is —S⁻ etc.;
R¹ is substituted or unsubstituted carbocyclyl etc.;
R^{2A} and R^{2B} is a) or b) described in the specification;
R³ is a hydrogen atom etc.; R¹¹ is carboxylate anion (—COO⁻) etc.;
L is substituted or unsubstituted lower alkylene etc.,
E is a substituted or unsubstituted divalent group containing quaternary ammonium ion;
G is a single bond etc.;
D is —C(═O)—C(═O)—NR⁶— etc.;
R¹⁰ 1) to 3) described in specification;
or an amino-protected compound when the amino group is present on the ring in the 7-side chain, or a pharmaceutically acceptable salt thereof.

### TECHNICAL FIELD

The compound of the subject invention are related to Cephem compounds, which have a wide antimicrobial spectrum, in particular exhibit potent antimicrobial activity against beta-lactamase producing Gram negative bacteria, and pharmaceutical compositions comprising the same.

### BACKGROUND ART

To date, a variety of beta-lactamase drugs have been developed and beta-lactam drugs have become clinically extremely important antimicrobial drugs. However, there are increasing the number of bacterial types which have obtained resistance against beta-lactam drugs by producing beta-lactamase, which degrade beta-lactam drugs.

According to the Ambler molecular classification, Beta-lactamases are largely classified into four classes. Specifically, there are Class A (TEM type, SHV type, CTX-M type, KPC type and the like), Class B (IMP type, VIM type, L-1 type and the like), Class C (AmpC type and the like) and Class D (OXA type and the like). Amongst these, Class A, C and D types are largely classified into serine-beta-lactamase, on the other hand, Class B type is classified into metallo-beta-lactamase. It has been known that both have respectively different mechanisms to each other in terms of hydrosis of beta-lactam drugs.

Recently, clinical problem has been occurring due to the existence of Gram negative bacteria which have become highly resistant to a number of beta-lactam drugs including Cephems and Carbapenems by producing Class A types (ESBL) which have an extended substrate spectrum, and Class D types serine-beta-lactamases, and Class B type metallo beta-lactamase. Particularly, metallo-beta-lactamase is known to be one of the causes of obtaining multidrug-resistance in Gram negative bacteria. Cephem compounds which exhibit intermediate activity against metallo-beta-lactamase producing Gram negative bacteria are known (e.g., Patent Document 1 and Non-patent Document 1). However, there is a demand for development of Cephem compounds which exhibit more potent antimicrobial activity, in particular more effective against a variety of beta-lactamase producing Gram negative bacteria.

One of the known antimicrobials having high anti-Gram negative bactericidal activity is Cephem compounds having a catechol group intramolecularly (e.g., Non-patent Documents 2-4). This action thereof is that the catechol group forms a chelate with Fe³⁺, thereby the compound is efficiently incorporated into the bacterial body through the Fe³⁺ dependent iron transport system (tonB-dependent iron transport system). Therefore, research has been conducted on compounds having catechol catechol or similar structure thereto, on the 3-side chain or 7-side chain moiety on the Cephem backbone.

Patent Documents 2-8 and Non-patent Document 5 describe compounds having a partial structure of the 7-side chain and a quaternary salt structure on the cephem backbone. However, these documents merely describe a pyridinium structure, and merely disclose compounds having a formamide group at the 7-position in most cases.

Non-patent Document 1 and Patent Documents 8˜12, 15 and 16 describe catechol type derivatives having a catechol group on the 3-side chain moiety on the Cephem backbone, Patent Document 10, 11, 13 and 14 describe pseudo-catechol type derivatives having a hydroxypyridone group on the 3-side chain moiety on the Cephem backbone. Patent Document 17 describe Cephem compound having a catechol group on the 3-side chain moiety on the Cephem backbone, but which do not have a quaternary ammonium group. Patent Documents 18-20, 23 and Non-patent Documents 8 and 9 disclose Cephem compounds having a quaternary ammonium group, but do not describe a catechol type derivative.

However, these documents do not describe the compounds of the subject invention. Moreover, in the above documents, which describe Cephem compounds having a catechol group in their structure, there is no description of Class B type metallo-beta-lactamase, and specific antimicrobial activity against a wide variety of Gram negative bacteria including Class B type.

Non-patent Document 7 describes that penicillin compounds having a tetrazolyl group at the 3-position of the penicillin skeleton have superior stability against beta-lactamase. However, a Cephem compound having a tetrazolyl group at the 4-position of the cephem skeleton is not disclosed in this document.

Patent Documents 18, 19, 20 and Non-patent Document 6 describe Cephem compounds having a tetrazolyl group at the 4-position of the cephem skeleton. However, a compound having a quaternary ammonium group at the 3-side chain is not disclosed in these documents.

Moreover, the present applicant filed an application of Cephem compounds having catechol type substituents (Patent Documents 21 to 26). However, these documents do not describe the compound of the subject invention.

### PRIOR ART DOCUMENT

### Patent Document

### [Patent Document 1]

International Publication No. 2007/119511 pamphlet

### [Patent Document 2]

Japanese Laid-Open Publication No. 57-118588

### [Patent Document 3]

European Patent Application Publication No. 114752

### [Patent Document 4]

European Patent Application Publication No. 168177

### [Patent Document 5]

European Patent Application Publication No. 211656

### [Patent Document 6]

European Patent Application Publication No. 305111

### [Patent Document 7]

Japanese Patent Application Publication No. 4-364189

### [Patent Document 8]

Japanese Patent Application Publication No. 3-173893

### [Patent Document 9]

Japanese Patent Application Publication No. 2-15090

### [Patent Document 10]

Japanese Patent Application Publication No. 2-28187

### [Patent Document 11]

Japanese Patent Application Publication No. 2-117678

### [Patent Document 12]

Japanese Patent Application Publication No. 6-510523

### [Patent Document 13]

Japanese Patent Application Publication No. 5-213971

### [Patent Document 14]

Japanese Patent Application Publication No. 2-28185

### [Patent Document 15]

International Publication No. 2007/096740 pamphlet

### [Patent Document 16]

Japanese Patent Application Publication No. 62-30788

### [Patent Document 17]

Japanese Patent Application Publication No. 5-59065

### [Patent Document 18]

European Patent Application Publication No. 366189

### [Patent Document 19]

European Patent Application Publication No. 335297

### [Patent Document 20]

Japanese Patent Application Publication No. 63-10792

### [Patent Document 21]

International Publication No. 2010/050468

### [Patent Document 22]

International Publication No. 2011/125966

### [Patent Document 23]

International Publication No. 2011/125967

### [Patent Document 24]

International Publication No. 2011/136268

### [Patent Document 25]

International Publication No. 2012/147773

### [Patent Document 26]

International Publication No. 2013/052568

### Non-Patent Document

### [Non-Patent Document 1]

Applied Microbiology and Biotechnology (1994), 40(6), 892-7

### [Non-Patent Document 2]

The Journal of Antibiotics, vol. 46, pp. 833-839 (1993)

### [Non-Patent Document 3]

The Journal of Antibiotics, vol. 43, pp. 1617-1620 (1990)

### [Non-Patent Document 4]

The Journal of Antibiotics, vol. 42, pp. 795-806 (1989)

### [Non-Patent Document 5]

The Journal of Antibiotics, vol. 40, pp. 646-651 (1987)

### [Non-Patent Document 6]

American Review of Respiratory Disease (1990), 141(3), 672-7

### [Non-Patent Document 7]

The Journal of Antibiotics, vol. 39, pp. 76-89 (1986)

### [Non-Patent Document 8]

Acta Pharmaceutica, vol. 50, pp. 17-27 (2000)

### [Non-Patent Document 9]

Journal of Medicinal Chemistry, vol. 34, pp. 669-675 (1991)

### SUMMARY OF THE INVENTION

### Problems to be Solved by the Invention

The subject invention provides Cephem compounds which exhibit potent antimicrobial spectrum against a variety of bacteria including Gram negative bacteria and/or Gram positive bacteria. Preferably, the subject invention provides Cephem compounds which exhibit potent antimicrobial activity against beta-lactamase producing Gram negative bacteria. More preferably, the subject invention provides Cephem compounds which exhibit potent antimicrobial activity against multidrug-resistant bacteria, in particular, Class B type metallo-beta-lactamase producing Gram negative bacteria. Still more preferably, the subject invention provides Cephem compounds which exhibit effective antimicrobial activity against extended-spectrum beta-lactamase (ESBL) producing bacteria.

### Means for Solving the Problem

The subject invention provides Cephem compounds which have solved the above-mentioned problems by having the following characteristics in structure:

The compound has a quaternary ammonium group represented as E at the 3-side chain of the cephem. E is preferably a cyclic structure.

The compound has a group represented as D at the 3-side chain of the cephem. D is preferably a spacer such as carbonyl, amide, ester and the like.

The compound has a cyclic structure represented as R¹⁰ at the 3-side chain of the cephem. R¹⁰ preferably has a catechol structure.

The subject invention specifically provides the following invention.

### (Item 1)

A compound of Formula (I):

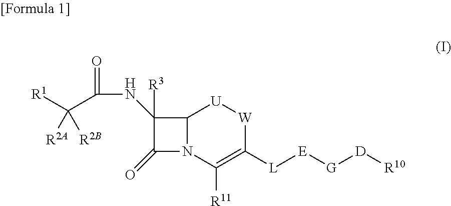

or an amino-protected compound when the amino group is present on the ring in the 7-side chain, or a pharmaceutically acceptable salt thereof,
wherein

W is —CH₂—,—S— or —O—,

a) U is —CH₂—, —S—, —S(═O)— or—O—, when W is —CH₂—; or,

b) U is —CH₂—, when W is —S— or —O—;

L is substituted or unsubstituted lower alkylene or substituted or unsubstituted lower alkenylene;

R¹ is substituted or unsubstituted carbocyclyl or substituted or unsubstituted heterocyclyl;

with regard to R^{2A} and R^{2B},

a) R^{2A} is a hydrogen atom, substituted or unsubstituted amino, —SO₃H, substituted or unsubstituted amino sulfonyl, carboxy, substituted or unsubstituted lower alkyloxycarbonyl, substituted or unsubstituted carbamoyl, hydroxy, or substituted carbonyloxy; and R^{2B} is a hydrogen atom, or

b) R^{2A} and R^{2B} are taken together to form substituted or unsubstituted methylidene, or substituted or unsubstituted hydroxyimino;

R³ is a hydrogen atom, —OCH₃ or —NH—CH(═O);

R¹ is carboxylate anion (—COO⁻) or a bioisoster of carboxylate anion (—COO⁻);

E is a substituted or unsubstituted divalent group containing quaternary ammonium ion(s);

G is a single bond, substituted or unsubstituted lower alkylene, substituted or unsubstituted lower alkenylene, or substituted or unsubstituted lower alkynylene;

D is a single bond, —C(═O)—, —O—C(═O)—, —C(═O)—O—, —NR⁶—C(═O)—, —C(═O)—NR⁶—, —C(═O)—C(═O)—NR⁶—, —NR⁶—C(═O)—C(═O)—, —C(═O)—NR⁶—C(═O)—, —C(═O)—C(═O)—, —O—, —NR⁶—NR⁶—C(═O)—, —C(═O)—NR⁶—NR⁶—, —N═N—C(═O)—, —C(═O)—N═N—, —C═N—NR⁶—C(═O)—, —C═N—C(═O)—, —N═CR⁶—C(═O)—, —C═N—C(═O)—NR⁶—, —NR⁶—C(═O)—C(═N—OR⁶)—, —C(═N—OR⁶)—C(═O)—NR⁶—, —NR⁶—C(═N—OR⁶)—, —C(═N—OR⁶)—NR⁶—, —C(═O)—C(═N—OR⁶)—, —C(═N—OR⁶)—C(═O)—, —S—, —S(═O)—, —S(═O)₂—NR⁶—, —NR⁶—S(═O)₂—, —NR⁶—CH₂—, —CH₂—NR⁶—, —S(═O)₂— or —NR⁶—;

R⁶ is independently a hydrogen atom or substituted or unsubstituted lower alkyl;

R¹⁰ is

1) substituted or unsubstituted phenyl or substituted or unsubstituted 6-membered heterocyclyl containing 1-3 nitrogen atoms;

2) substituted or unsubstituted 9-membered bicyclic aromatic heterocyclyl; or

3) a group represented by Formula

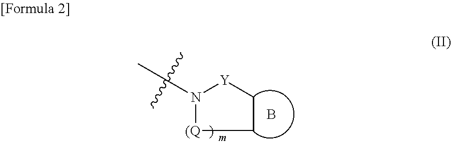

wherein

ring B is substituted or unsubstituted carbocycle or substituted or unsubstituted heterocycle;

Y is —C(═O)— or —S(═O)₂—;

Q is independently —O—, —S—, —NR⁸—, —CR⁸R⁹—, —C(═O)—, —S(═O)₂— or —N═CH—;

R⁸ and R⁹ are each independently a hydrogen atom or substituted or unsubstituted lower alkyl;

m is an integer from 1 to 3,

provided that,

a) D is —C(═O)—C(═O)—NR⁶—, —NR⁶—C(═O)—C(═O)—, —C(═O)—NR⁶—C(═O)—, —NR⁶—NR⁶—C(═O)—, —C(═O)—NR⁶—NR⁶—, —N═N—C(═O)—, —C(═O)—N═N—, —C═N—NR⁶—C(═O)—, —C═N—C(═O)—, —N═CR⁶—C(═O)—, —C═N—C(═O)—NR⁶—, —NR⁶—C(═O)—C(═N—OR⁶)—, —C(═N—OR⁶)—C(═O)—NR⁶—, —NR⁶—C(═N—OR⁶)—, —C(═N—OR⁶)—NR⁶, —C(═O)—C(═N—OR⁶)—, —C(═N—OR⁶)—C(═O)—, or —C(═O)—C(═O)—, when R¹⁰ is the above 1);

b) R¹⁰ is not the group represented by Formula:

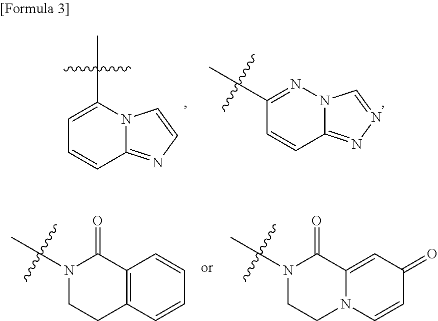 [SMILES: *c1cccc2nccn12 ; *c1ccc2nncn2n1 ; *N1CCc2ccccc2C1=O ; *N1CCn2ccc(=O)cc2C1=O]

wherein the above ring is optionally substituted by one or more substituents selected from hydroxy, chloro, fluoro, bromo, carboxy and methoxy; and
c) the compounds
wherein
R¹⁰ is the above 2) or 3),
E is the group represented by Formula:

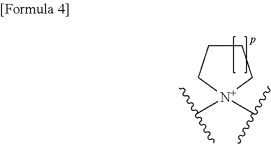

wherein p is an integer form 1 to 3, and
D is —NR⁶—C(═O)—, are excluded.

### (Item 2)

The compound, or an amino-protected compound when the amino is present on the ring in the 7-side chain, or a pharmaceutically acceptable salt thereof according to Item 1, wherein D is a single bond, —C(═O)—, —O—C(═O)—, —C(═O)—O—, —C(═O)—C(═O)—NR⁶—, —NR⁶—C(═O)—C(═O)—, —C(═O)—NR⁶—C(═O)—, —C(═O)—C(═O)—, —O—, —NR⁶—NR⁶—C(═O)—, —C(═O)—NR⁶—NR⁶—, —N═N—C(═O)—, —C(═O)—N═N—, —C═N—NR—C(═O)—, —C═N—C(═O)—, —N═CR⁶—C(═O)—, —C═N—C(═O)—NR⁶—, —NR⁶—C(═O)—C(═N—OR⁶)—, —C(═N—OR⁶)—C(═O)—NR⁶—, —NR⁶—C(═N—OR⁶)—, —C(═N—OR⁶)—NR⁶—, —C(═O)—C(═N—OR⁶)—, —C(═N—OR⁶)—C(═O)—, —S—, —S(═O)—, —S(═O)₂—NR⁶—, —NR⁶—S(═O)₂—, —NR⁶—CH₂—, —CH₂—NR₆—, —S(═O)₂—, or —NR⁶—.

### (Item 3)

The compound, or an amino-protected compound when the amino is present on the ring in the 7-side chain, or a pharmaceutically acceptable salt thereof according to Item 1, wherein R¹⁰ is the above 1), and D is —C(═O)—C(═O)—NR⁶—, —NR⁶—C(═O)—C(═O)—, —C(═O)—NR⁶—C(═O)—, —NR⁶—NR⁶—C(═O)—, —C(═O)—NR⁶—NR⁶—, —N═N—C(═O)—, —C(═O)—N═N—, —C═N—NR⁶—C(═O)—, —C═N—C(═O)—, —N═CR⁶—C(═O)—, —C═N—C(═O)—NR⁶—, —NR⁶—C(═O)—C(═N—OR⁶)—, —C(═N—OR⁶)—C(═O)—NR⁶—, —NR⁶—C(═N—OR⁶)—, —C(═N—OR⁶)—NR⁶—, —C(═O)—C(═N—OR⁶)—, —C(═N—OR⁶)—C(═O)—, or —C(═O)—C(═O)—.

### (Item 4)

The compound, or an amino-protected compound when the amino is present on the ring in the 7-side chain, or a pharmaceutically acceptable salt thereof according to Item 3, wherein D-R¹⁰ is the group represented by Formula:

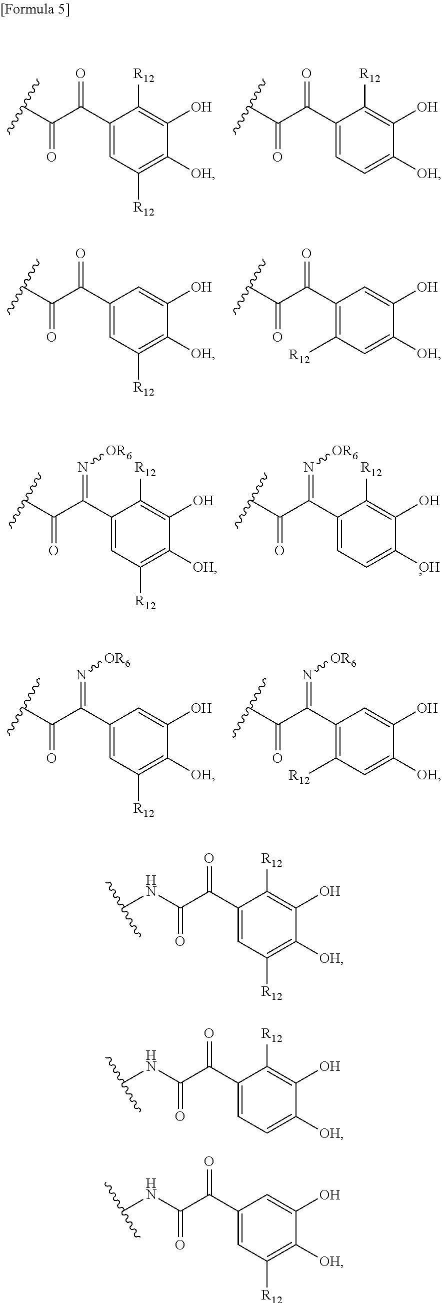

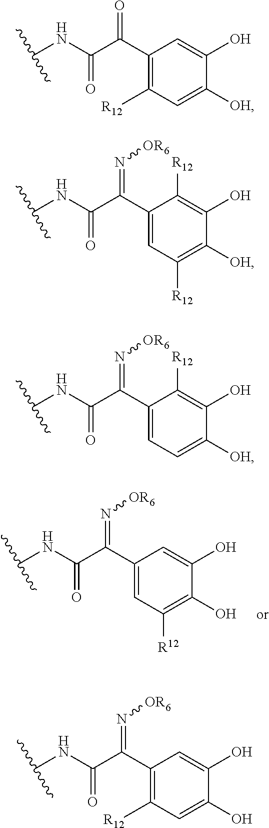

wherein

each R¹² is independently a hydrogen atom, halogen, hydroxy, —CN, —C(═O)—R¹⁵, —C(═O)OH, C(═O)—OR¹⁵ or OR¹⁵,

each R¹⁵ is independently lower alkyl or halo lower alkyl,

each R⁶ is independently a hydrogen atom or substituted or unsubstituted lower alkyl,

the bond of wavy line means that the bond is cis-configuration, trans-configuration or the mixture thereof.

### (Item 5)

The compound, or an amino-protected compound when the amino is present on the ring in the 7-side chain, or a pharmaceutically acceptable salt thereof according to Item 3, wherein D-R¹⁰ is the group represented by Formula:

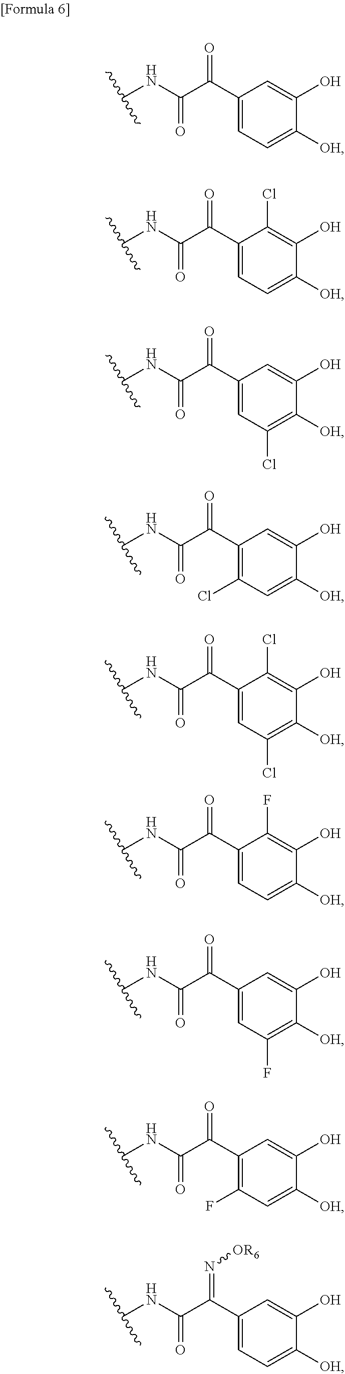

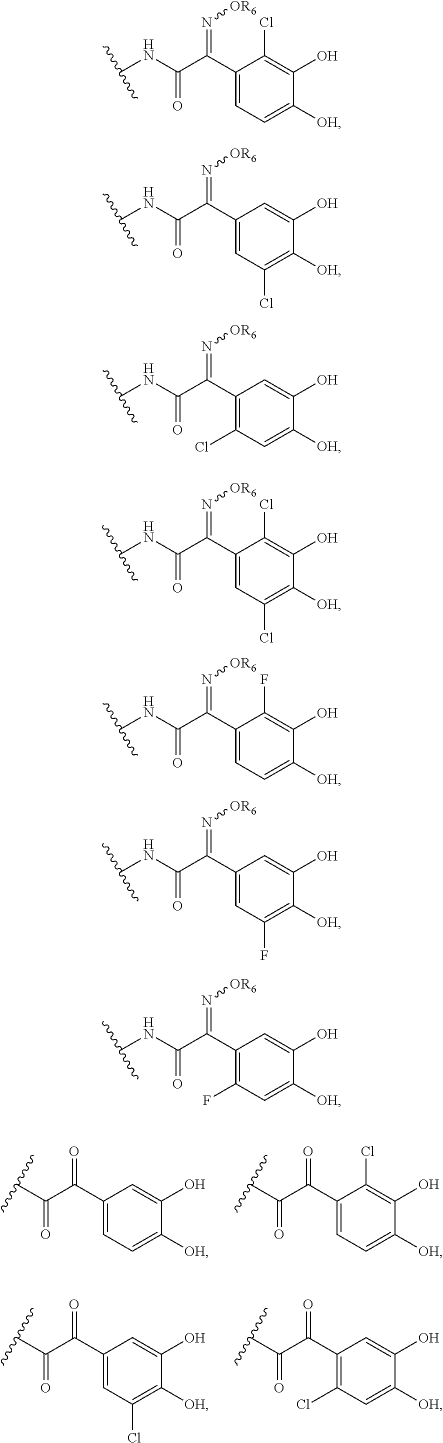

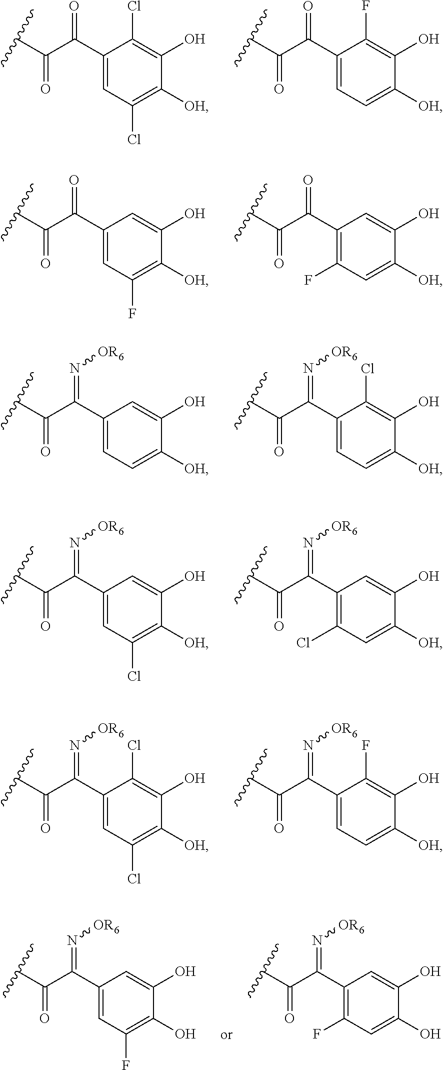

wherein

each R⁶ is independently a hydrogen atom, methyl, ethyl, 1-carboxy ethyl or 2-carboxy propane-2-yl,

the bond of wavy line means that the bond is cis-configuration, trans-configuration or the mixture thereof.

### (Item 6)

The compound, or an amino-protected compound when the amino is present on the ring in the 7-side chain, or a pharmaceutically acceptable salt thereof according to Item 1 or 2, wherein R¹⁰ is the above 2).

### (Item 7)

The compound, or an amino-protected compound when the amino is present on the ring in the 7-side chain, or a pharmaceutically acceptable salt thereof according to Item 6, wherein D-R¹⁰ is the group represented by Formula:

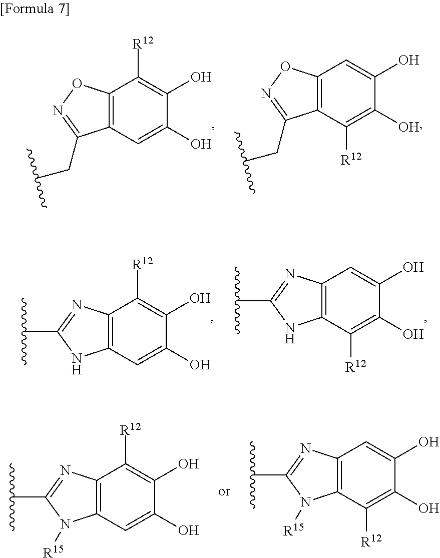

wherein

each R¹² is independently a hydrogen atom, halogen, hydroxy, —CN, —C(═O)—R¹⁵, —C(═O)—OH, —C(═O)—OR¹⁵ or OR¹⁵,

each R¹⁵ is independently lower alkyl or halo lower alkyl.

### (Item 8)

The compound, or an amino-protected compound when the amino is present on the ring in the 7-side chain, or a pharmaceutically acceptable salt thereof according to Item 6, wherein D-R¹⁰ is the group represented by Formula:

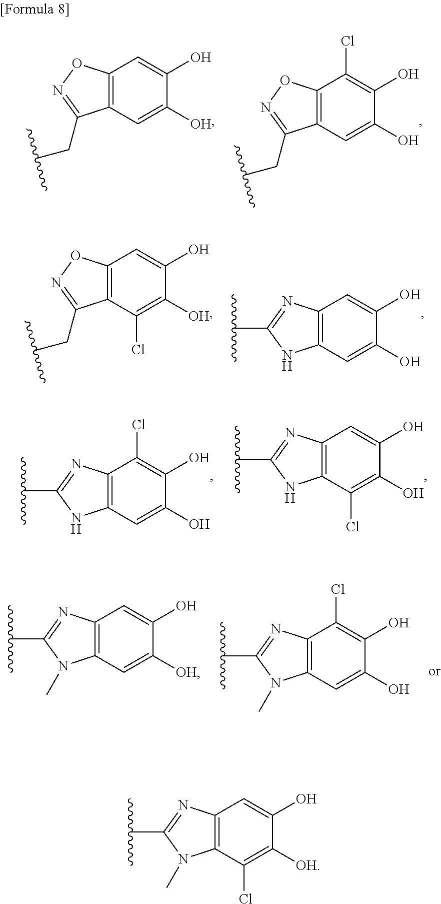

### (Item 9)

The compound, or an amino-protected compound when the amino is present on the ring in the 7-side chain, or a pharmaceutically acceptable salt thereof according to Item 1 or 2, wherein R¹⁰ is the above 3.

### (Item 10)

The compound, or an amino-protected compound when the amino is present on the ring in the 7-side chain, or a pharmaceutically acceptable salt thereof according to any one of Items 1, 2 and 9, wherein ring B is substituted or unsubstituted carbocycle.

### (Item 11)

The compound, or an amino-protected compound when the amino is present on the ring in the 7-side chain, or a pharmaceutically acceptable salt thereof according to any one of Items 1, 2, 9 and 10, wherein Y is —C(═O)—.

### (Item 12)

The compound, or an amino-protected compound when the amino is present on the ring in the 7-side chain, or a pharmaceutically acceptable salt thereof according to any one of Items 1, 2, 9, 10 and 11, wherein

m is 1 or 2;

Q is —O—, —S—, —NR⁸—, —CR⁸R⁹—, —C(═O)— or —N═CH—, when m is 1;

each Q is independently —O—, —S—, —NR⁸—, or —C(═O)—, when m is 2.

### (Item 13)

The compound, or an amino-protected compound when the amino is present on the ring in the 7-side chain, or a pharmaceutically acceptable salt thereof according to Item 9, wherein D-R¹⁰ is the group represented by Formula

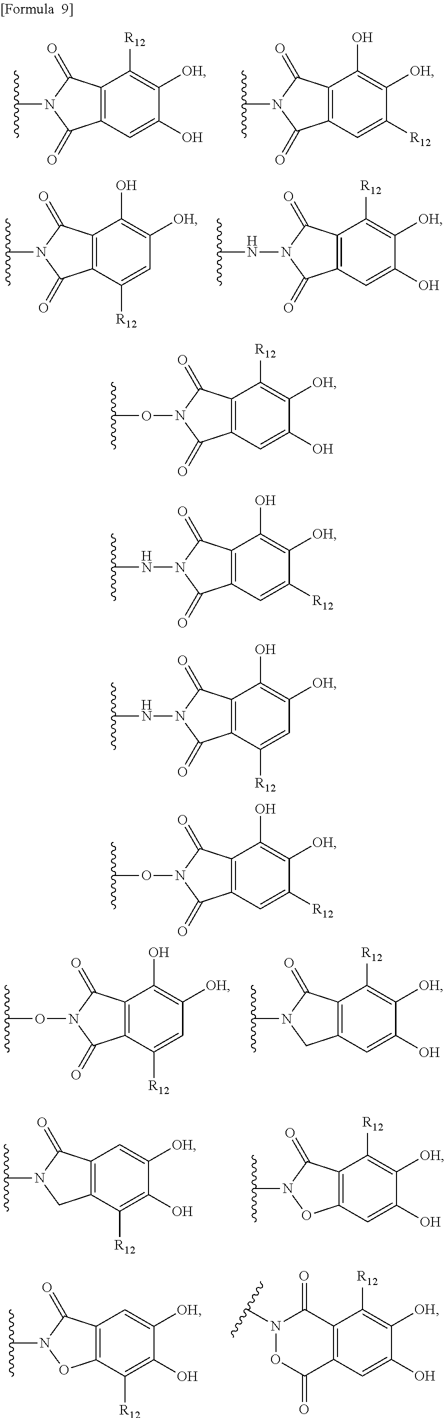

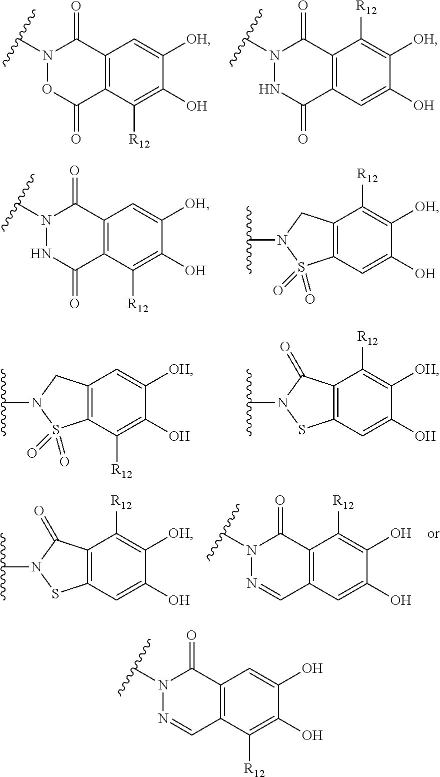

wherein

each R¹² is independently a hydrogen atom, halogen, hydroxy, —CN, —C(═O)—R¹⁵, —C(═O)—OH, —C(═O)—OR¹⁵ or OR¹⁵,

each R¹⁵ is independently lower alkyl or halo lower alkyl.

### (Item 14)

The compound, or an amino-protected compound when the amino is present on the ring in the 7-side chain, or a pharmaceutically acceptable salt thereof according to Item 9, wherein D-R¹⁰ is the group represented by Formula:

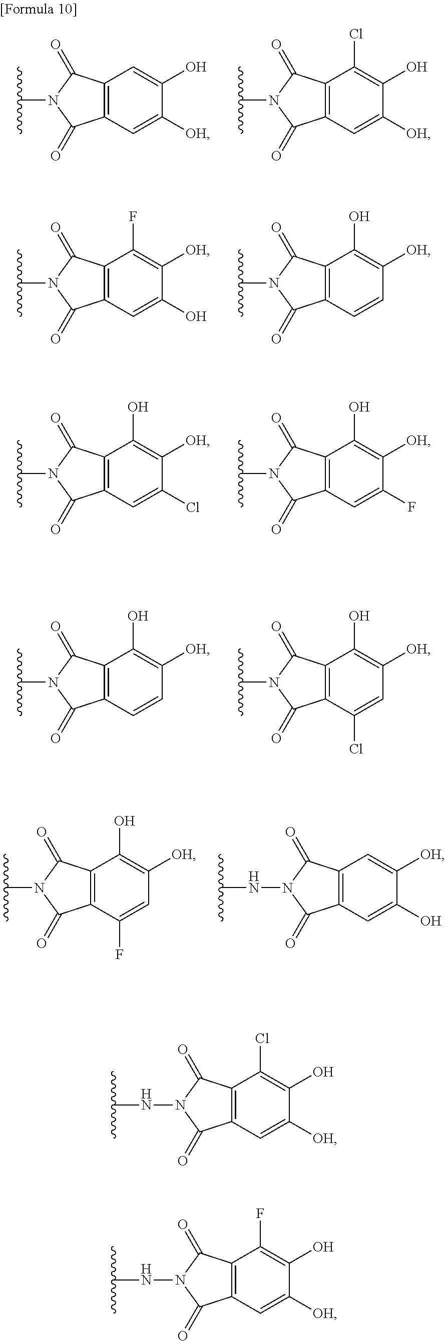

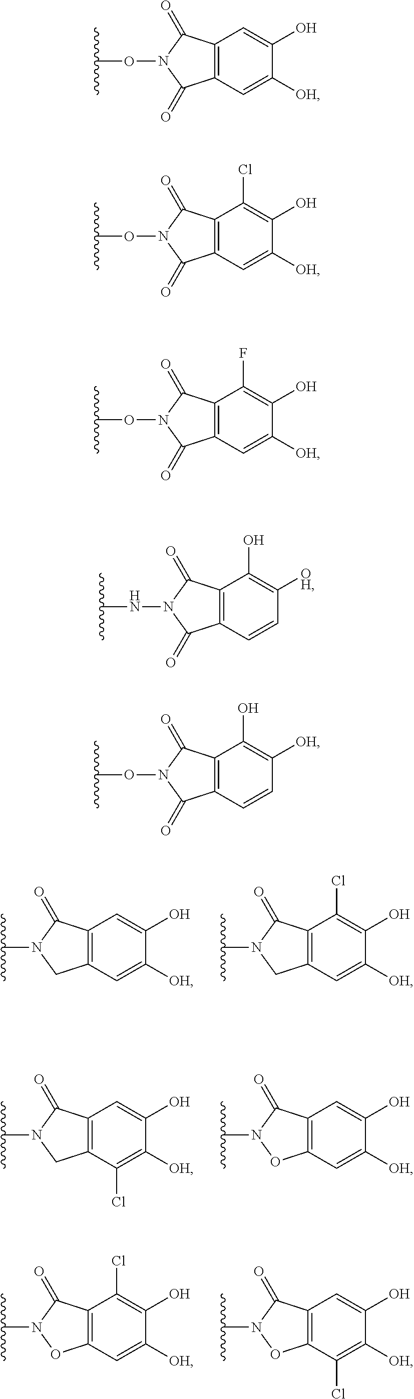

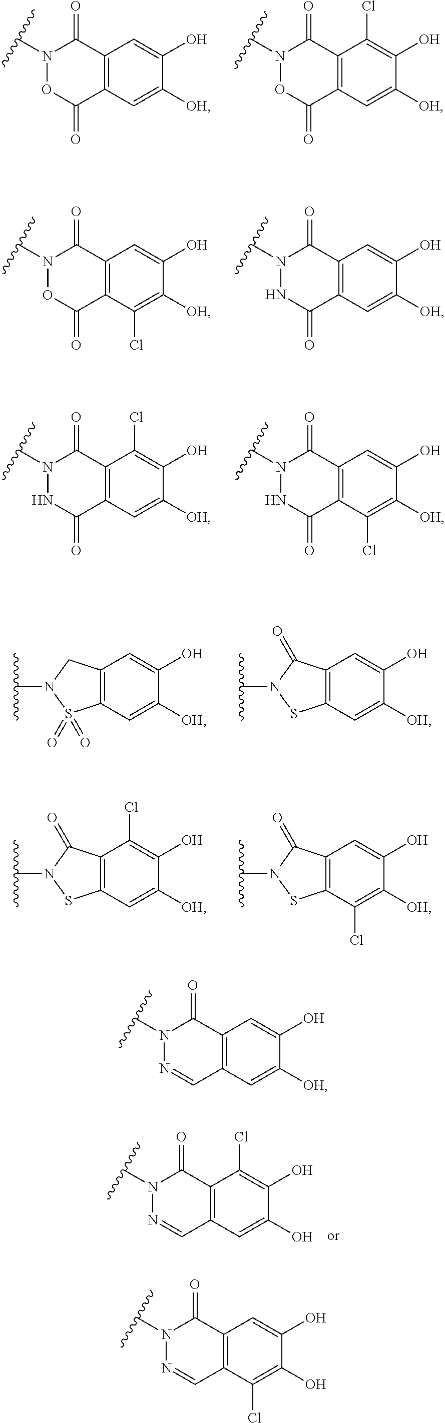

### (Item 15)

The compound, or an amino-protected compound when the amino is present on the ring in the 7-side chain, or a pharmaceutically acceptable salt thereof according to any one of Items 1 to 14, wherein E is substituted or unsubstituted, saturated or unsaturated, monocyclic or polycyclic divalent group containing a quaternary ammonium ion represented by Formula:

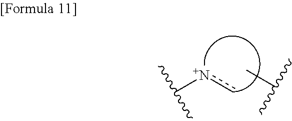

wherein

the dashed line is a bond in the ring,

the bond attached to the cationic nitrogen atom binds to L, and the other bond binds to G;

provided that,

when G binds to a cationic nitrogen atom, the dashed line is absent, and

when G does not bind to a cationic nitrogen atom, the dashed line is a single bond between the cationic nitrogen atom and a neighboring atom or an alkylene connecting the cationic nitrogen atom to a ring member atom other than said neighboring atom.

### (Item 16)

The compound, or an amino-protected compound when the amino is present on the ring in the 7-side chain, or a pharmaceutically acceptable salt thereof according to any one of Items 1 to 14, wherein E is substituted or unsubstituted, saturated or unsaturated, monocyclic or polycyclic divalent group containing a quaternary ammonium ion represented by Formula

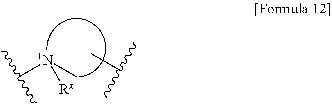

wherein
the bond attached to the cationic nitrogen atom binds to L, and the other bond binds to G;
R^{x} is substituted or unsubstituted lower alkyl.

### (Item 17)

The compound, or an amino-protected compound when the amino is present on the ring in the 7-side chain, or a pharmaceutically acceptable salt thereof according to any one of Items 1 to 14, wherein E is a group selected from the following formulae which are optionally substituted on the ring:

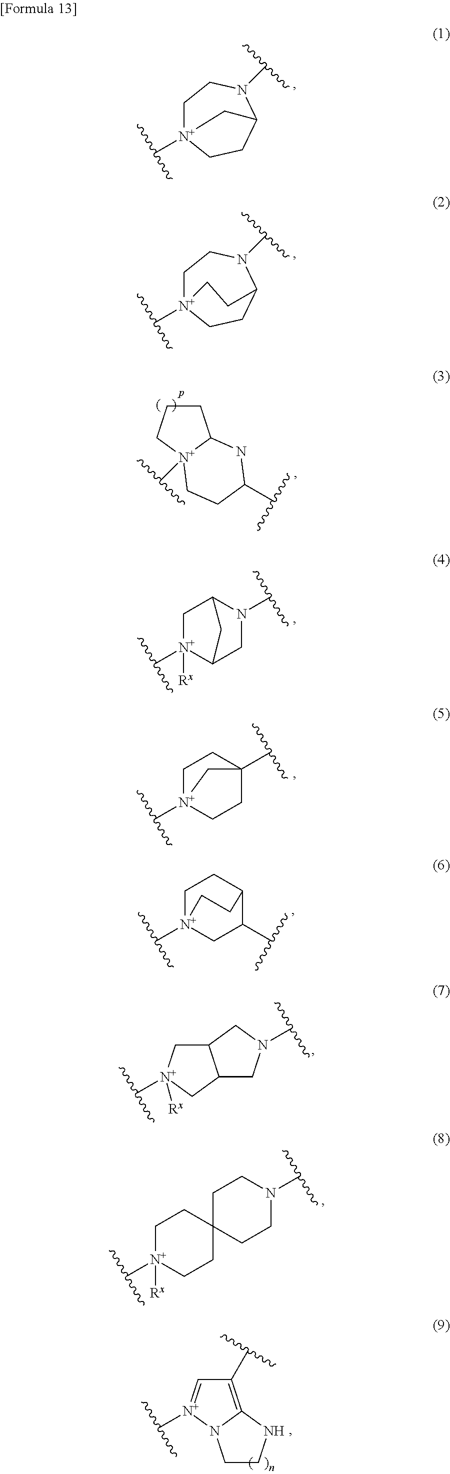

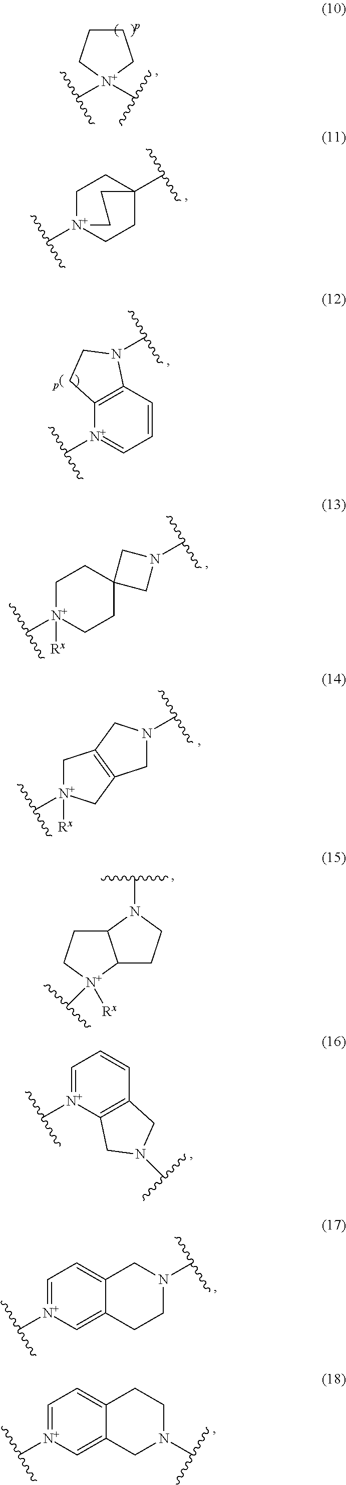

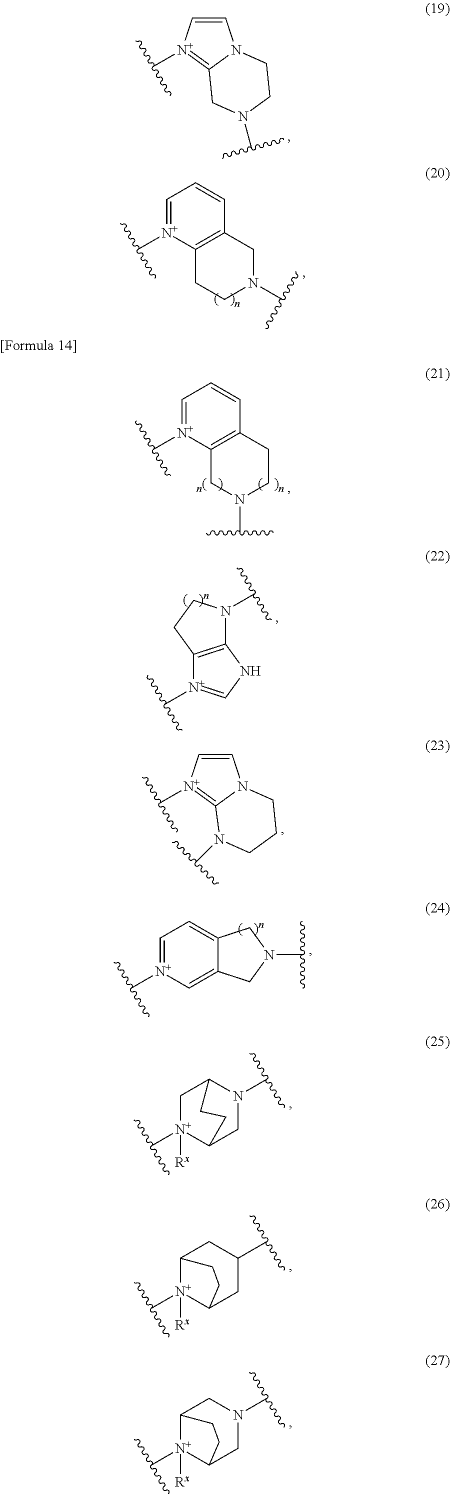

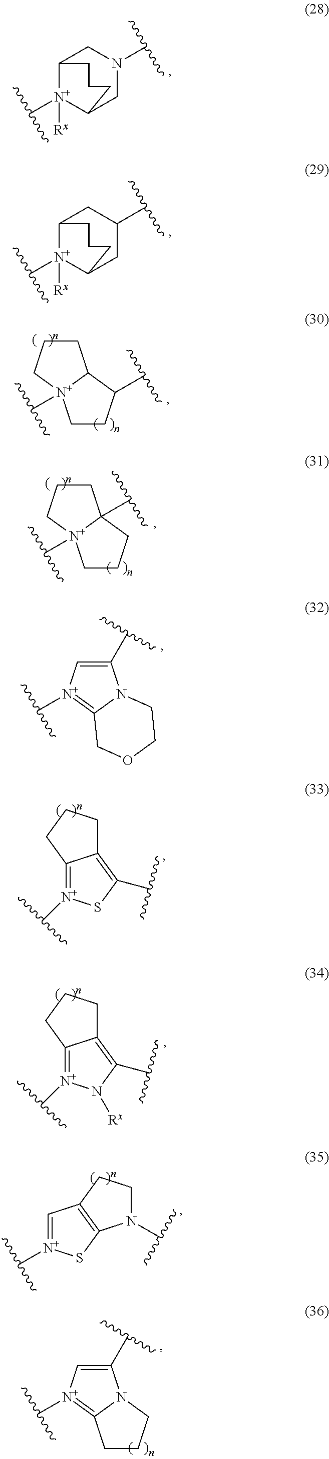

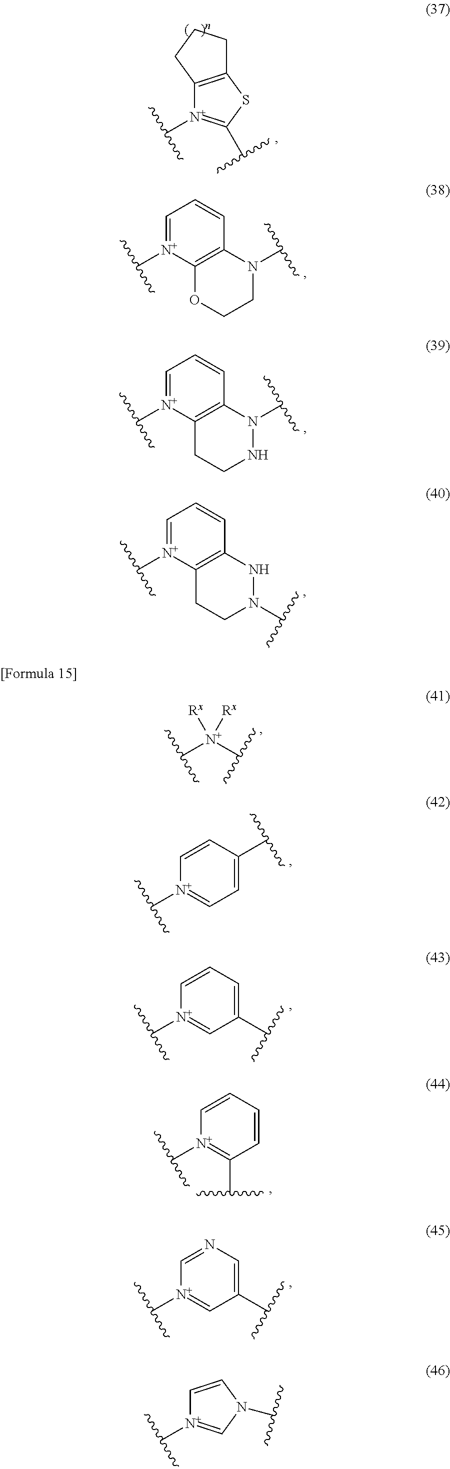

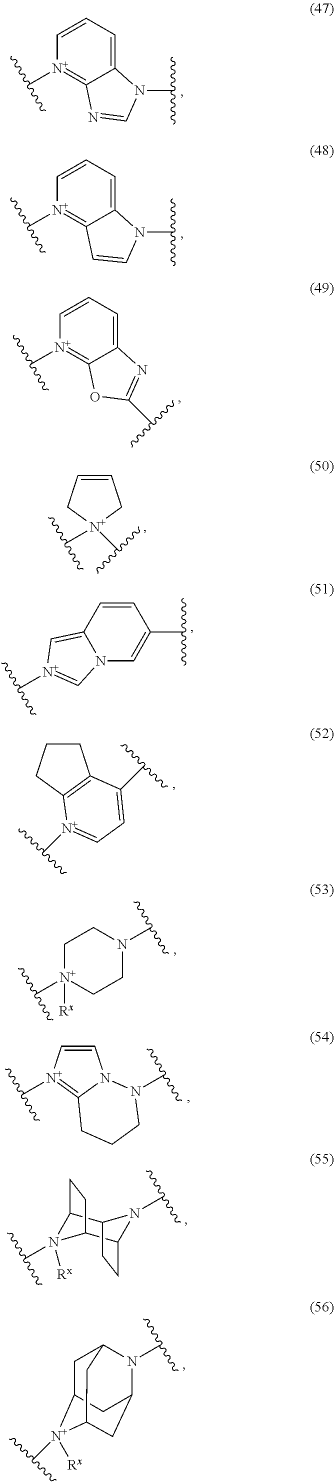

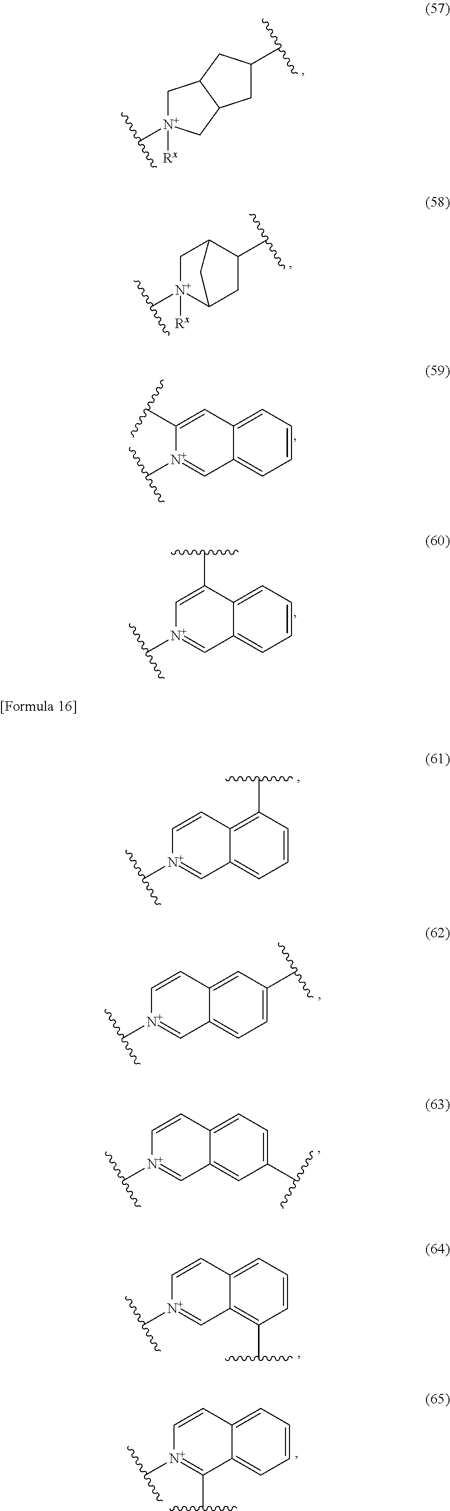

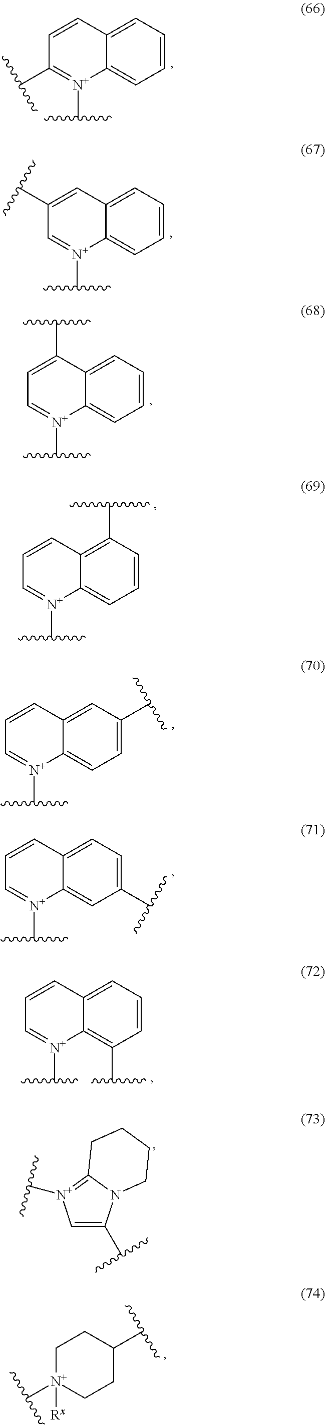

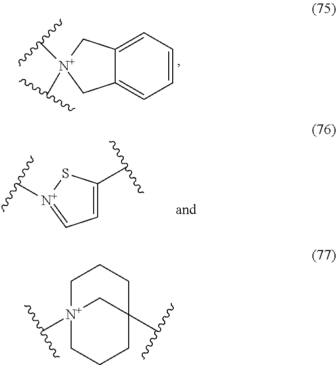 [SMILES: *[N+]1(*)Cc2ccccc2C1 ; *c1cc[n+](*)s1 ; *C12CCC[N+](*)(CCC1)C2]

wherein

the bond attached to the cationic nitrogen atom binds to L, and the other bond binds to G;
p is an integer from 1 to 3; n is 1 or 2; each R^{x} is independently substituted or unsubstituted lower alkyl.

### (Item 18)

The compound, or an amino-protected compound when the amino is present on the ring in the 7-side chain, or a pharmaceutically acceptable salt thereof according to Item 17, wherein E is a group selected from the group consisting of Formulae (1), (2), (5), (7), (10), (11), (26) to (29), (31) and (41).

### (Item 19)

The compound, or an amino-protected compound when the amino is present on the ring in the 7-side chain, or a pharmaceutically acceptable salt thereof according to any one of Items 1 to 18, wherein the bioisostere of carboxylate anion (—COO⁻) is selected from —SO₃⁻, —S(═O)₂—N⁻—R¹³, —PO⁻—(OR¹³), —PO₂⁻—(OR¹³), —N⁻—C(═O)—R¹³, —C(═O)—N⁻—OR¹³, —C(═O)—NH—N⁻—S(═O)₂—R¹³, —C(═O)—N⁻—S(═O)₂—R¹³, —C(═O)—CH═C(O⁻)—R¹³, —N⁻—S(═)₂—R¹³, —C(═O)—N⁻—S(═O)₂—R¹³, —N⁻—S(═O)₂—R¹³, —C(═O)—N⁻—C(═O)—R¹³, —C(═O)—N⁻—S(═O)₂—R¹³, —N⁻—C(═O)—R¹³,

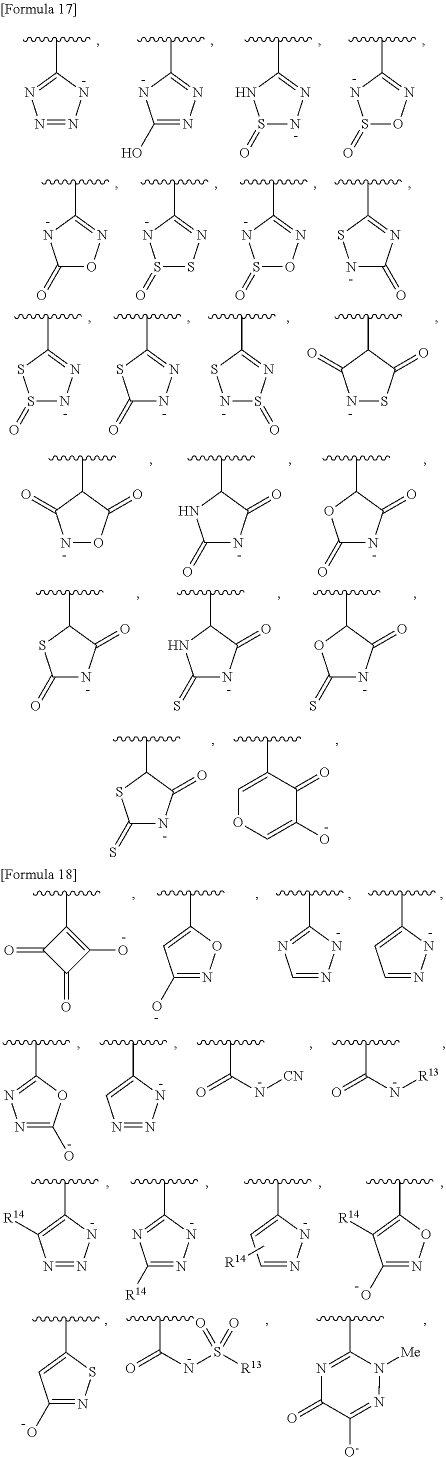

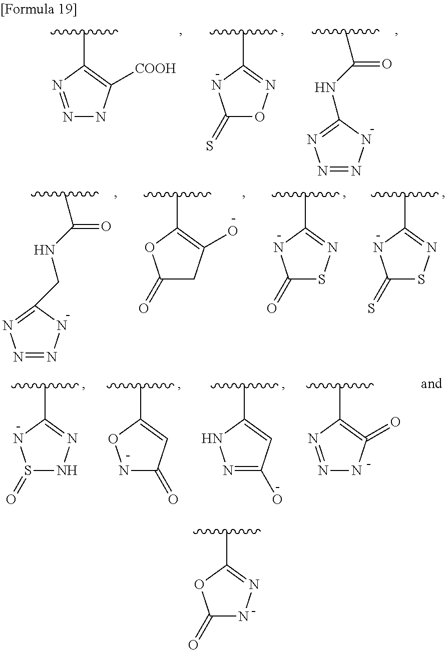

wherein

R¹³ is selected from the group consisting of hydrogen, hydroxy, halogen, substituted or unsubstituted lower alkyl, substituted or unsubstituted lower alkenyl, substituted or unsubstituted lower alkynyl, substituted or unsubstituted lower alkyloxy, substituted or unsubstituted amino, lower alkenyloxy, substituted or unsubstituted aryloxy, cyano, nitro, imino, mercapto, lower alkylthio, lower alkylsulfonyl, substituted or unsubstituted carbocyclyl, substituted or unsubstituted heterocyclyl and —CO₂R¹⁷ wherein R¹⁷ is hydrogen, lower alkyl or lower alkenyl; R¹⁴ is an electron-withdrawing group.

### (Item 20)

The compound, or an amino-protected compound when the amino is present on the ring in the 7-side chain, or a pharmaceutically acceptable salt thereof according to Item 19, wherein the bioisoster of carboxylate anion (—COO⁻) is the group represented by Formula:

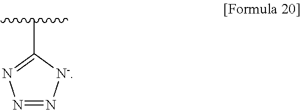

### (Item 21)

The compound, or an amino-protected compound when the amino is present on the ring in the 7-side chain, or a pharmaceutically acceptable salt thereof according to any one of Items 1 to 20, wherein U is —S—.

### (Item 22)

The compound, or an amino-protected compound when the amino is present on the ring in the 7-side chain, or a pharmaceutically acceptable salt thereof according to any one of Items 1 to 21, wherein W is —CH₂—.

### (Item 23)

The compound, or an amino-protected compound when the amino is present on the ring in the 7-side chain, or a pharmaceutically acceptable salt thereof according to any one of Items 1 to 22, wherein R³ is a hydrogen atom or —OCH₃.

### (Item 24)

The compound, or an amino-protected compound when the amino is present on the ring in the 7-side chain, or a pharmaceutically acceptable salt thereof according to any one of Items 1 to 23, wherein L is —CH₂—, —CH═CH—, —CH₂—CH═CH— or —CH═CH—CH₂—.

### (Item 25)

The compound, or an amino-protected compound when the amino is present on the ring in the 7-side chain, or a pharmaceutically acceptable salt thereof according to any one of Items 1 to 24, wherein G is a single bond or substituted or unsubstituted lower alkylene.

### (Item 26)

The compound, or an amino-protected compound when the amino is present on the ring in the 7-side chain, or a pharmaceutically acceptable salt thereof according to any one of Items 1 to 25, wherein R¹ is represented by Formula

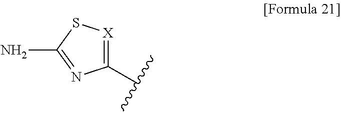

wherein X is N, C(—H) or C(—Cl).

### (Item 27)

The compound, or an amino-protected compound when the amino is present on the ring in the 7-side chain, or a pharmaceutically acceptable salt thereof according to Item 26, wherein X is N.

### (Item 28)

The compound, or an amino-protected compound when the amino is present on the ring in the 7-side chain, or a pharmaceutically acceptable salt thereof according to Item 26, wherein X is C(—H) or C(—Cl).

### (Item 29)

The compound, or an amino-protected compound when the amino is present on the ring in the 7-side chain, or a pharmaceutically acceptable salt thereof according to any one of Items 1 to 28, wherein R^{2A} and R^{2B} are taken together to form a substituted methylidene group shown below:

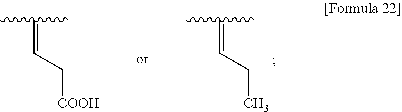

or
a substituted hydroxy imino group shown below:

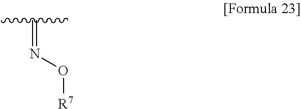

wherein R⁷ is substituted or unsubstituted lower alkyl group.

### (Item 30)

The compound, or an amino-protected compound when the amino is present on the ring in the 7-side chain, or a pharmaceutically acceptable salt thereof according to any one of Items 1 to 28, wherein R^{2A} and R^{2B} are taken together to form a substituted hydroxyimino group shown below:

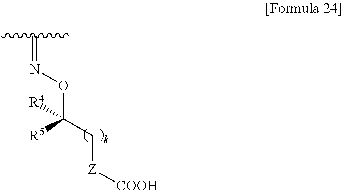

wherein

R⁴ and R⁵ are each independently a hydrogen atom, halogen, hydroxy, a carboxy group, a substituted or unsubstituted lower alkyl group, substituted or unsubstituted carbocyclyl, or substituted or unsubstituted heterocyclyl, or R⁴ and R⁵ may be taken together with a neighboring atom to form substituted or unsubstituted carbocycle or substituted or unsubstituted heterocycle;

Z is a single bond, optionally substituted carbocycle, or optionally substituted heterocycle;

k is an integer from 0 to 3.

### (Item 31)

The compound, or an amino-protected compound when the amino is present on the ring in the 7-side chain, or a pharmaceutically acceptable salt thereof according to any one of Items 1 to 30, wherein R¹⁰ is

1) substituted phenyl or substituted 6-membered heterocyclyl containing 1 to 3 nitrogen atom(s),

2) substituted 9-membered bicyclic aromatic heterocyclyl, or

3) the group represented by the following formula

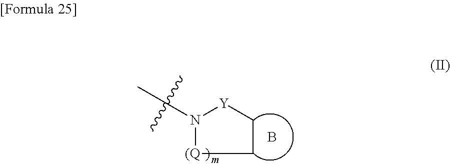

wherein
ring B is substituted carbocycle or substituted heterocycle, the other symbols are defined as above, and the substituents are at least two hydroxyl groups, and the each hydroxyl groups binds to the each adjacent carbon atom which is ring member atom.

### (Item 32)

A pharmaceutical composition, which comprises a compound or an amino-protected compound when the amino is present on the ring in the 7-side chain, or a pharmaceutically acceptable salt thereof according to any one of Items 1 to 31.

### (Item 33)

A pharmaceutical composition, which comprises a compound or an amino-protected compound when the amino is present on the ring in the 7-side chain, or a pharmaceutically acceptable salt thereof according to any one of Items 1 to 31, which possesses antimicrobial activity.

### (Item 34)

The pharmaceutical composition according to Item 32, which is for treating an infectious disease.

### (Item 35)

The compound, or an amino-protected compound when the amino is present on the ring in the 7-side chain, or a pharmaceutically acceptable salt thereof according to any one of Items 1 to 31, which is for treating and/or preventing an infectious disease.

### (Item 36)

A method for treating and/or preventing an infectious disease, characterizes in the step of administering the compound, or an amino-protected compound when the amino is present on the ring in the 7-side chain, or a pharmaceutically acceptable salt thereof according to any one of Items 1 to 31.

### Effects of the Invention

The compounds of the subject invention are useful as a pharmaceutical product in that the compounds have at least one of the following characters:

A) The compounds exhibit potent antimicrobial spectrum against a variety of bacteria including Gram negative bacteria and/or Gram positive bacteria;

B) the compounds exhibit potent antimicrobial activity against beta-lactamase producing Gram negative bacteria;

C) the compounds exhibit potent antimicrobial activity against multidrug-resistant bacteria, in particular, Class B type metallo-beta-lactamase producing Gram negative bacteria;

D) the compounds exhibit potent antimicrobial activity against extended-spectrum beta-lactamase (ESBL) producing bacteria;

E) the compounds do not exhibit cross resistance with known cephem drugs and/or Carbapenem drugs;

F) the compounds do not exhibit side effects such as fever after administration into the body;

G) the compounds have high stability and/or solubility against water; and

H) the compounds have superior characters in pharmacokinetics such as high blood concentration, high absorption of oral preparations, long-acting character or high tissue migration.

### EMBODIMENTS FOR CARRYING OUT THE INVENTION

Hereafter, the subject invention is described with showing embodiments. It should be understood that, throughout the present specification, the expression of a singular form (for example, in the English language, “a”, “an”, “the”, and the like; and in other languages, corresponding articles, adjectives, and the like) includes the concept its plural form unless specified otherwise. Furthermore, it should be understood that the terms used herein are used in a meaning normally used in the art unless specified otherwise. Thus, unless defined otherwise, all technical and scientific terms used herein have the same meaning as those generally understood by those skilled in the art in the field to which the subject invention pertains. If there is a contradiction, the present specification (including definitions) precedes. Each specific definition of terms specifically used herein is described below.

Each term in the present specification is used alone or in combination with another word, and defined as below.

“Halogen” includes fluorine, chlorine, bromine and iodine. Preferably, halogen is fluorine, chlorine or bromine, and more preferably is chlorine.

“Lower alkyl” includes linear or branched alkyl having 1-8 carbons, preferably 1-6 carbons, and more preferably 1-4 carbons, and includes, for example, methyl, ethyl, n-propyl, isopropyl, n-butyl, isobutyl, sec-butyl, tert-butyl, n-pentyl, isopenty, neopentyl, hexyl, isohexyl, n-heptyl, isoheptyl, n-octyl and the like.

“Lower alkylene” includes linear alkylene having 1-8 carbons, preferably 1-6 carbons, more preferably 1-4 carbons, and most preferably one or two carbons, and includes, for example methylene, ethylene, n-propylene, n-butylene, n-pentylyene, n-hexylene, and the like.

“Lower alkenylene” includes linear alkenylene having 2-8 carbons, preferably 2-6 carbons, more preferably 2-4 carbons, and at least one double bond at any position, and includes, for example, vinylene, allylene, propenylene, butenylene, prenylene, butadienylene, pentenylene, pentadienylene, hexenylene, hexadienylene and the like.

“Lower alkynylene” includes linear alkynylene having 2-8 carbons, preferably 2-6 carbons, more preferably 2-4 carbons, and at least one triple bond at any position, and includes, for example, ethynylene, propynylene, buthynylene, pentynylene, hexynylene, and the like.

“Halo lower alkyl” refers to a group in which at least one position of the said “lower alkyl” is substituted with the above “halogen”, and includes, for example, monofluoromethyl, difluoromethyl, trifluoromethyl, monochloromethyl, dichloromethyl, trichloromethyl, monobromomethyl, monofluoroethyl, monochloroethyl, chlorodifluoromethyl, and the like. Preferably, halo lower alkyl is trifluoromethyl, or trichloromethyl.

Substituents of “substituted or unsubstituted amino” or “substituted or unsubstituted carbamoyl” include substituted or unsubstituted lower alkyl (e.g., methyl, ethyl, isopropyl, benzyl, carbamoylalkyl (e.g., carbamoylmethyl), mono- or di-loweralkyl carbamoyl lower alkyl (e.g.: dimethylcarbamoylethyl), hydroxy lower alkyl, heterocycle lower alkyl (e.g., morpholino ethyl, tetrahydro pyranylethyl), alkyloxycarbonyl lower alkyl (e.g., ethoxycarbonylmethyl, ethoxycarbonylethyl), mono- or di-lower alkylamino lower alkyl (e.g., dimethylaminoethyl); lower alkyloxy lower alkyl (e.g., methoxyethyl, ethoxymethyl, ethoxyethyl, isopropoxyethyl, and the like)); acyl (e.g., formyl, substituted or unsubstituted lower alkylcarbonyl (e.g., acetyl, propionyl, butylyl, isobutylyl, valeryl, isovaleryl, pivaloyl, hexanoyl, octanoyl, methoxyethylcarbonyl, 2,2,2-trifluoroethylcarbonyl, alkyloxycarbonylacetyl (e.g., ethoxycarbonylmethylcarbonyl), lower alkyloxy lower alkylcarbonyl (e.g., methoxyethylcarbonyl), lower alkylcarbamoyl lower alkylcarbonyl (e.g., methylcarbamoylethylcarbonyl), substituted or unsubstituted arylcarbonyl (e.g., benzoyl, toluoyl)); substituted or unsubstituted arylalkyl (e.g., benzyl, 4-fluorobenzyl); hydroxy; substituted or unsubstituted lower alkylsulfonyl (e.g., methanesulfonyl, ethanesulfonyl, isopropylsulfonyl, 2,2,2-trifluoroethanesulfonyl, benzyl sulfonyl, methoxyethylsulfonyl); arylsulfonyl optionally having lower alkyl or halogen as a substituent (e.g., benzylsulfonyl, methoxtethylsulfonyl); arylsulfonyl optionally having lower alkyl or halogen as a substituent (e.g., benzenesulfonyl, toluenesulfonyl, 4-fluorobenzenesulfonyl), cycloalkyl (e.g., cyclopropyl); aryl optionally having lower alkyl as a substituent (e.g., phenyl, tolyl); lower alkylaminosulfonyl (e.g., methylaminosulfonyl, dimethylaminosulfonyl); lower alkylaminocarbonyl (e.g., dimethylaminocarbonyl); lower alkyloxycarbonyl (e.g., ethoxycarbonyl); cycloalkylcarbonyl (e.g., cyclopropylcarbonyl, cyclohexylcarbonyl); substituted or unsubstituted sulfamoyl (e.g., sulfamoyl, methylsulfamoyl, dimethylsulfamoyl); lower alkylcarbonylamino (e.g., methylcarbonylamino); heterocycle (e.g., morpholino, tetrahydropyranyl); substituted or unsubstituted amino (e.g., mono- or di-alkyl amino (e.g., dimethylamino), formylamino), and the like.

The above “substituted amino group” or “substituted carbamoyl group” may be mono-substituted or di-substituted with these substituent groups.

“Lower alkenyl” refers to a linear or branched alkenyl having 2 to 8 carbons and having, one or more double bonds on the said “lower alkyl”. Examples thereof include vinyl, 1-propenyl, 2-propenyl, 1-butenyl, 2-butenyl, 3-butenyl, 1,3-butadienyl, 3-methyl-2-butenyl, and the like. Preferred is alkenyl having 2 to 6 carbons, more preferably 2 to 4 carbons.

With regard to an amino group of “substituted or unsubstituted amino” or “substituted or unsubstituted carbamoyl”, two substituents of the amino group may be taken together with the adjacent nitrogen atom to form a nitrogen-containing heterocycle which optionally includes a sulfur atom and/or an oxygen atom in the ring (preferably, the heterocycle is a 5- to 7-membered ring and is preferably saturated). The heterocycle is optionally substituted with oxo or hydroxyl. When a sulfur atom forms the heterocycle, the said sulfur atom is optionally substituted with oxo. Preferred examples thereof include 5- or 6-membered rings such as piperazinyl, piperadino, morpholino, pyrrolodino, 2-oxopiperidino, 2-oxopirrolidino, 4-hydroxymorpholino, and the like.

Substituents of “substituted or unsubstituted lower alkyl” include at least one group selected from Substituent Group Alpha. When substitution is carried out with a plurality of Substituent Group Alpha, the plurality of Substituent Group Alpha may be same or different.

Substituents of “substituted or unsubstituted lower alkylene”, “substituted or unsubstituted lower alkenylene” and “substituted or unsubstituted lower alkynylene” include at least one group selected from Substituent Group Alpha. When substitution is carried out with a plurality of substituents, the substituents may be the same or different.

Substituents of “substituted or unsubstituted lower alkyloxycarbonyl” include at least one group selected from Substituent Group Alpha.

Substituents of “a substituted carbonyloxy group” meaning “—O—C(═O)-substituent” include substituted or unsubstituted lower alkyl, substituted or unsubstituted lower alkenyl, substituted or unsubstituted lower alkynyl, substituted or unsubstituted carbocyclyl, substituted or unsubstituted heterocyclyl, an amino having a heterocyclyl as a substituent, and at least one group selected from Substituent Group Alpha.

“A substituted or unsubstituted acyl group” includes at least one group selected from substituted or unsubstituted lower alkyl, substituted or unsubstituted lower alkenyl, substituted or unsubstituted lower alkynyl, substituted or unsubstituted carbocyclyl, and substituted or unsubstituted heterocyclyl.

“A substituted or unsubstituted acyl group” means a carbonyl group substituted with substituted or unsubstituted lower alkyl, substituted or unsubstituted lower alkenyl, substituted or unsubstituted lower alkynyl, substituted or unsubstituted carbocyclyl or substituted or unsubstituted heterocyclyl.

Substituents of “substituted or unsubstituted, saturated or unsaturated, monocyclic or fused cyclic divalent group containing a quaternary ammonium ion” include substituted or unsubstituted lower alkyl, lower alkylene, at least one group selected from Substituent Group Alpha, or two or more substitutents that are taken together to form a carocyclic group or heterocyclic group. When a substituent is lower alkylene, the said lower alkylene forms a bridged structure by bonding with arbitrary two ring member atoms. The said lower alkylene preferably is a bridged structure formed by bonding with a cationic nitrogen atom and an arbitrary ring member atom.

Here, “Substituent Group Alpha” is a group consisting of halogen, hydroxy, lower alkyl oxy, lower alkylene, hydroxyl, lower alkyloxy, lower alkyloxy lower alkyloxy, carboxy, amino, acylamino, lower alkylamino, imino, hydroxyimino, lower alkyloxyimino, lower alkylthio, carbamoyl, lower alkylcarbamoyl, hydroxy lower alkylcarbamoyl, sulfamoyl, lower alkylsulfamoyl, lower alkylsulfinyl, cyano, nitro, carbocyclyl and heterocyclyl.

The lower alkyl moiety in “lower alkyloxy”, “hydroxy lower alkyloxy”, lower alkyloxy lower alkyloxy”, “lower alkylamino”, “lower alkyloxyimino”, “lower alkylthio”, “lower alkylcarbamoyl”, “hydroxyl lower alkylcarbamoyl”, “lower alkylsulfamoyl”, “lower alkylsulfinyl”, “lower alkylsulfinyl”, “lower alkyloxycarbonyl”, “lower alkylsulfonyl” is defined the same as the above “lower alkyl”.

The lower alkenyl moiety in “lower alkenyloxy” is defined the same as the above “lower alkenyl”.

The aryl moiety in “aryloxy” is defined the same as the above “aryl”.

Preferred embodiments of substituents in “substituted or unsubstituted lower alkyl” include a fluorine atom, a chlorine atom, a bromine atom, hydroxy, carboxy, methoxy, ethoxy, hydroxymethoxy, hydroxyethoxy, methoxymethoxy, methoxyethoxy, amino, acetylamino, methylamino, dimethylamino, imino, hydroxyimino, methoxyimino, methylthio, carbamoyl, methylcarbamoyl, hydroxymethylcarbamoyl, sulfamoyl, methylsulfamoyl, lower alkylsulfamoyl, cyano, nitro, phenyl, cyclopropyl, cyclobutyl, cyclohexyl, pyridyl, morpholinyl, and the like.

Preferred embodiments of “substituted or unsubstituted lower alkyl” include methyl, ethyl, isopropyl, tert-butyl, monofluoromethyl, difluoromethyl, trifluoromethyl, monochloromethyl, dichloromethyl, trichloromethyl, carboxymethyl, carboxyethyl, carbamoylmethyl, carbamoylethyl, hydroxymethyl, hydroxyethyl, methoxymethyl, ethoxymethyl, methoxyethyl, ethoxyethyl, methylthiomethyl, ethylthiomethyl, benzyl, phenethyl, 4-hydroxybenzyl, 4-methoxybenzyl, 4-carboxybenzyl, and the like.

“Carbocyclyl” includes cycloalkyl, cycloalkenyl, aryl and non-aromatic fused carbocyclyl, and the like.

“Cycloalkyl” is a carbocyclyl having 3-10 carbons, preferably 3-8 carbons, more preferably 3-7 carbons, and includes, for example, cyclopropyl, cyclobutyl, cyclopentyl, cyclohexyl, cycloheptyl, cyclooctyl, cyclononyl cyclodecyl, and the like.

“Cycloalkenyl” includes those in which the ring of the cycloalkyl has at least one double bond any position(s), and specifically includes, for example, cyclopropenyl, cyclobutenyl, cyclopentenyl, cyclohexenyl, cycloheptynyl, cyclooctynyl, and cyclohexadienyl, and the like.

“Aryl” includes phenyl, naphthyl, anthryl, phenanthryl, and the like, and in particular, phenyl is preferable.

“Aromatic carbocycle” means a ring derived from aryl as described above.

“Non-aromatic carbocyclyl” includes those selected from the above “cycloalkyl” and “cycloalkenyl” and specifically includes cyclopropyl, cyclobutyl, cyclopentyl, cyclohexyl, cycloheptyl, cyclooctyl, cyclononyl, cyclodecyl, cyclopropenyl, cyclobutenyl, cyclopentenyl, cyclohexenyl, cycloheptynyl, cyclooctynyl, and cyclohexadienyl, and the like.

“Non-aromatic fused carbocyclyl” includes a group in which one or more cyclic group selected from the said “cycloalkyl”, “cycloalkenyl” and “aryl”, and specifically includes, for example, ihdanyl, indenyl, tetrahydronaphthyl and fluorenyl, and the like.

“Carbocycle” includes the above “aromatic carbocycle” and “non-aromatic carbocycle” or “non-aromatic fused carbocycle”.

“Heterocyclyl” includes heterocyclyl having at least one hetero atom arbitrarily selected from O, S, and N in the ring thereof, and specifically includes, for example, 5- or 6-membered heteroaryl such as pyrrolyl, imidazolyl, pyrazolyl, pyridyl, pyridazinyl, pyrimidinyl, pyrazinyl, triazolyl, triazinyl, tetrazolyl, isoxazolyl, oxazolyl, oxadiazolyl, isothiazolyl, thiazolyl, thiadiazolyl, furyl, thienyl, and the like; bicyclic fused heterocyclyl such as indolyl, isoindolyl, indazolyl, indolizinyl, indolinyl, isoindolinyl, quinolyl, isoquinolyl, cinnolinyl, phthalazinyl, quinazolinyl, naphthyridinyl, quinoxalinyl, purinyl, puteridinyl, benzopyranyl, benzimidazolyl, benzotriazolyl, benzisoxazolyl, benzoxazolyl, benzoxadiazolyl, benzisothiazolyl, benzothiazolyl, benzothiadiazolyl, benzofuryl, isobenzofuryl, benzothienyl, benzotriazolyl, imidazopyridyl, pyrazolopyridine, triazolopyridyl, triazolopyridyl, imidazothiazolyl, pyrazinopyridazinyl, quinazolinyl, quinolyl, isoquinolyl, naphthyridinyl, dihydrobenzofuryl, tetrahydroquinolyl, tetrahydroisoquinolyl, dihydrobenzoxazine, tetrahydrobenzothienyl, and the like; tricyclic fused heterocyclyl such as carbazolyl, acridinyl, xanthenyl, phenothiadinyl, phenoxathiinyl, phenoxazinyl, dibenzofuryl, imidazoquinolyl, and the like; non-aromatic heterocyclyl such as dioxanyl, thiiranyl, oxiranyl, ozathiolanyl, azetidinyl, thianyl, thiazolidine, pyrrolidinyl, pyrrolinyl, imidazolidinyl, imidazolinyl, pyrazolidinyl, pyrazolinyl, piperidyl, piperazinyl, morpholinyl, thiomorpholinyl, thiomorpholino, dihydropyridyl, dihydrobenzimidazolyl, tetrahydropyridyl, tetrahydrofuryl, tetrahydropyranyl, tetrahydrothiazolyl, tetrahydroisothiazolyl, dihydrooxazinyl, hexahydroazepinyl, tetrahydrodiazepinyl, and the like. Preferably, heterocyclyl is a 5- or 6-membered heteroaryl or non-aromatic heterocyclyl, and more preferably, a 5- or 6-membered heteroaryl.

“Aromatic heterocycle” means an aromatic ring, which is monocyclic or ciclyclic or more, having same or different one or more hetero atom selected independently from O, S and N. Aromatic heterocyclyl which is two or more cycles also includes the above “aromatic carbocycle” condensed in aromatic heterocyclyl which is one or more cycle(s).

“Non-aromatic heterocyclyl” means a group which does not show aromatic character of the above “heterocyclyl”.

“Heterocycle” means a ring derived from the above “heterocyclyl”.

Substituents of “substituted or unsubstituted carbocyclyl”, “substituted or unsubstituted heterocyclyl”, “substituted or unsubstituted non-aromatic carbocyclyl”, “substituted or unsubstituted non-aromatic heterocyclyl”, “substituted or unsubstituted phenyl”, “substituted or unsubstituted 6-membered heterocyclyl having 1-3 nitrogen atoms”, “substituted or unsubstituted 9-membered bicyclic aromatic heterocycle”, “substituted or unsubstituted carbocycle” and “substituted or unsubstituted heterocycle” include substituted or unsubstituted lower alkyl, and at least one or more group selected from Substituent Group Alpha.

Preferred embodiments of substituents in “substituted or unsubstituted carbocyclyl”, substituted or unsubstituted heterocyclyl”, “substituted or unsubstituted non-aromatic carbocyclyl”, “substituted or unsubstituted non-aromatic heterocyclyl”, “substituted or unsubstituted phenyl”, “substituted or unsubstituted 6-membered heterocyclyl having 1-3 nitrogen atoms”, “substituted or unsubstituted 9-membered bicyclic aromatic heterocycle”, “substituted or unsubstituted carbocycle” and “substituted or unsubstituted heterocycle” methyl, ethyl, isopropyl, tert-butyl, a fluorine atom, a chlorine atom, a bromine atom, hydroxy, carboxy, methoxy, ethoxy, hydroxymethoxy, hydroxyethoxy, methoxymethoxy, methoxyethoxy, amino, acetylamino, methylamino, dimethylamino, imino, hydroxyimino, methoxyimino, methylthio, carbamoyl, methylcarbamoyl, hydroxymethylcarbamoyl, cyano, nitro, phenyl, cyclopropyl, cyclobutyl, cyclohexyl, pyridyl, morpholinyl, and the like.

Preferred embodiments of substituents in “substituted or unsubstituted phenyl”, “substituted or unsubstituted 6-membered heterocyclyl having 1-3 nitrogen atoms”, “substituted or unsubstituted 9-membered bicyclic aromatic heterocycle”, “substituted or unsubstituted carbocycle” and “substituted or unsubstituted heterocycle” are two adjacent hydroxy groups, the said rings are optionally substituted more a fluorine atom, a chlorine atom, a bromine atom, hydroxy, methyl, methoxy and/or carboxy.

“6-membered heterocyclyl having 1-3 nitrogen atoms” includes pyridyl, pyrimidinyl, pyridazinyl, pyrazinyl, 1,3,5-triazinyl, 1,2,4-triazinyl, 1,2,3-triazinyl, and the like.

“9-membered bicyclic aromatic heterocyclyl” includes indolyl, indazolyl, indorizinyl, benzimidazolyl, benzisoxazolyl, benzoxazolyl, benzoxadiazolyl, benzoisothiazolyl, benzothiazolyl, benzothiadiazolyl, benzofuryl, isobenzofuryl, benzothienyl, imidazopyridyl, pyrazolopyridyl, triazolopyridyl, and the like.

“5- or 6-membered heterocyclyl having 1-3 nitrogen atoms” includes pyrrolyl, imidazolyl, pyrazolyl, pyridyl, pyridazinyl, pyrimidinyl, pyrazinyl, triazolyl, triadinyl, isoxazolyl, oxazolyl, oxadiazolyl, isothiazolyl, thiazolyl, thiadiazolyl, furyl, thienyl, and the like.

Examples or embodiments of each site of Formula (I) are provided below. However, the scope of the subject invention is not limited to those described below.

“W” is —CH₂—, —S— or —O—. Preferably “W” is —CH₂—.

“U” is —CH₂—, —S—, —S(═O)— or —O—, when “W” is —CH₂—. Preferably, “U” is —S— or —S(═O)—, more preferably “U” is —S—.

“U” is —CH₂—, when W is —S— or —O—.

“L” is substituted or unsubstituted lower alkylene or substituted or unsubstituted lower alkenylene. Preferably, “L” is —CH₂—, —CH═CH—, —CH₂—CH═CH— or —CH═CH—CH₂—, more preferably “L” is —CH₂—. Herein, binging pattern of double bond between carbon atoms in “L” may be cis-configuration, trans-configuration, or mixture thereof.

Embodiments of the ring of “substituted or unsubstituted carbocyclyl or substituted or unsubstituted heterocyclyl” include 5- or 6-membered ring. Preferred examples include phenyl, hydroxyphenyl, phenyl having halogen as substituent(s), aminothiazole, aminothiazole which has halogen(s) as substituent(s), aminothiadiazole, thiophen, furan, benzothiazole, pyridine, pyrimidine, pyridazine, amino pyridine, and the like.

More preferred examples of heterocyclyl include the group as follows:

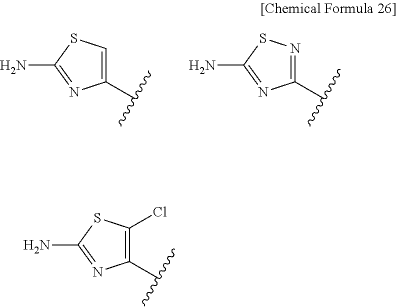 [SMILES: *c1csc(N)n1 ; *c1nsc(N)n1 ; *c1nc(N)sc1Cl]

When R^{2B} is a hydrogen atom, examples of R^{2A} include a hydrogen atom, substituted or unsubstituted amino, —SO₃H, substituted or unsubstituted aminosulfonyl, carboxy, substituted or unsubstituted lower alkyloxycarbonyl, substituted or unsubstituted carbamoyl, hydroxy, or substituted carbonyloxy, and the like. For example, preferred examples of the group represented by Formula:

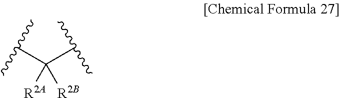

include the substituted amino shown below:

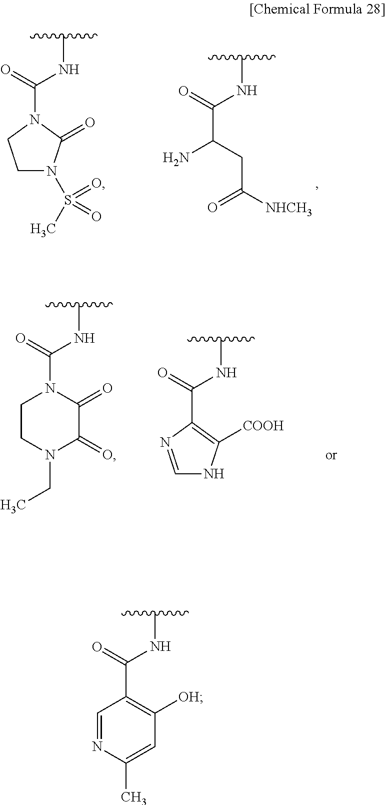 [SMILES: *NC(=O)N1CCN(S(C)(=O)=O)C1=O ; *NC(=O)C(N)CC(=O)NC ; *NC(=O)N1CCN(CC)C(=O)C1=O ; *NC(=O)c1nc[nH]c1C(=O)O ; *NC(=O)c1cnc(C)cc1O]

the substituted aminosulfonyl shown below:

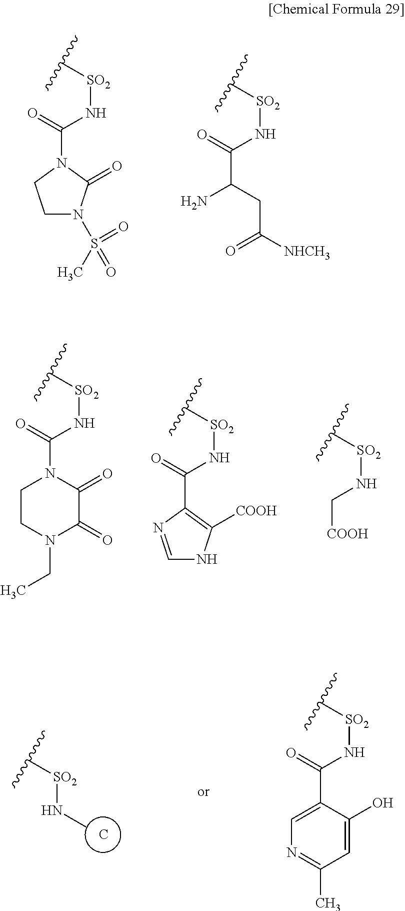

wherein ring C represents substituted or unsubstituted heterocyclyl; the substituted carbamoyl shown below:

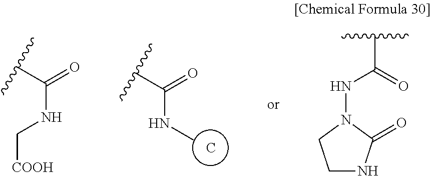

wherein ring C represents substituted or unsubstituted heterocyclyl; or the substituted carbonyloxy shown below:

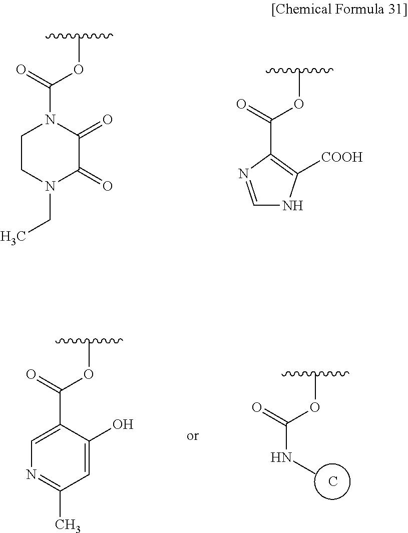

wherein ring C represents substituted or unsubstituted heterocyclyl.

Alternatively, R^{2A} and R^{2B} may be taken together to form substituted or unsubstituted methylidene. A preferable example is a group represented by Formula shown below:

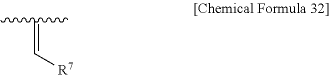

wherein R⁷ is substituted or unsubstituted lower alkyl. Herein, binging pattern of double bond between carbon atoms may be cis-configuration, trans-configuration, or mixture thereof.

The following group is preferable.

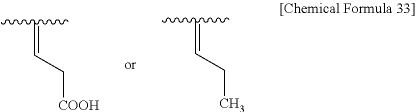

Also, R^{2A} and R^{2B} may be taken together to form substituted or unsubstituted hydroxyimino. A preferable example is the group shown below:

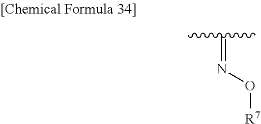

wherein R⁷ is as defined above.

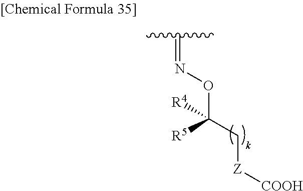

wherein each symbol is as defined above is preferred.

Examples of “R⁴ and R⁵” include a hydrogen atom, a fluorine atom, a chlorine atom, hydroxy, carboxy, methyl, ethyl, isopropyl, tert-butyl, monofluoromethyl, difluoromethyl, trifluoromethyl, carboxymethyl, hydroxyethyl, carbamoylmethyl, carbamoylethyl, hydroxymethyl, hydroxyethyl, methoxymethyl, ethoxymethyl, methoxyethyl, ethoxyethyl, methylthiomethyl, ethylthiomethyl, benzyl, 4-hydroxybenzyl, 4-methoxybenzyl, 4-carboxy benzyl, 3,4-dihydroxyphenyl, naphthyl, cyclopropyl, cyclobutyl, cyclopentyl, cyclohexyl, pyrrolyl, imidazolyl, pyrazolyl, pyridyl, pyridazinyl, pyrimidinyl, pyrazinyl, triazolyl, triazinyl, tetrazolyl, isoxazolyl, oxazolyl, oxadiazolyl, isothiazolyl, thiazolyl, thiadiazolyl, furyl, and thienyl, and the like.

Preferred combinations of R⁴ and R⁵ include, as (R⁴, R⁵), (a hydrogen atom, a hydrogen atom), (methyl, a hydrogen atom), (a hydrogen atom, methyl), (methyl, methyl), (ethyl, a hydrogen atom), (hydrogen atom, ethyl), (ethyl, ethyl), (phenyl, a hydrogen atom),

(a hydrogen atom, phenyl), (dihydroxyphenyl, a hydrogen atom), (a hydrogen atom, dihydroxyphenyl), (carboxymethyl, a hydrogen atom), (a hydrogen atom, carboxymethy), (carboxyethyl, a hydrogen atom), (a hydrogen atom, carboxyethyl), (hydroxyethyl, a hydrogen atom), (a hydrogen atom, hydroxyethyl), (carbamoymethyl, a hydrogen atom), (a hydrogen atom, carbamoylmethyl), (trifluoromethyl, a hydrogen atom), (carboxy, a hydrogen atom), (carbamoylethyl, a hydrogen atom), (benzyl, hydrogen atom), (dihydroxybenzyl, a hydrogen atom), and the like.

Preferred examples of the above substituted hydroxyimino include the groups shown below:

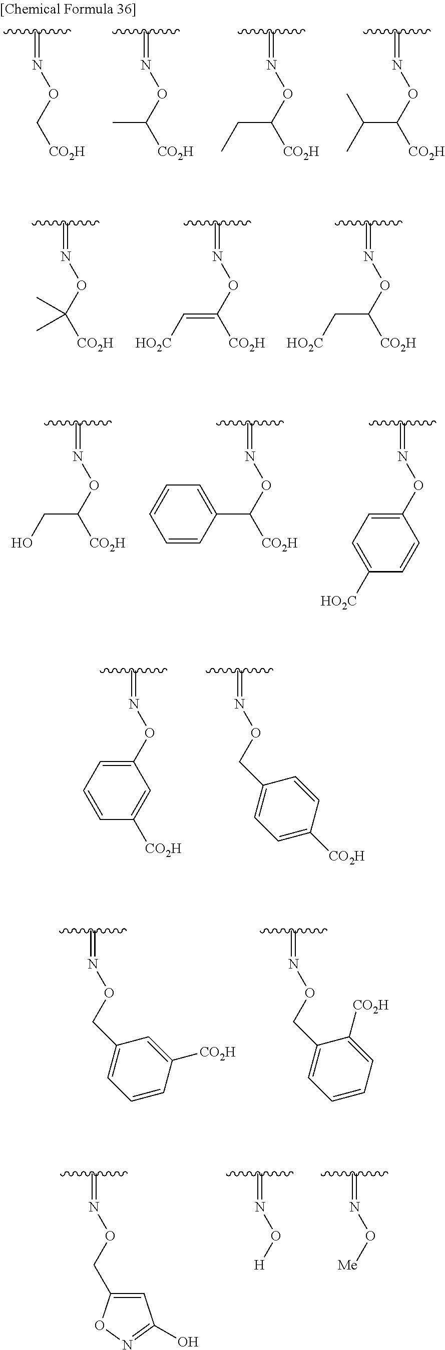

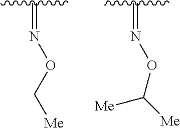 [SMILES: *=NOCC ; *=NOC(C)C]

More preferred examples of the above substituted hydroxyimino include the groups shown below:

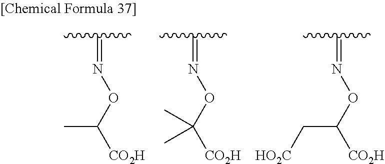

In the case where “R⁴ and R⁵ may be taken together with a neighboring atom to form substituted or unsubstituted carbocycle or substituted or unsubstituted heterocycle”, R⁴ and R⁵ in Formula:

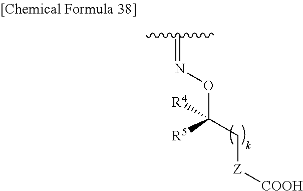

wherein each symbol is as defined above, may form cycloalkane, cycloalkene, or a or non-aromatic heterocycle which optionally substituted on the ring with a group selected from Substituent Group Alpha. For example,

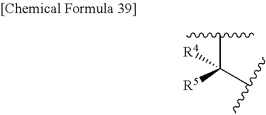

can be a formula shown below:

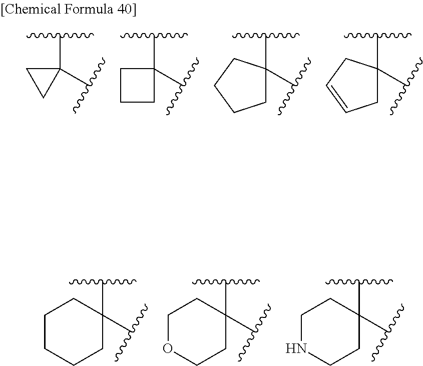

substituted or unsubstituted on the ring with a group selected from Substituent Group Alpha.

Examples of “Z” include a single bond, phenyl, pyridyl, and the like. A single bond is particularly preferable.

“k” is preferably an integer of 0 or 1, and 0 is particularly preferable.

Preferred examples of these embodiments include:

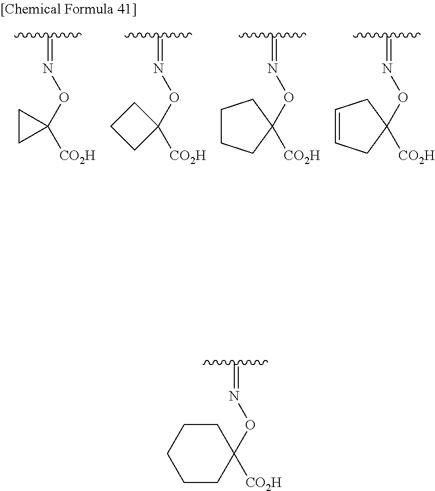 [SMILES: *=NOC1(C(=O)O)CC1 ; *=NOC1(C(=O)O)CCC1 ; *=NOC1(C(=O)O)CCCC1 ; *=NOC1(C(=O)O)CC=CC1 ; *=NOC1(C(=O)O)CCCCC1]

The term “bioisoster” as used herein refers to a group having chemical and physical similarities that provides similar biological properties. Accordingly, “a bioisoster of carboxylate anion (—COO⁻)” of the subject invention refers to any group that provides biological properties similar to those provided by carboxylate anion, specifically refers to a group that is comparatively similar to “carboxylate anion (—COO⁻)” in its chemical structure, that is expected for physical properties, such as acidity, water solubility and/or disposition, equivalent to those of “carboxylate anion (—COO⁻)”, and that has an acidic proton. The said acidic proton moiety may form a salt, such as an alkali metal salt (e.g., sodium salt). Examples can be found in literatures, such as J. Med. Chem. 1992, 35, 1176-1183, J. Med. Chem. 1993, 36, 2485-2493, J. Med. Chem. 1992, 35, 3691-3698, J. Med. Chem. 1995, 38, 617-628, Med. Res. Rev. 1983, 3, 91-118, J. Med. Chem. 2001, 44, 1560-1563, Bioorganic & Medicinal Chemistry Letters, Vol. 4, No. 1, 41-44, 1994, and the like. Preferably, it is selected from —SO₃⁻, —SO₂—N⁻—R¹³, —PO⁻—(OR¹³), —PO₂⁻—(OR¹³), —N⁻—CO—R¹³, —CO—N⁻—OR¹³, —CO—NH—N⁻—SO₂—R¹³, —CO—N⁻—SO₂—R¹³, —CO—CH═C(O⁻)—R¹³, —N⁻—SO₂—R¹³, —CO—N⁻—SO₂—R¹³, —N⁻—SO₂—R¹³, —CO—N⁻—CO—R¹³, —CO—N⁻—SO₂—R¹³, —N⁻—CO—R¹³

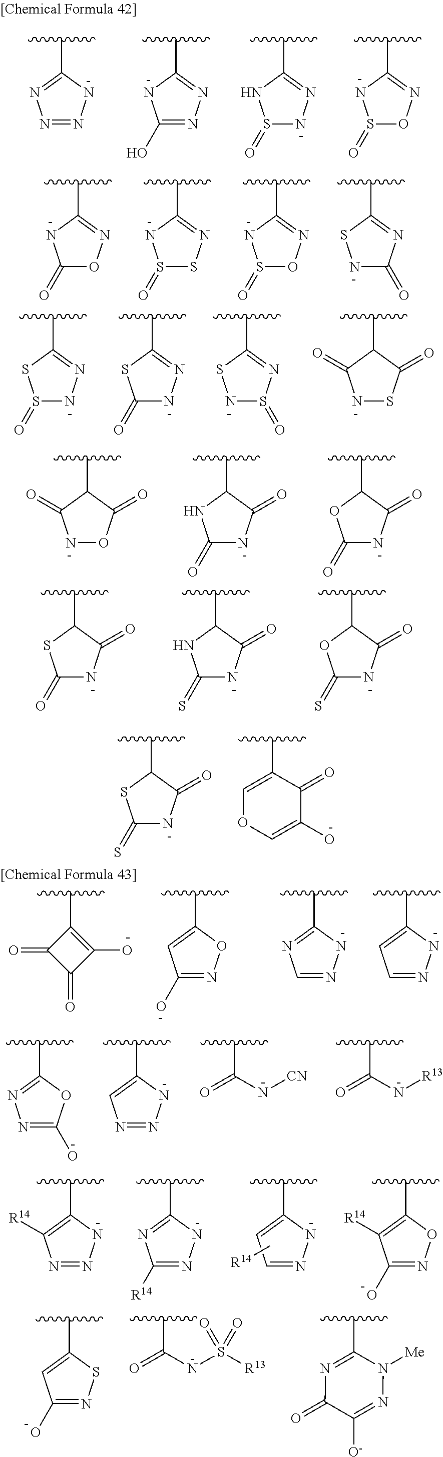

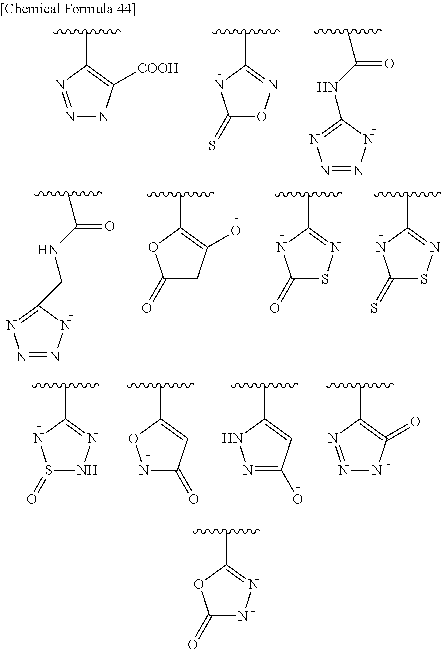

wherein R¹³ is selected from the group consisting of hydrogen, hydroxy, halogen, substituted or unsubstituted lower alkyl, substituted or unsubstituted lower alkenyl, substituted or unsubstituted lower alkynyl, substituted or unsubstituted lower alkyloxy, substituted or unsubstituted amino, lower alkenyloxy, substituted or unsubstituted aryloxy, cyano, nitro, imino, mercapto, lower alkylthio, lower alkylsulfonyl, substituted or unsubstituted carbocyclyl, substituted or unsubstituted heterocyclyl and —CO₂R¹⁷, wherein R¹⁷ is hydrogen, lower alkyl or lower alkenyl; and R¹⁴ is an electron-withdrawing group. The group represented by Formula:

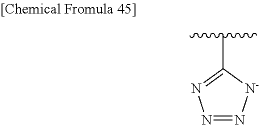 [SMILES: *c1nnn[n-]1]

is more preferable.

R¹⁴ is not limited so long as it is an electron-withdrawing group. Preferred examples of R¹⁴ include fluorine, —CHF₂, —CF₃, —CONH₂, —CN, —C═N—OH, —SO₂CH₃ or —SO₂NH₂, and the like.

“R³” is preferably a hydrogen atom, or —OCH₃, and more preferably a hydrogen atom.

E is a substituted or unsubstituted, cyclic or non-cyclic divalent group having a quaternary ammonium ion. Preferably E is a cyclic group, more preferably is substituted or unsubstituted, saturated or unsaturated, monocyclic or polycyclic divalent group containing a quaternary ammonium ion, which includes the group represented by Formula:

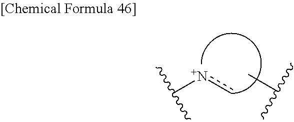

wherein

the dashed line is a bond in the ring;

the bond attached to the cationic nitrogen atom binds to L, and the other bond binds to G;

provided,

when a cationic nitrogen atom binds to G, the dashed line is absent, and

when a cationic nitrogen atom does not bind to G, the dashed line is a single bond between the cationic nitrogen atom and a neighboring atom or an lower alkylene connecting the cationic nitrogen atom and any ring member atom other than the said neighboring atom.

or the group represented by Formula:

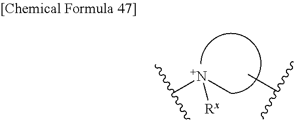

wherein

the bond attached to the cationic nitrogen atom binds to L, and the other bond binds to G;

R^{x} is substituted or unsubstituted lower alkyl.

The monocyclic group of “saturated or unsaturated monocyclic divalent group containing a quaternary ammonium ion” means a saturated or unsaturated monocyclic group consisting of 3 to 8 atoms containing a cationic nitrogen atom, preferably 5 to 7 atoms. Herein, the ring member atoms may include hetero atoms such as oxygen atom, sulfur atom or nitrogen atom, and the like. For example, the monocyclic group includes aziridinium, azetidinium, pyrrolidinium, imidazolium, piperidinium, pyrrolinium, piperadinium, pyridinium, molpholinium, homopiperidinium, homopiperadinium, and the like. Preferable examples include the groups represented by the below formula:

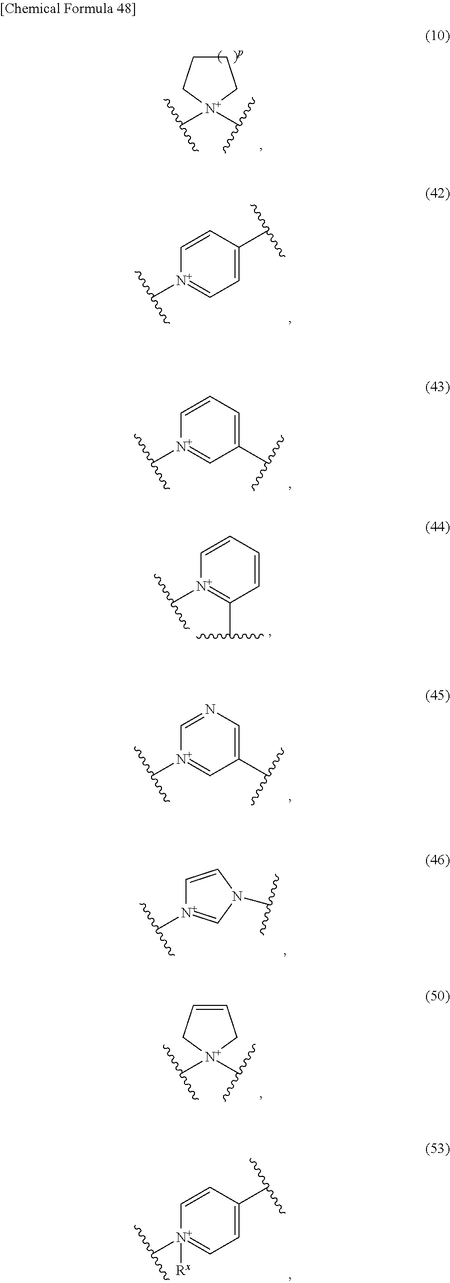

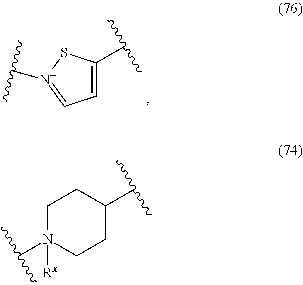

wherein p is an integer from 1 to 3, R^{x} is substituted or unsubstituted lower alkyl.

Additionally, “saturated or unsaturated polycyclic divalent group containing a quaternary ammonium ion” means saturated or unsaturated polycyclic group consisting of from 6 to 15 atoms containing a cationic nitrogen atom. Herein, the ring member atoms may include hetero atoms such as oxygen atoms, sulfur atoms, or nitrogen atoms, and the like. The number of ring atom preferably is 6-10 atoms. Herein, the polycyclic group includes fused ring groups, spiro ring groups, bridged ring groups, and the like. The bridged ring group means the ring system consisting of two more than rings while sharing two or more atoms each other. Preferred examples is saturated fused ring or bridged ring which consist of 6-10 atoms including a cationic nitrogen atom. In particular, the following groups are represented:

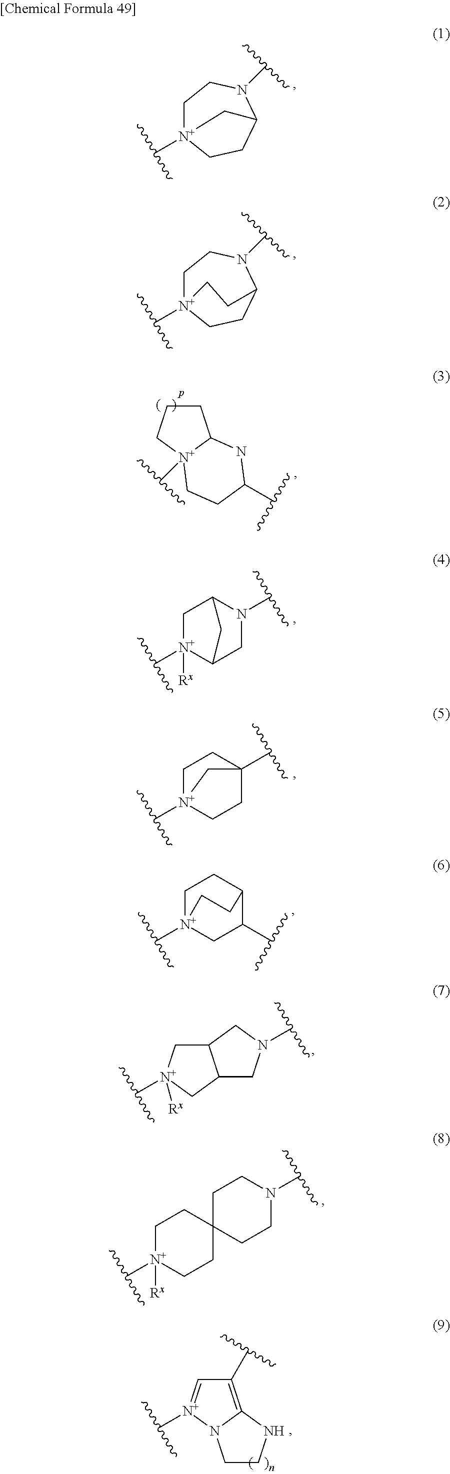

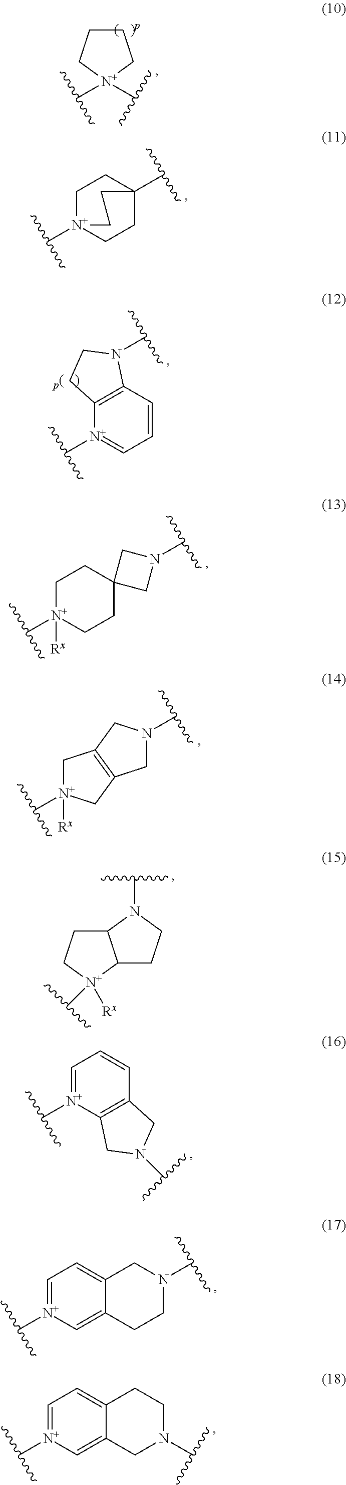

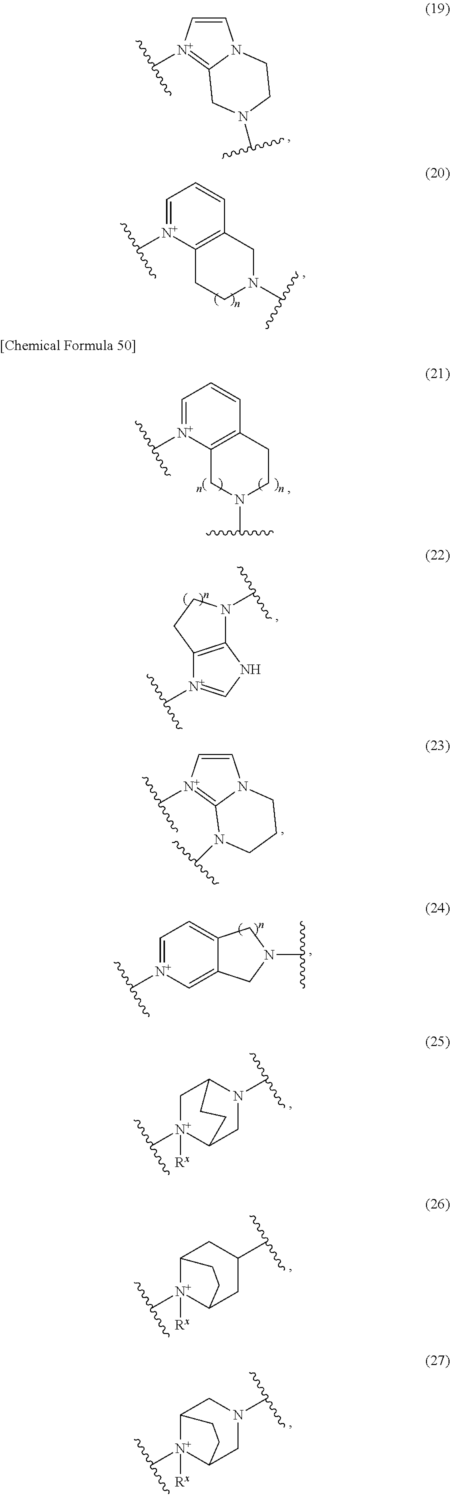

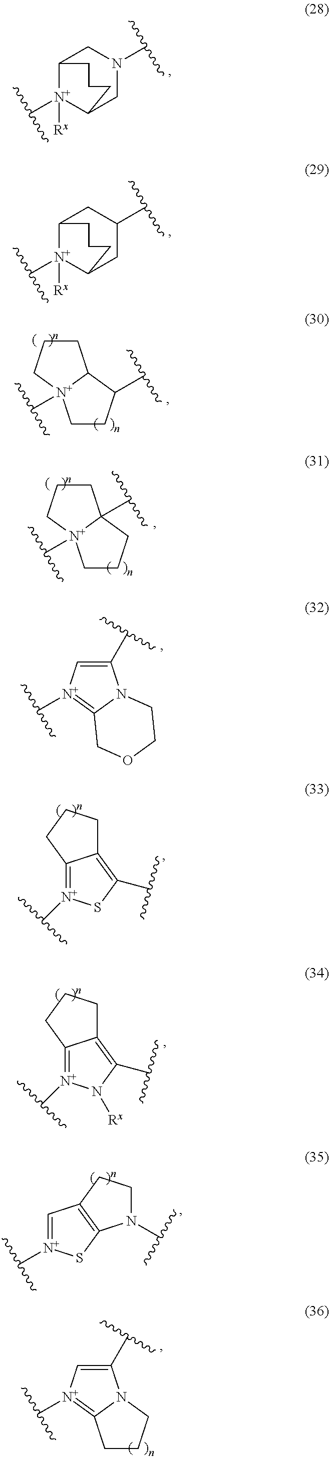

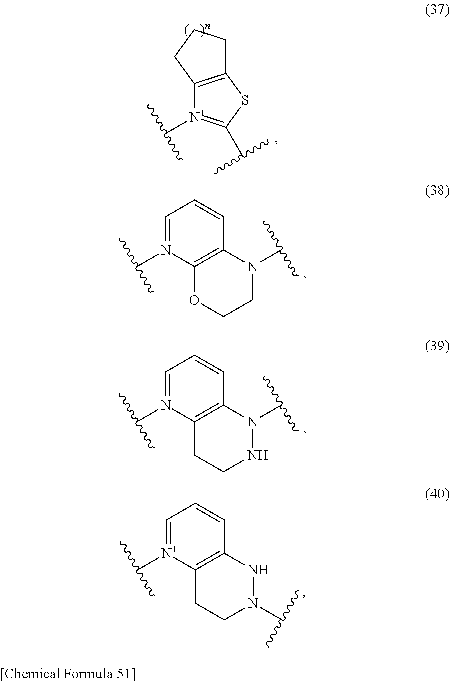

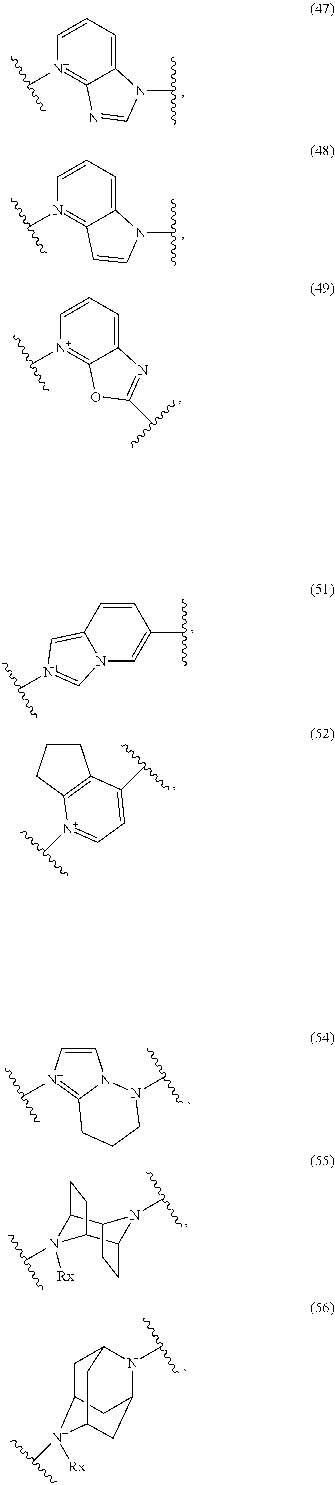

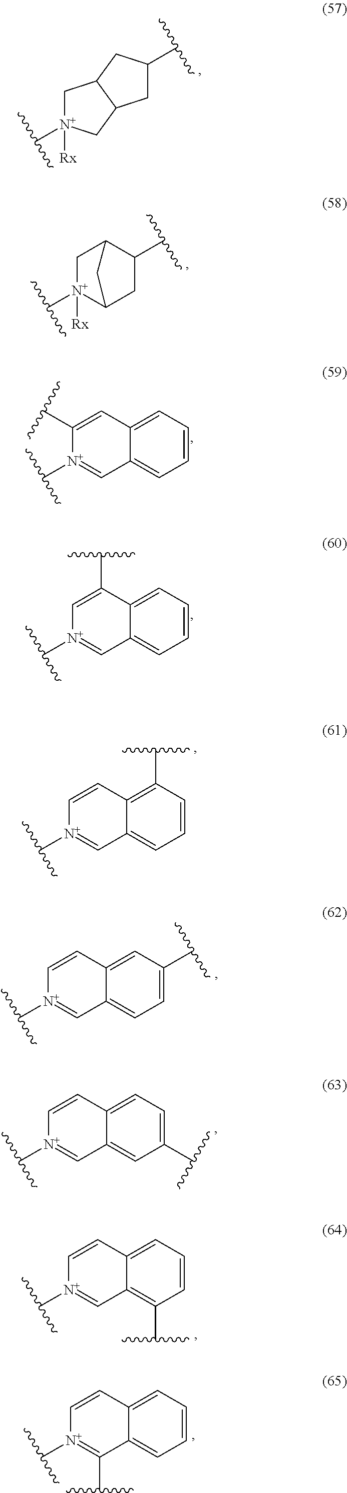

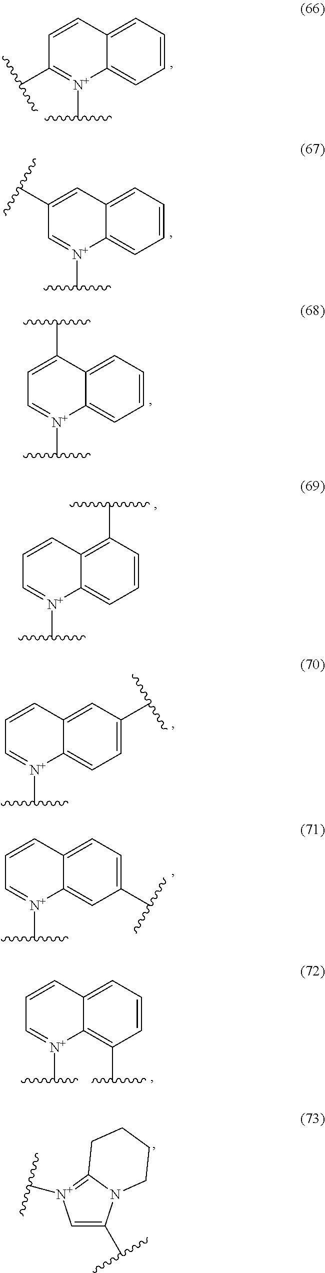

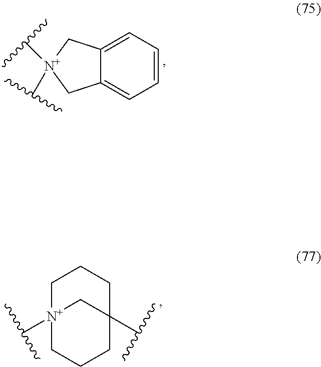 [SMILES: *[N+]1(*)Cc2ccccc2C1 ; *C12CCC[N+](*)(CCC1)C2]

wherein the bond attached to the quaternary nitrogen atom binds to L, and the other bond binds to G; p is an integer from 1 to 3; n is 1 or 2, R^{x} is each independently substituted or unsubstituted lower alkyl.

When E is non-cyclic group, E is preferably the group represented by the following formula:

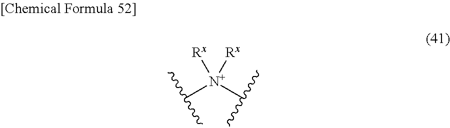

wherein R^{x} is each independently substituted or unsubstituted lower alkyl.

E is preferably selected from the above Formulae (1) to (77) substituted or unsubstituted on the ring. The said substituent is same or different one or more group(s) selected from substituted or unsubstituted lower alkyl and Substituent Group Alpha. Preferred embodiments of such substituent include methyl, ethyl, isopropyl, tert-butyl, a fluorine atom, a chlorine atom, a bromine atom, hydroxy, carboxy, methoxy, ethoxy, hydroxymethoxy, hydroxyethoxy, methoxymethoxy, imino, hydroxyimino, methoxyimino, methylthio, carbamoyl, methylcarbamoyl, hydroxymethylcarbamoyl, sulfamoyl, methylsulfamoyl, lower alkylsulfamoyl, cyano, nitro, phenyl, cyclopropyl, cyclobutyl, cyclohexyl, pyridyl, morpholinyl, and the like. More preferred embodiments include a ring unsubstituted or mono- or di-substituted with a hydroxy. Such ring mono- or di-substituted with a hydroxy may be substituted additionally with another substitutent.

More preferred examples of E is a group selected from the group consisting of the above Formulae (1) to (7), (10) to (12), (14), (25) to (29), (31), (41) to (44), (47), (50), (52), (53), (64) and (73).

Particularly, a group selected from the group consisting of the above Formulae (1), (2), (5), (7), (10), (11), (26) to (29), (31) and (41) is preferable.
Moreover, a group selected from the group consisting of the above Formulae (2), (5), (10), (11), and (26) is more preferable.

G is preferably a single bond, or substituted or unsubstituted lower alkylene. More preferably, G is a single bond methylene or ethylene.

D is preferably a single bond, —C(═O)—, —O—C(═O)—, —C(═O)—O—, —C(═O)—C(═O)—NR⁶—, —NR⁶—C(═O)—C(═O)—, —C(═O)—NR⁶—C(═O)—, —C(═O)—C(═O)—, —O—, —NR⁶—NR⁶—C(═)—, —C(═O)—NR⁶—NR⁶—, —N═N—C(═O)—, —C(═O)—N═N—, —C═N—NR⁶—C(═O)—, —C═N—C(═O)—, —N═CR⁶—C(═O)—, —C═N—C(═O)—NR⁶—, —NR⁶—C(═O)—C(═N—OR⁶)—, —C(═N—OR⁶)—C(═O)—NR⁶—, —NR⁶—C(═N—OR⁶)—, —C(═N—OR⁶)—NR⁶, —C(═O)—C(═N—OR⁶)—, —C(═N—OR⁶)—C(═O)—, —S—, —S(═O)—, —S(═O)₂—NR⁶—, —NR⁶—S(═O)₂—, —NR⁶—CH₂—, —CH₂—NR⁶—, —S(═O)₂—, or —NR⁶—.

More preferably D is a single bond, —NR⁶—C(═O)—C(═O)—, —C(═O)—C(═O)—, —O—, —NR⁶—C(═O)—C(═N—OR⁶)—, —C(═O)—C(═N—OR⁶)— or —NR⁶—. More preferably a single bond, —NR⁶—C(═O)—C(═O)— or —NR⁶—C(═O)—C(═N—OR⁶).

For example, a group represented by —C(═N—OR⁶)— means a formula:

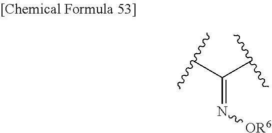

wherein R⁶ is defined as above;
the wavy line means that the bond is cic-configuration, trans-configuration or the mixture thereof; that is, the groups represented by Formula:

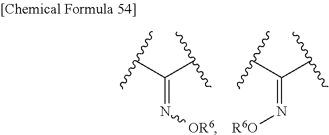

wherein R⁶ is defined as above.
and the mixture thereof is included.

One of the preferred embodiments of G-D-R¹⁰ is a group selected from the following formula:

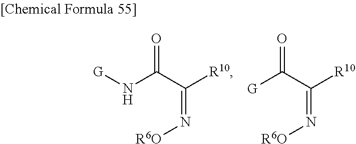

wherein each symbol is defined as above.

R⁶ is preferably a hydrogen atom, unsubstituted lower alkyl, halo lower alkyl, carboxy, carbamoyl or lower alkyl substituted with lower alkyloxycarbonyl.

R⁶ is more preferably a hydrogen atom, unsubstituted lower alkyl, or lower alkyl substituted with carboxy.

Further more preferably, R⁶ is a group selected from the groups consisting of a hydrogen atom, methyl, ethyl and a group represented by Formula:

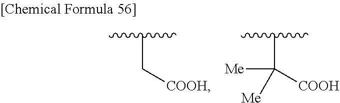 [SMILES: *CC(=O)O ; *C(C)(C)C(=O)O]

wherein “Me” means methyl.

When R¹⁰ is “1) substituted or unsubstituted phenyl or substituted or unsubstituted 6-membered heterocyclyl containing 1 to 3 nitrogen atom(s)”, preferred example is substituted phenyl, and preferred embodiments of the said substituent include hydroxy and/or halogen. More preferred embodiments include phenyl substituted with at least two hydroxyl groups which bind to each of adjacent ring member atoms, the said phenyl may be additionally substituted.

Preferred example of “substituted or unsubstituted 6-membered heterocyclyl containing 1 to 3 nitrogen atom” is substituted pyridyl, and preferred embodiments of the said substituent include hydroxy and/or halogen. More preferred embodiments include pyridyl substituted with at least two adjacent hydroxyl groups, and the said pyridyl may be additionally substituted with halogen.

Preferred example of R¹⁰ is phenyl substituted with at least two adjacent hydroxyl groups or 2-pyridyl substituted with at least two adjacent hydroxyl groups, and additionally these rings are optionally substituted with halogen.

For example, the group represented by the following formula is exemplified:

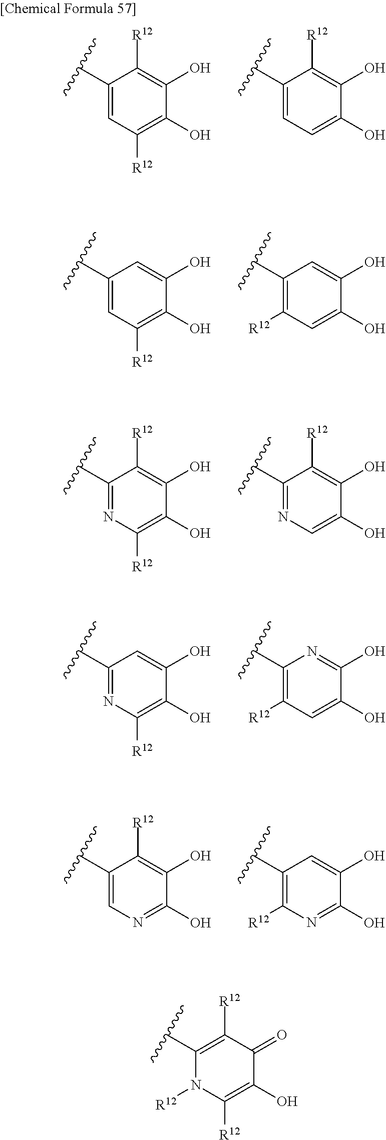

wherein R¹² is each independently a hydrogen atom, halogen, hydroxy, —CN, —C(═O)—R¹⁵, —C(═O)—OH, —C(═O)—OR¹⁵ or OR¹⁵; R¹⁵ is each independently lower alkyl or halo lower alkyl.

More preferred example is exemplified by the following formula:

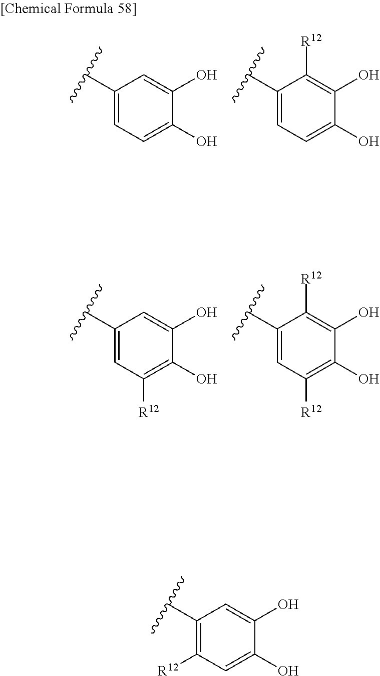

wherein R¹² is each independently a fluorine atom or a chlorine atom.

“Substituted or unsubstituted 9-membered bycyclic aromatic heterocyclyl” means substituted or unsubstituted bycyclic aromatic heterocyclyl consisting of 9 atoms, in particular the group represented by the following Formula is exemplified:

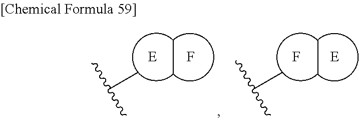

wherein ring E is substituted or unsubstituted 5-membered aromatic heterocycle; ring F is substituted or unsubstituted 6-membered aromatic heterocycle; and the bond attached to ring E or ring F binds to D.

When R¹⁰ is “2) substituted or unsubstituted 9-membered bicyclic aromatic heterocyclyl”, preferred embodiments of the said substituent include hydroxy and/or halogen. More preferred embodiments include bicyclic aromatic heterocyclyl substituted with at least two adjacent hydroxyl groups, and the said ring may be additionally substituted with halogen.

When R¹⁰ is “2) substituted or unsubstituted 9-membered bicyclic aromatic heterocyclyl”, preferred examples include the group represented by Formula:

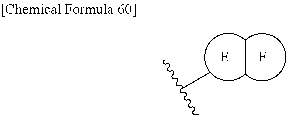

wherein ring E is substituted or unsubstituted 5-membered aromatic heterocycle, ring F is substituted or unsubstituted 6-membered aromatic heterocycle, the bond attached to ring E binds to D. More preferred examples include substituted benzisoxazolyl or substituted benzimidazolyl. Preferred embodiments of the said substituent are hydroxyl and/or halogen. More preferred embodiments include benzisozazolyl or benzimidazolyl which are substituted with at least two adjacent hydroxyl groups, and additionally the groups are optionally substituted with halogen.

For example, the groups are exemplified as follows:

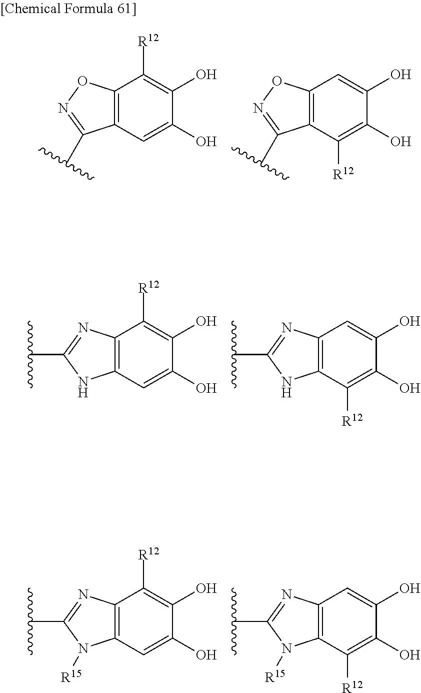

wherein R¹² is independently a hydrogen atom, halogen, hydroxy, —CN, —C(═O)—R¹⁵, —C(═O)—OH, —C(═O)—OR¹⁵ or OR¹⁵, R¹⁵ is each independently lower alkyl or halo lower alkyl.

When R¹⁰ is “3) a group represented by Formula:

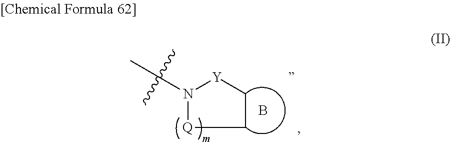

ring B is substituted or unsubstituted carbocycle or substituted or unsubstituted heterocycle. The said ring is preferably 5- or 6-membered ring. Preferred examples of ring B include substituted phenyl, substituted pyridyl, and the like. Preferred embodiments of the said substituent are hydroxy and/or halogen. More preferred embodiments of ring B include phenyl or pyridyl which are substituted with at least two adjacent hydroxy groups, and additionally the rings optionally substituted with one or more halogen(s).

Y is —C(═O)— or —S(═O)₂—, preferably is —C(═O)—.

Q is each independently —O—, —S—, —NR⁸—, —CR⁸R⁹—, —C(═O)—, —S(═O)₂— or —N═CH—, preferably is —O—, —S—, —NR⁸—, —CR⁸R⁹—, —C(═O)— or —N═CH—. More preferably Q is —CR⁸R⁹—, —C(═O)— or —N═CH—.

M is an integer from 1 to 3, preferably is 1 or 2, more preferably is 1.

When R¹⁰ is a group represented by Formula:

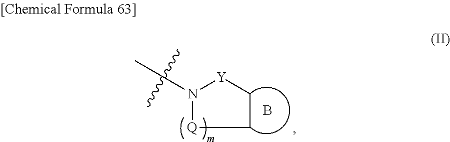

preferred examples include the group represented by Formula:

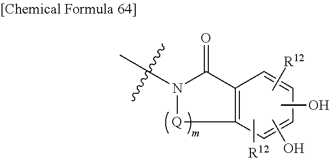

wherein m is 1 or 2,
when m is 1, Q is —O—, —S—, —NR⁸—, —CR⁸R⁹—, —C(═O)— or —N═CH—,
when m is 2, Q is each independently —O—, —S—, —NR⁸— or —C(═O)—,
R¹² is each independently a hydrogen atom, halogen, —CN—, hydroxy or OR¹⁵, and
R¹⁵ is lower alkyl or halo lower alkyl.

For example, the groups represented by the following formula are exemplified:

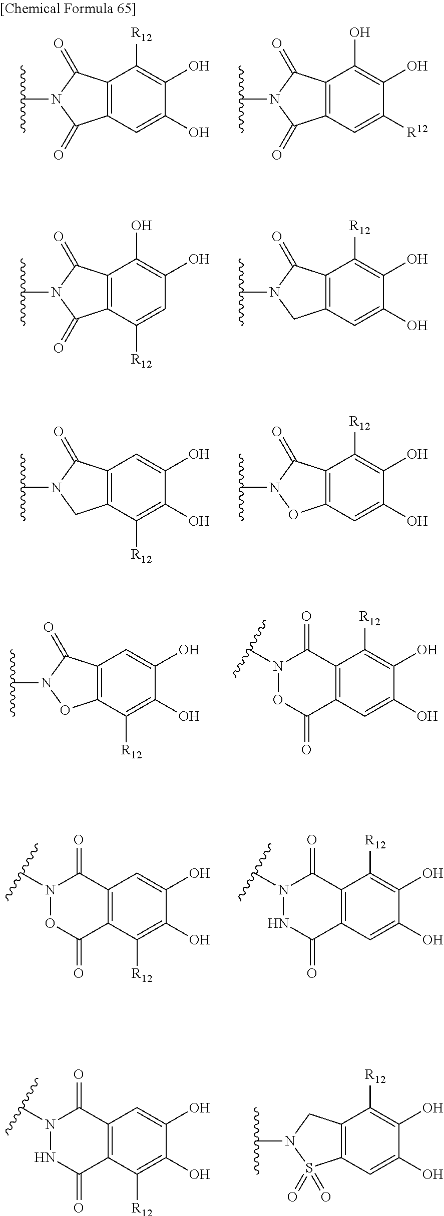

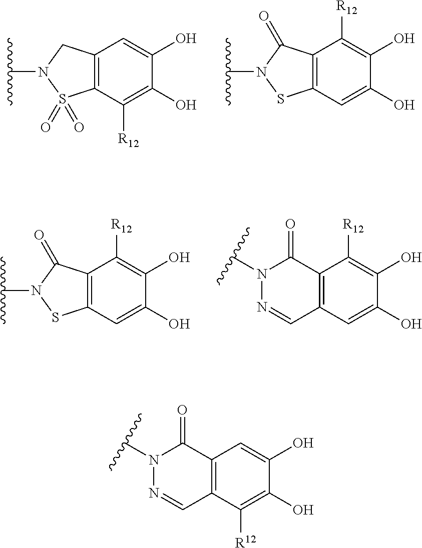

wherein R¹² is each independently a hydrogen atom, halogen, hydroxy, —CN, —C(═O)—R¹⁵, —C(═O)—OH, —C(═O)—OR¹⁵ or OR¹⁵, R¹⁵ is each independently lower alkyl or halo lower alkyl. More preferred embodiments include a group represented as follows:

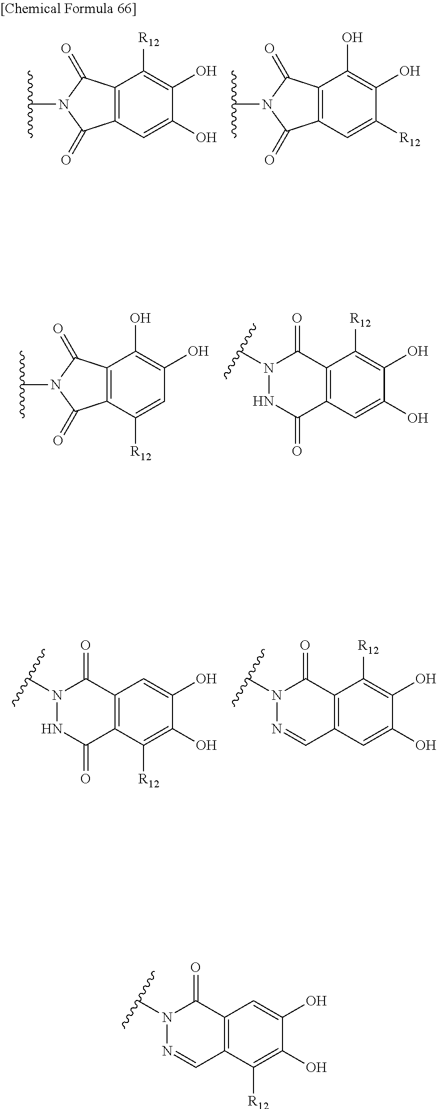

wherein R¹² is defined as above.

More preferred embodiments include a group represented as follows:

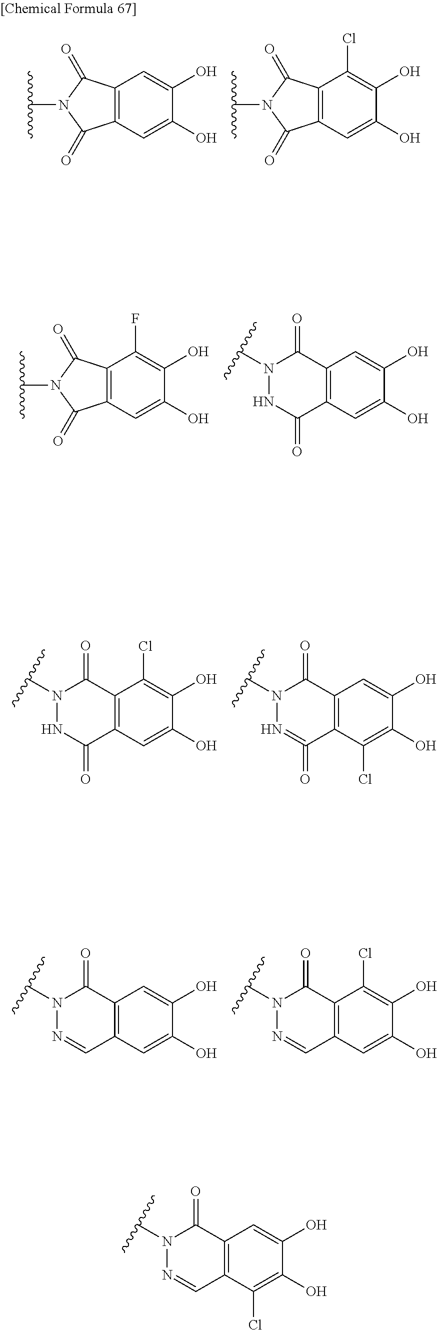

R¹² is preferably a hydrogen atom, halogen, —CN, —C(═O)—OH, —C(═O)—R¹⁵ or —C(═O)—OR¹⁵; R¹⁵ is each independently lower alkyl or halo lower alkyl. More preferably, R¹² is a hydrogen atom, a fluorine atom, a chlorine atom or —CN.

R¹¹ is preferably carboxylate anion (—COO⁻) or the group represented as follows:

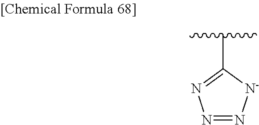 [SMILES: *c1nnn[n-]1]

### Formula (I):

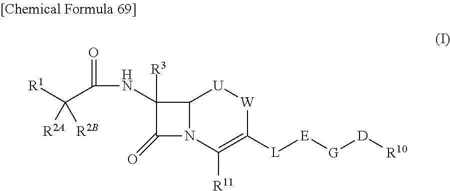

is preferably Formula (I-1):

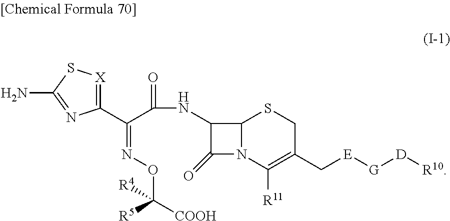

Preferred embodiments of Formula (I-1) are exemplified as follows. The compounds represented by embodiments 1 to 3 are exemplified as all possible combinations of these illustrative embodiments.

### Embodiment 1

### X is —N═, —CH═ or —CCl═,

R⁴ is a hydrogen atom, methyl or carboxy methyl, R⁵ is a hydrogen atom, methyl or carboxy methyl,
R¹¹ is carboxylate anion or a group represented by Formula:

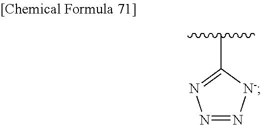

E is a group selected from the group consisting of the above Formulae (1), (2), (5), (7), (10), (11), (26) to (29), (31) and (41),
G is a single bond, methylene or ethylene,
D is —C(═O)—C(═O)—NR⁶—, —NR⁶—C(═O)—C(═O)—, —C(═O)—NR⁶—C(═O)—, —NR⁶—C(═O)—C(═N—OR⁶)—, —C(═O)—C(═N—OR⁶)— or —C(═O)—C(═O)—,
R⁶ is each independently a hydrogen atom, methyl, ethyl, 1-carboxy ethyl or 2-carboxypropane-2-yl,
R¹⁰ is substituted phenyl or substituted pyridyl, and which have at least two hydroxyl groups which bind to each of adjacent ring member atoms.

### Embodiment 2

### X is —N═, —CH═ or —CCl═,

R⁴ is a hydrogen atom, methyl or carboxy methyl, R⁵ is a hydrogen atom, methyl or carboxymethyl,
R¹¹ is carboxylate anion or a group represented by Formula:

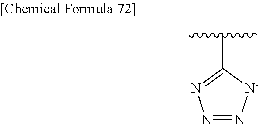 [SMILES: *c1nnn[n-]1]

E is a group selected from the group consisting of the above Formulae (1), (2), (5), (7), (10), (11), (26) to (29), (31) and (41),
G is a single bond, methylene or ethylene,
D is a single bond, —C(═O)—C(═O)—NR⁶—, —NR⁶—C(═O)—C(═O)—, —C(═O)—NR⁶—C(═O)—, —NR⁶—C(═O)—C(═N—OR⁶)—, —C(═O)—C(═N—OR⁶)— or —C(═O)—C(═O)—,
R¹⁰ is substituted benzisoxazolyl or substituted benzimidazolyl, and the groups which have at least two hydroxyl groups which bind to each of adjacent ring member atoms.

### Embodiment 3

### X is —N═, —CH═ or —CCl═,

R⁴ is a hydrogen atom, methyl or carboxy methyl, R⁵ is a hydrogen atom, methyl or carboxymethyl,
R¹¹ is carboxylate anion or a group represented by Formula:

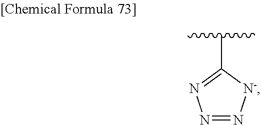

E is a group selected from the group consisting of the above Formulae (1), (2), (5), (7), (10), (11), (26) to (29), (31) and (41),
G is a single bond, methylene or ethylene,
D is a single bond, —C(═O)—C(═O)—NR⁶—, —NR⁶—C(═O)—C(═O)—, —C(═O)—NR⁶—C(═O)—, —NR⁶—C(═O)—C(═N—OR⁶)—, —C(═O)—C(═N—OR⁶)— or —C(═O)—C(═O)—,
R¹⁰ is a group represented by Formula:

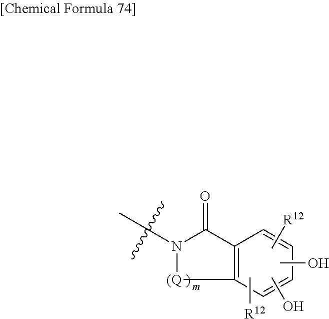

wherein m is 1 or 2,
when m is 1, Q is —O—, —S—, —NR⁸—, —CR⁸R⁹—, —C(═O)— or —N═CH—,
when m is 2, Q is each independently —O—, —S—, —NR⁸— or —C(═O)—, R⁸ and R⁹ are each independently a hydrogen atom, methyl, ethyl or trifluoromethyl, R¹² is each independently a hydrogen atom, halogen, hydroxy, —CN— or OR¹⁵, R¹⁵ is methyl, ethyl, isopropyl or trifluroromethyl.

The nomenclature of the substitution position on the Cephem skeleton of Formula (I) is as follows. As used herein, 3-side chain, 4-side chain and 7-side chain respectively refer to groups binding to the 3-position, 4-position and the 7-position of the Cephem skeleton as shown below:

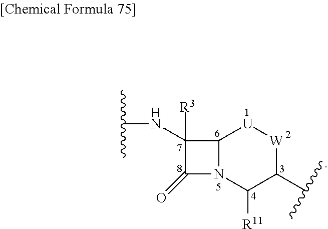

Esters of Formula (I) preferably include those esters at the 7-side chain. Esters at the carboxyl group on the 7-side chain include compounds having a structure in which the carboxyl group of a substituted or unsubstituted amino group, substituted or unsubstituted aminosulfonyl group, carboxyl group, substituted or unsubstituted lower alkyloxycarbonyl group, substituted or unsubstituted carbamoyl group, substituted carbonyloxy group, or the like at the terminal of R¹, R^{2A} or R^{2B} shown in Formula:

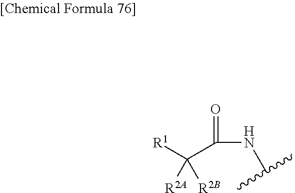

wherein each symbol is as defined above, is esterified (for example, in the case of carboxy (—COOH), such esters are represented by the structural formula —COOR^{a}, in which is shown with R^{a} representing an ester residue such as a carboxyl-protecting group or the like); and the like. Such esters include those esters that are easily metabolized in the body to form a carboxylic state.

The aforementioned protecting groups for a carboxyl group or the like may be of any group as long as it can be protected and/or deprotected by a method descried in Protective Groups in Organic Synthesis, written by T. W. Greene, John Wiley & Sons Inc. (1991), or the like. Examples thereof include lower alkyl (e.g., methyl, ethyl, t-butyl), lower alkylcarbonyloxymethyl (e.g. pivaloyl), substituted or substituted arylalkyl (e.g., benzyl, benzhydryl, phenethyl, p-methoxybenzyl, p-nitrobenzyl), silyl groups (e.g., t-butyldimethylsilyl, diphenyl t-butylsilyl), and the like.

Amino-protected compounds at the amino on the 7-side chain of Formula (I) refer to the structures in which the amino on the ring has been protected, as shown in Formula:

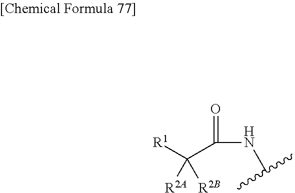

wherein each symbol is as defined above; and when R¹ and/or R^{2A} has an amino group, the protected compound is represented by Formula: —NHR^{c} wherein R^{e} represents an amino-protecting group. Such amino-protecting groups include those groups that are readily metabolized in the body to form amino. The aforementioned amino-protecting groups may be of any group as long as it can be protected and/or deprotected by a method described in Protective Groups in Organic Synthesis, written by T. W. Greene, John Wiley & Sons Inc. (1991), or the like. Examples thereof include lower alkyloxycarbonyl (e.g., t-butoxycarbonyl, benzyloxycarbonyl, p-nitrobenzyloxycarbonyl), optionally substituted aralkanoyl (e.g., benzoyl, p-nitrobenzoyl), acyl (e.g, formyl, chloroacetyl), and the like.

The Compound (I) of the subject invention is not limited to particular isomers, but includes any possible isomers (e.g., keto-enol isomer, imine-enamine isomer, diastereoisomer, optical isomer, rotamer, etc.), racemates and a mixture thereof.

For example,

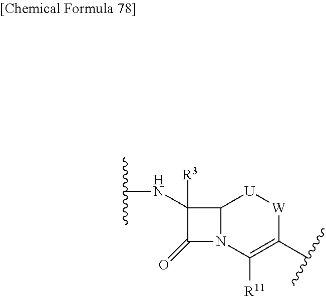

in Formula (I) includes

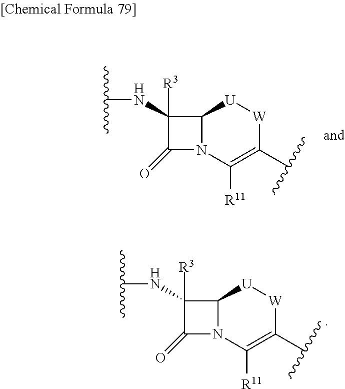

The compound (I) of the subject invention can form a zwitter ion between a quaternary ammonium ion on the group “E” and a substituent on the 4-side chain (i.e., a bioisoster of —COO⁻). For example, when the substituent at the 4-position is tetrazolyl group:

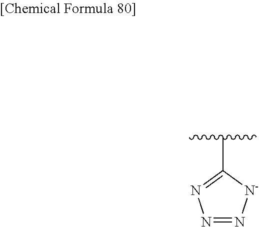 [SMILES: *c1nnn[n-]1]

which is negatively charged, but it may take the structure

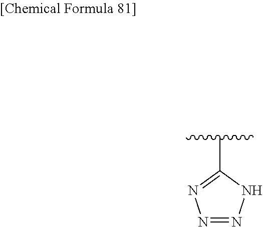 [SMILES: *c1nnn[nH]1]

by receiving a proton from another moiety in Formula (I), and such structure should be included in the compound (I) of the subject invention. The same is true in another bioisoster of carboxylate anion (—COO⁻).

Also, the group “E” in Formula (I), for example, includes the following resonance structures:

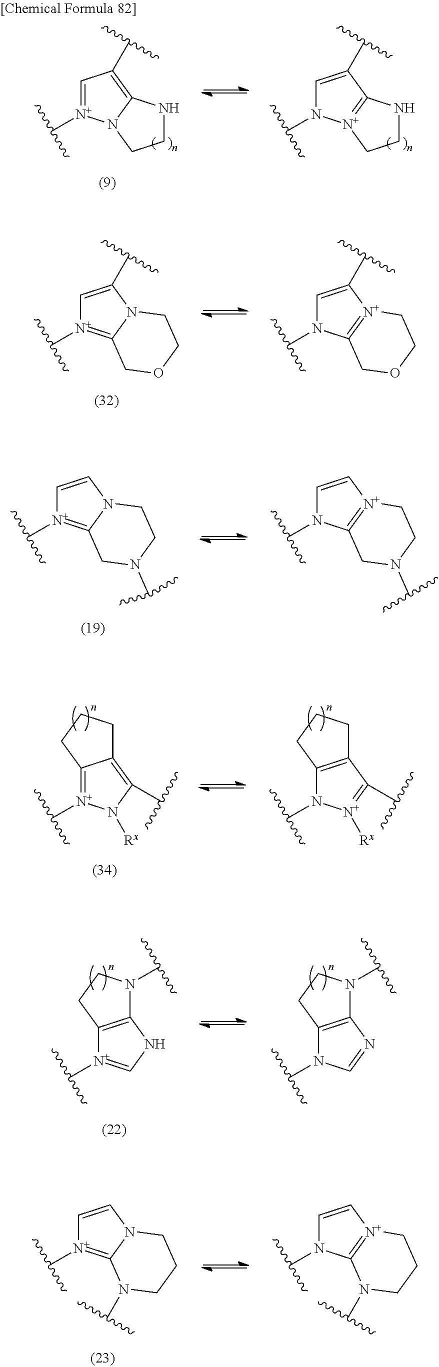

wherein each symbol is as defined above.

At least one hydrogen atom, carbon and/or another atom may be replaced with an isotope of said hydrogen, carbon and/or another atom. Examples of such isotope include hydrogen, carbon, nitrogen, oxygen sulfur, fluorine, iodine and chlorine, such as ²H, ³H, ¹¹C, ³C, ¹⁴C, ¹⁵N, ¹⁸O, ¹⁷O, ³¹P, ³²P, ³⁵S, ¹⁸F, ¹²³I and ³⁶Cl. The compound of Formula (I) include compounds having an atom replaced with such isotope. Such compounds replaced with an isotope are useful as a pharmaceutical product, and such compound include all of radiolabeled compound of Formula (I). Also, the subject invention includes any method of radioactive labeling for the production of such radiolabeled compound, and thus, it is useful in a research for metabolic pharmacokinetics, binding assay and/or as a diagnostic tool.

A radiolabeled compound of Formula (I) may be prepared according to the technique well known in the art. For example, tritium can be introduced into a specific compound of Formula (I) by catalytic dehalogenation using tritium to prepare a tritium-labeled compound of Formula (I). This method comprises reaction of a precursor which is a compound of Formula (I) appropriately halogenated with tritium gas in the presence of appropriate catalyst, such as Pd/C, in presence or absence of a base. For another method for the preparation of a tritium-labeled compound, see the literature, Isotopes in the Physical and Biomedical Sciences, Vol. 1, Labeled Compounds (Part A), Chapter 6 (1987). ¹⁴C-labeled compound can be prepared using a starting material having ¹⁴C.

Salts of a compound of Formula (I) include those formed with an inorganic or organic acid by a carboxyl group in the 7-side chain and/or an amino group in the 7-side chain; and those formed with a counter anion by a quaternary amine moiety in the 3-side chain.

Pharmaceutically acceptable salts of a compound of Formula (I) include, for example, salts formed with alkali meta (e.g., lithium, sodium, potassium, etc.), alkaline earth metal (e.g., calcium, barium, etc.), magnesium, transition metal (e.g., zinc, ferrum, etc.), ammonia, organic base (e.g., trimethylamine, triethylamine, dicyclohexylamine, ethanolamine, diethanolamine, triethanolamine, meglumine, diethanolamine, ethylenediamine, pyridine, picoline, quinolone, etc.), and amino acid, or salts formed with inorganic acid (e.g., hydrochloric acid, sulphuric acid, nitric acid, carbonic acid, hydrobromic acid, phosphoric acid, hydroiodic acid, etc.), and organic acid (e.g., formic acid, acetic acid, propionic acid, trifluoroacetic acid, citric acid, lactic acid, tartaric acid, oxalic acid, maleic acid, fumaric acid, mandelic acid, glutaric acid, malic acid, benzoic acid, phthalic acid, ascorbic acid, benzenesulphonic acid, p-toluenesulfonic acid, methanesulphonic acid, ethanesulphonic acid, etc, particularly, salts formed with hydrochloric acid, sulphuric acid, phosphoric acid, tartric acid, methanesulphonic acid. These salts can be formed according to the conventional method.

The compound of Formula (I) or pharmaceutically acceptable salts thereof may form a solvate (e.g., hydrate etc.) and/or a crystalline polymorphism, and the subject invention also includes such solvates and crystalline polymorphisms. In such “solvate”, any number of solvent molecules (e.g., water molecule, etc.) may be coordinated to the compound of Formula (I). By leaving the compound of Formula (I) or pharmaceutically acceptable salt thereof in the atmosphere, it may absorb moisture to adhere with absorbed water or form a hydrate thereof. Also, a crystalline polymorphism of the compound of Formula (I) or pharmaceutically acceptable salt thereof can be formed by recrystallization.

The compound of Formula (I) or pharmaceutically acceptable salt thereof may form a prodrug, and the subject invention includes such prodrugs. Prodrug is a derivative of the compound of the invention having a group chemically- or metabolically-degradable to be transformed into a pharmacologically active compound by solvolysis or under physiological condition in vivo. Prodrug includes compounds which can be transformed into the compound of Formula (I) by enzymatically oxidization, reduction or hydrolysis under physiological condition in vivo, or transformed into the compound of Formula (I) by hydrolysis with gastric acid, etc. Methods for selection and production of appropriate prodrug derivative can be found, for example, in Design of Prodrugs, Elsevier, Amsterdam 1985. Prodrug may be active compound in itself.

When the compound of Formula (I) or pharmaceutically acceptable salt thereof has a hydroxyl group, acyloxy derivatives or sulfonyloxy derivatives can be prepared as a prodrug. For example, such compound having a hydrozyl group may be reacted with an appropriated acyl halide, acid anhydrate, mixed anhydrate, etc., or may be reacted using a coupling agent, such as for examples, those having CH₃COO—, C₂H₅COO—, t-BuCOO—, C₁₅H₃₁COO—, PhCOO—, (m-NaOOCPh)COO—, NaOOCCH₂CH₂COO—, CH₃CH(NH₂)COO—, CH₂N(CH₃)₂COO—, CH₃SO₃—, CH₃CH₂SO₃—, CF₃SO₃—, CH₂FSO₃—, CF₃CH₂SO₃—, p-CH₃—O-PhSO₃—, PhSO₃—, p-CH₃PhSO₃—.

For the synthesis of a compound of Formula (I), a compound represented by Formula:

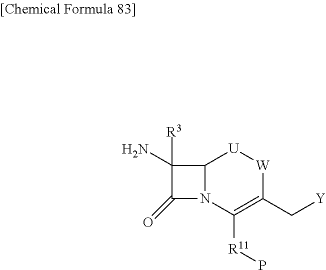

wherein Y is a leaving group, U, W, R³ and R¹¹ are as defined above, P is a protecting group as defined above, or a pharmaceutically acceptable salt thereof is preferred as an intermediate.

The leaving group includes halogen (Cl, Br, I, F), methanesulfonyloxy, p-toluenesulfonyloxy, trifluoromethanesulfonyloxy, and the like.

As described in the following General Synthesis and Examples, an intermediate compound described above is attached with side chain moieties at the 3-, 4- and 7-positions of the cephem skeleton to obtain a compound of Formula (I). Examples of the protecting group “P” include those described in the following General Synthesis, and preferably, benzhydryl group, p-methoxybenzyl group, trityl group, 2,6-dimethoxybenzyl group, methoxymethyl group, benzyloxymethyl group or 2-(trimethylsilyl)ethoxymethyl group, etc.

### (General Synthesis Method)

The compounds represented by Formula (I) of the subject invention can be manufactured, for example, by a general synthesis method described below:

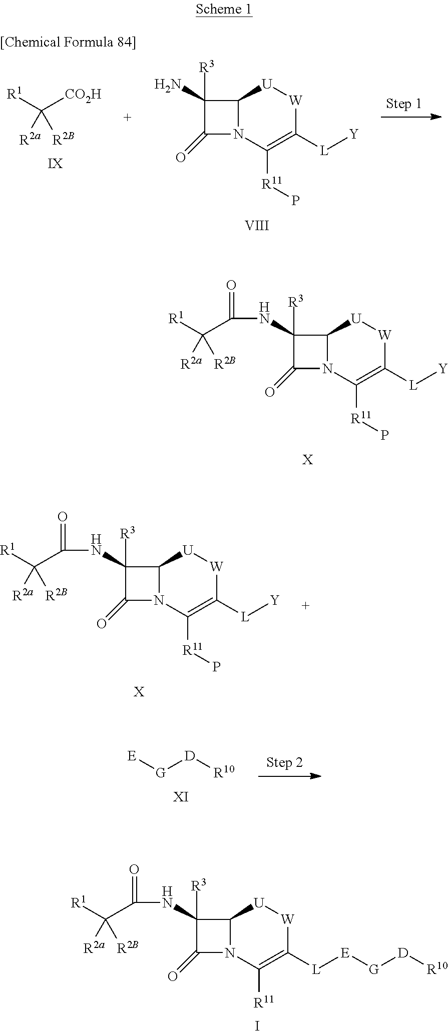

wherein W, U, R¹, R^{2A}, R^{2B}, R³, R¹⁰, R¹¹, L, E, G and D are as defined above, P is a protecting group, Y is a leaving group (e.g., halogen (Cl, Br, I, F), methanesulfonyloxy, p-toluenesulfonyloxy, trifluoromethanesulfonyloxy, etc.

### 1) Formation of 7-Side Chain: Synthesis of Compound (X)

### Step 1

Compound (X) may be obtained by subjecting Compound (VIII) to a condensation reaction with Compound (IX). The reaction solvents include, for example, ethers (e.g., anisole, dioxane, tetrahydrofuran, diethylether, tert-butyl methyl ether, diisopropylether), esters (e.g., ethyl formate, ethyl acetate, n-butyl acetate), halogenated hydrocarbons (e.g., dichloromethane, chloroform, carbon tetrachloride), hydrocarbons (e.g., n-hexane, benzene, toluene), amides (e.g., formamide, N,N-dimethylformamide, N,N-dimethylacetamide, N-methylpyrrolidone), ketones (e.g., acetone, methyl ethyl ketone), nitriles (e.g., MeCN, propionitrile), nitros (e.g., nitromethane, nitroethane, nitrobenzene), dimethylsulfoxide, and water, or a mixed solvent selected from two or more of these solvents. The reaction temperature is usually in the range of from about −100° C. to 100° C., preferably in the range from about −80° C. to 20° C., more preferably in the range of from about −60° C. to −20° C. The reaction time may vary according to the reagents, solvents or reaction temperature to be employed, and usually, is 0.5 to 24 hours.

### 2) Formation of the 3-Side Chain: Synthesis of Compound (I)

### Step 2

Compound (I) may be obtained by reacting Compound (X) with Compound (XI), followed by deprotecting by a method well-known to those skilled in the art. The reaction solvents include, for example, ethers (e.g., anisole, dioxane, tetrahydrofuran, diethylether, tert-butyl methyl ether, diisopropylether), esters (e.g., ethyl formate, ethyl acetate, n-butyl acetate), halogenated hydrocarbons (e.g., dichloromethane, chloroform, carbon tetrachloride), hydrocarbons (e.g., n-hexane, benzene, toluene), amides (e.g., formamide, N,N-dimethylformamide, N,N-dimethylacetamide, N-methylpyrrolidone), ketones (e.g., acetone, methyl ethyl ketone), nitriles (e.g., MeCN, propionitrile), nitros (e.g., nitromethane, nitroethane, nitrobenzene), dimethylsulfoxide, and water, or a mixed solvent selected from two or more of these solvents. The reaction temperature is usually in the range of from about −100° C. to 100° C., preferably in the range of from about −80° C. to 50° C., more preferably in the range of from −40 to 0° C. The reaction time may vary according to the reagents, solvents or reaction temperature to be employed, and usually, is 0.5 to 24 hours.

The compounds represented by Formula (I) which have carboxylate anion (—COO⁻) at the 4-position of Cephem can be manufactured, for example, by a general synthesis method described below:

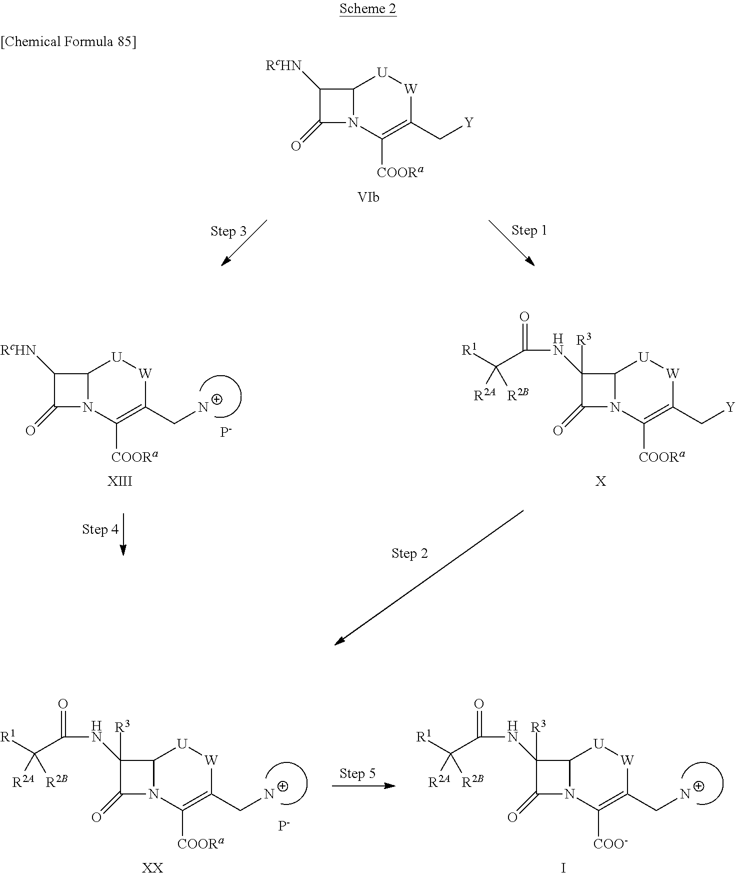

wherein R^{a} is a hydrogen atom or carboxy protecting group, R^{c} is a hydrogen atom or amino protecting group, U, W, L, R¹, R^{2A}, R^{2B} and R³ are as defined above, P⁻ is a counter anion of the quaternary ammonium ion (halogen etc.), Y is a leaving group (e.g., halogen (Cl, Br, I, F), methanesulfonyloxy, p-toluenesulfonyloxy, trifluoromethanesulfonyloxy, etc.), the group represented by Formula

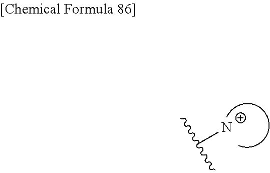 [SMILES: *[NH3+]]

represents the following moiety in Formula (I) including a quaternary ammonium group moiety of 3-side chain of Cephem:

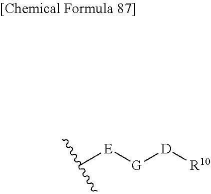

wherein each symbol is as defined above.

### 1) 7-Amidation and Formation of the 3-Side Chain; Synthesis of Compound (XX)

### Step 1 (7-Amidation Reaction):

Compound (C) is obtained by reacting Compound (VIb), which is commercially available or synthesized according to methods described in a document (e.g., JP 60-231684, JP 62-149682, etc.), and the compound represented by Formula corresponding to a desired side-chain:

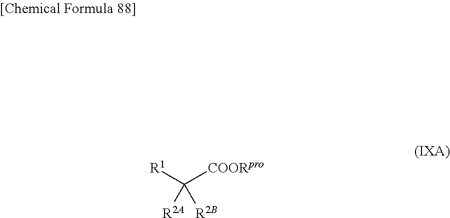

wherein R^{pro} is a hydrogen atom or a carboxy protecting group, the other symbols are as defined above. In this case, preferably, R^{a} is a carboxy protecting group, and R^{pro} is a hydrogen atom. The compound represented by Formula (IXA) can be obtained by using a commercially available reagent and/or a well-known method.

The amount of Compound (IVA) used is in a range of, generally, about 1 to 5 moles, preferably 1 to 2 moles, relative to 1 mole of Compound (VIb).

The reaction solvent include, for example, ethers (e.g., anisole, dioxane, tetrahydrofuran, diethylether, tert-butyl methyl ether, diisopropylether), esters (e.g., ethyl formate, ethyl acetate, n-butyl acetate), halogenated hydrocarbons (e.g., dichloromethane, chloroform, carbon tetrachloride), hydrocarbons (e.g., n-hexane, benzene, toluene), amides (e.g., formamide, N,N-dimethylformamide, N,N-dimethylacetamide, N-methylpyrrolidone), ketones (e.g., acetone, methyl ethyl ketone), nitriles (e.g., MeCN, propionitrile), nitros (e.g., nitromethane, nitroethane, nitrobenzene), dimethylsulfoxide, water, and the like, and mixed solvents thereof and the like.

The reaction temperature is in a range of, generally, about −40 to 80° C., preferably about −20 to 50° C., more preferably about −10 to 30° C.

The above-described amidation reaction may be carried out after a carboxy moiety is converted to a reactive derivative (e.g., inorganic base salt, organic base salt, acid halide, acid azide, acid anhydride, mixed acid anhydride, active amide, active ester, and active thioester). Examples of such inorganic bases include alkali metal (e.g., Na, K, etc.), alkali earth metal (e.g., Ca, Mg), and the like. Examples of organic bases include trimethylamine, triethylamine, tert-butyldimethylamine, dibenzylmethylamine, benzyldimethylamine, N-methylmorpholine, diisopropylethylamine, and the like. Examples of acid halides include acid chlorides, acid bromides, and the like. Examples of mixed acid anhydrides include mixed acid anhydrides of mono-alkyl carbonates, mixed acid anhydrides of aliphatic carboxylic acid, mixed acid anhydrides of aromatic carboxylic acid, mixed acid anhydrides of organic sulfonic acid, and the like. Examples of active amides include amides with nitrogen-containing heterocyclic compound, and the like. Examples of active esters include organic phosphoric esters (e.g., diethoxyphosphoric ester, diphenoxyphosphoric ester, and the like), p-nitrophenyl ester, 2,4-dinitrophenyl ester, cyanomethyl ester, and the like. Examples of active thioesters include esters with aromatic heterocyclic thiol compound (e.g., 2-pyridylthiol esters), and the like. Furthermore, in the above-described reaction, a suitable condensing agent may be used as desired. For example, hydrochloric acid salt of 1-dimethylaminopropyl-3-ethylcarbodiimide (WSCD.HCl), N,N′-dicyclohexylcarbodiimide, N,N′-carbonyldiimidazole, N,N′-thiocarbonyldiimidazole, N-ethoxycarbonyl-2-ethoxy-1,2-dihydroquinoline, phosphorus oxychloride, alkoxyacetylene, 2-chloromethylpyridinium iodide, 2-fluoromethylpyridinium iodide, trifluoroacetic anhydride, and the like can be used as a condensing agent.

### Step 2 (3-Side Chain Forming Reaction):

Compound (XX) is obtained by reacting Compound (X) and a corresponding tertiary amine. In the case, preferably, R^{a} is carboxy protecting group.

The amount of a corresponding tertiary amine used is in a range of, generally, 1 to 5 moles, preferably 1 to 2 moles, relative to 1 mole of Compound (X).

The reaction solvent include, for example, ethers (e.g., anisole, dioxane, tetrahydrofuran, diethylether, tert-butyl methyl ether, diisopropylether), esters (e.g., ethyl formate, ethyl acetate, n-butyl acetate), halogenated hydrocarbons (e.g., dichloromethane, chloroform, carbon tetrachloride), hydrocarbons (e.g., n-hexane, benzene, toluene), amides (e.g., formamide, N,N-dimethylformamide, N,N-dimethylacetamide, N-methylpyrrolidone), ketones (e.g., acetone, methyl ethyl ketone), nitriles (e.g., MeCN, propionitrile), dimethylsulfoxide, water, and the like, and mixed solvents thereof.

Furthermore, Compound (XX) wherein U is S can be obtained by reducing Compound (XX) wherein U is S(═O). Examples of reducing agents include potassium iodide-acetyl chloride, and the like.

### 3) 3-Side Chain Formation and 7-Amidation; Synthesis of Compound (XX)

### Step 3 (3-Side Chain Forming Reaction):

Compound (XIII) is obtained by reacting Compound (VIb) and a corresponding tertiary amine. In this case, preferably, R^{a} is a carboxy protecting group, and R^{c} is an amino protecting group.

The amount of a corresponding tertiary amine used in a range of, generally, 1 to 5 moles, preferably 1 to 2 moles, relative to 1 mole of Compound (VIb).

The reaction solvent include, for example, ethers (e.g., anisole, dioxane, tetrahydrofuran, diethylether, tert-butyl methyl ether, diisopropylether), esters (e.g., ethyl formate, ethyl acetate, n-butyl acetate), halogenated hydrocarbons (e.g., dichloromethane, chloroform, carbon tetrachloride), hydrocarbons (e.g., n-hexane, benzene, toluene), amides (e.g., formamide, N,N-dimethylformamide, N,N-dimethylacetamide, N-methylpyrrolidone), ketones (e.g., acetone, methyl ethyl ketone), nitriles (e.g., MeCN, propionitrile), dimethylsulfoxide, water, and the like, and mixed solvents thereof.

The reaction temperature is in a range of, generally, −20 to 60° C., preferably −10 to 40° C., more preferably 0 to 20° C.

Moreover, both tertiary amine moieties used in the 3-side chain forming reactions of Step 2 and Step 3 (corresponding to the moiety E in Item 1) can be obtained as a commercially available reagent, by a known method, and/or by a method described herein.

### Step 4 (7-Amidation Reaction):

Compound (XX) is obtained by reacting Compound (XIII) and Compound (IXA). In this case, preferably R^{a} is a carboxy protecting group, R^{c} is an amino protecting group, and R^{pro} and R^{e} are hydrogen atoms.

The amount of Compound (IXA) used is in a range of, generally, about 1 to 5 moles, preferably 1 to 2 moles.

The reaction solvent include, for example, ethers (e.g., anisole, dioxane, tetrahydrofuran, diethylether, tert-butyl methyl ether, diisopropylether), esters (e.g., ethyl formate, ethyl acetate, n-butyl acetate), halogenated hydrocarbons (e.g., dichloromethane, chloroform, carbon tetrachloride), hydrocarbons (e.g., n-hexane, benzene, toluene), amides (e.g., formamide, N,N-dimethylformamide, N,N-dimethylacetamide, N-methylpyrrolidone), ketones (e.g., acetone, methyl ethyl ketone), nitriles (e.g., MeCN, propionitrile), dimethylsulfoxide, water, and the like, and mixed solvents thereof.

The reaction temperature is in a range of, generally, about −40 to 80° C., preferably about −20 to 50° C., more preferably about −10 to 30° C.

The above-described amidation reaction may be carried out after a carboxyl moiety is converted to a reactive derivative (e.g., inorganic base salt, organic base salt, acid halide, acid azide, acid anhydride, mixed acid anhydride, active amide, active ester, and active thioester). Examples of such inorganic bases include alkali metal (e.g., Na, K, etc.), alkali earth metal (e.g., Ca, Mg), and the like. Examples of organic bases include trimethylamine, triethylamine, tert-butyldimethylamine, dibenzylmethylamine, benzyldimethylamine, N-methylmorpholine, diisopropylethylamine, and the like. Examples of acid halides include acid chlorides, acid bromides, and the like. Examples of mixed acid anhydrides include mixed acid anhydrides of mono-alkyl carbonate, mixed acid anhydrides of aliphatic carboxylic acid, mixed acid anhydrides of aromatic carboxylic acid, mixed acid anhydrides of organic sulfonic acid, and the like. Examples of active amides include amides with nitrogen-containing heterocyclic compound, and the like. Examples of active esters include organic phosphoric esters (e.g., diethoxyphosphoric ester, diphenoxyphosphoric ester, and the like), p-nitrophenyl ester, 2,4-dinitrophenyl ester, cyanomethyl ester, and the like. Examples of active thioesters include esters with aromatic heterocyclic thiol compound (e.g., 2-pyridylthiol esters), and the like. Examples of active thioesters include esters with aromatic heterocyclic thiol compound (e.g., 2-pyridylthiol esters), and the like. Furthermore, in the above-described reaction, a suitable condensing agent may be used as desired. For example, hydrochloric acid salt of 1-dimethylaminopropyl-3-ethylcarbodiimide (WSCD.HCl), N,N′-dicyclohexylcarbodiimide, N,N′-carbonyldiimidazole, N,N′-thiocarbonyldiimidazole, N-ethoxycarbonyl-2-ethoxy-1,2-dihydroquinoline, phosphorus oxychloride, alkoxyacetylene, 2-chloromethylpyridinium iodide, 2-fluoromethylpyridinium iodide, trifluoroacetic anhydride, and the like can be used as a condensing agent.

Furthermore, Compound (XX) wherein U is O can be obtained using Compound (VIb) wherein U is O.

### 4) Deprotection Reaction:

### Step 5:

Compound (I) is obtained by subjecting Compound (XX) to a deprotection reaction according to a method well known to those skilled in the art.

The reaction solvent include, for example, ethers (e.g., anisole, dioxane, tetrahydrofuran, diethylether, tert-butyl methyl ether, diisopropylether), esters (e.g., ethyl formate, ethyl acetate, n-butyl acetate), halogenated hydrocarbons (e.g., dichloromethane, chloroform, carbon tetrachloride), hydrocarbons (e.g., n-hexane, benzene, toluene), amides (e.g., formamide, N,N-dimethylformamide, N,N-dimethylacetamide, N-methylpyrrolidone), ketones (e.g., acetone, methyl ethyl ketone), nitriles (e.g., MeCN, propionitrile), nitros (e.g., nitromethane, nitroethane, nitrobenzene), dimethylsulfoxide, and water, or a mixed solvent selected from two or more of these solvents.

The reaction temperature is in a range of, generally, about −30 to 100° C., preferably about 0 to 50° C., more preferably about 0 to 10° C.

As a catalyst, Lewis acid (e.g., AlCl₃, SnCl₄, TiCl₄), protonic acid (e.g., HCl, HBr, H₂SO₄, HCOOH), and the like can be used.

Furthermore, the obtained compound (I) is further chemically modified to obtain an ester, or a compound wherein the amino on the thiazole ring at the 7-position is protected, or a pharmaceutically acceptable salt, or a solvate thereof.

The protecting group (amino protecting groups, hydroxyl protecting groups, etc.) include, for example, protecting groups desclibed in Protective Groups in Organic Synthesis, written by T. W. Green, John Wiley & Sons Inc. (1991), such as ethoxycarbonyl, t-butoxycarbonyl, acetyl, benzyl, and the like. Methods for the introduction and removal of a protecting group are methods commonly used in synthetic organic chemistry (see, for example, Protective Groups in Organic Synthesis, written by T. W. Greene, John Wiley & Sons Inc. (1991)), etc., or can be obtained group included in each substituent can be converted by a known method (e.g., those described in Comprehensive Organic Transformations, written by R. C. Larock (1989), etc.) in addition to the above production methods. Some of the compounds of the present invention can be used as a synthetic intermediate, leading to a new derivative. Intermediates and desired compounds produced in each of the above production methods can be isolated and purified by a purification method commonly used in synthetic organic chemistry, for example, neutralization, filtration extraction, washing, drying, concentration, recrystallization, any kind of chromatography and the like. Furthermore, intermediates can be subjected to a next reaction without any purification.

If Compound of Formula (I) has a tetrazol ring at 4-position Compound (VIII) used in the above Scheme 3 can be prepared according to Scheme 1.

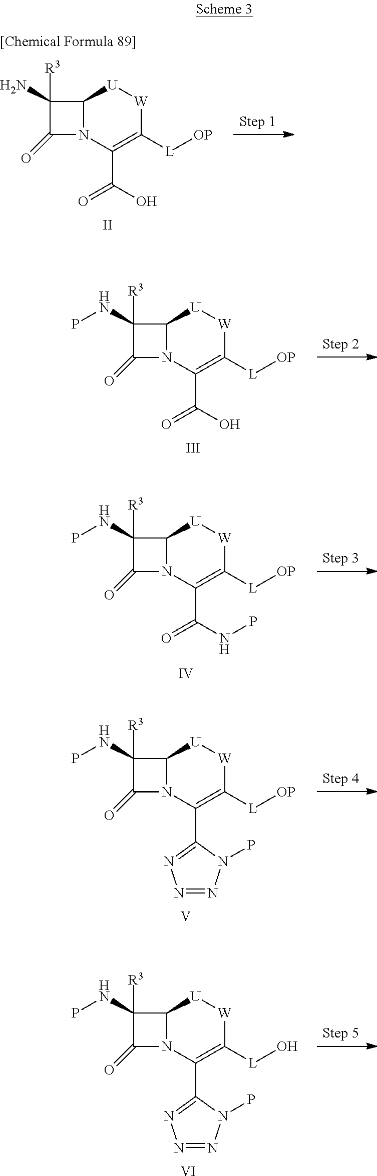

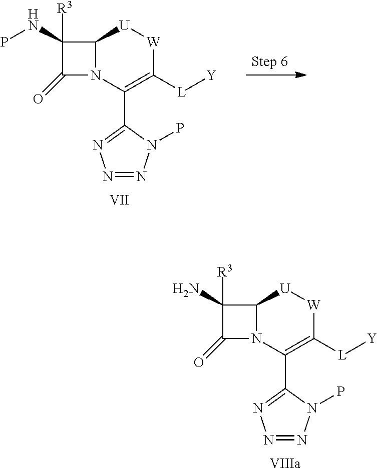

wherein W, U, R³ and L are as defined above, P is a protecting group, and Y is a leaving group (e.g., halogen (Cl, Br, I, F), methanesulfonyloxy, p-toluenesulfonyoxy, trifluoromethansulfonyloxy, and the like.

### Step 1

Compound (III) is obtained by protecting an amino group on the 7-side chain of Compound (II) with a protecting group by a method well-known to those skilled in the art. The protecting groups to be used include the amino-protecting groups as exemplified below.

### Step 2

Compound (IV) is obtained by amidation of a carboxyl group at 4-position of Compound (III) by a method well-known to those skilled in the art. This amidation may be carried out by using an amine compound which is previously protected by a protecting group, or alternatively, an amide group on the 4-side chain may be protected after the amidation. The protecting groups to be used include the amide-protecting groups as exemplified below. The reaction solvents include, for example, ethers (e.g., anisole, dioxane, tetrahydrofuran, diethylether, tert-butyl methyl ether, diisopropylether), esters (e.g., ethyl formate, ethyl acetate, n-butyl acetate), halogenated hydrocarbons (e.g., dichloromethane, chloroform, carbon tetrachloride), hydrocarbons (e.g., n-hexane, benzene, toluene), amides (e.g., formamide, N,N-dimethylformamide, N,N-dimethylacetamide, N-methylpyrrolidone), ketones (e.g., acetone, methyl ethyl ketone), nitriles (e.g., MeCN, propionitrile), dimethylsulfoxide, water, and the like, and mixed solvents thereof.

The reaction temperature is usually in a range of from about −100 to 100° C., preferably about −80 to 50° C., more preferably about −80 to −40° C. The reaction time may vary according to the reagents, solvents or reaction temperature to be employed, but usually is 0.5 to 24 hours.

### Step 3

Compound (V) is obtained by reacting Compound (IV) with, for example, hydrogen azide, trimethylsilyl azide (TMSN₃), hydrazoates (e.g.: sodium azide, tetra-n-butylammonium azide, tetramethylguanidinium azide), and the like to form a tetrazole ring.

The amount of trimethylsilyl azide used is in a range of, generally, about 1 to 100 moles, preferably 1 to 30 moles. The reaction solvents include, for example, water, alcohols (e.g., methanol, ethanol, etc.), carboxylic acids (e.g., acetic acid, etc.). The reaction temperature is usually in a range of from about 0 to 100° C., preferably about 10 to 90° C., more preferably about 10 to 50° C. The reaction time may vary according to the reagents, solvents or reaction temperature to be employed, but usually is 0.5 and 24 hours.

### Step 4

Compound (VI) is obtained by subjecting Compound (V) to a deprotection reaction by a method well-known to those skilled in the art.

### Step 5

Compound (VII) is obtained by halogenating a hydroxyl group on the 3-side chain of Compound (VI). The halogenating agents to be used include, for example, phosgene, triphosgene, and the like. The reaction solvents include, for example, ethers (e.g., anisole, dioxane, tetrahydrofuran, diethylether, tert-butyl methyl ether, diisopropylether), esters (e.g., ethyl formate, ethyl acetate, n-butyl acetate), halogenated hydrocarbons (e.g., dichloromethane, chloroform, carbon tetrachloride), hydrocarbons (e.g., n-hexane, benzene, toluene), amides (e.g., formamide, N,N-dimethylformamide, N,N-dimethylacetamide, N-methylpyrrolidone), ketones (e.g., acetone, methyl ethyl ketone), nitriles (e.g., MeCN, propionitrile), nitros (e.g., nitromethane, nitroethane, nitrobenzene), dimethylsulfoxide, and water, or a mixed solvent selected from two or more of these solvents. The reaction temperature is usually in a range from about −100 to 100° C., preferably about −80 to 50° C., more preferably about −20 to 30° C. The reaction time may vary according to the reagents, solvents or reaction temperature to be employed, but usually is 0.5 to 24 hours.

### Step 6

Compound (VIIIa) is subjected to deprotection of an amino-protecting group at 7-position by a metal well-known to those skilled in the art to obtain Compound (VII).

The protecting group to be used in the above reaction such as amino-protecting groups, hydroxy-protecting groups, etc., include, for example, protecting groups described in Protective Groups in Organic Synthesis, written by T. W. Greene, John Wiley & Sons Inc. (1991), etc. Methods for the introduction and removal of a protecting group are methods commonly used in synthetic organic chemistry (see, for example, methods described in Protective Groups in Organic Synthesis, written by T. W. Greene, John Wiley & Sons Inc. (1991)), etc., or can be obtained by a modified method thereof. Furthermore, a functional group included in each substituent can be converted by a known method (e.g., those described in Comprehensive Organic Transformations, written by R. C. Larock (1989), etc.) in addition to the above production methods. Some of the compounds of the present invention can be used as a synthetic intermediate, leading to a new derivative. Intermediates and desired compounds produced in each of the above production methods can be isolated and purified by a purification method commonly used in synthetic organic chemistry, for example, neutralization, filtration, extraction, any kind of chromatography, etc. Furthermore, intermediates can be subjected to a next reaction without any purification.

Examples of amino-protecting group include, for example, phthalimide, a lower alkoxycarbonyl (butoxycarbonyl (Boc), etc.), lower alkenyloxycarbonyl (allyloxycarbonyl (Alloc), etc.), benzyloxycarbonyl, p-nitrobenzyloxycarbonyl, (substituted) aralkanoyl (p-nitrobenzoyl, etc.), acyl (formyl, chloroacetyl, etc.), (substituted) arylalkyl (trityl, etc.), benzhydryl (BH), etc.

Examples of hydroxyl-protecting group include, for example, lower alkoxycarbonyl such as a C₁-C₄ alkyloxycarbonyl (e.g., t-butyloxycarbonyl), a halogenated lower alkyloxycarbonyl such as a halogenated (C₁-C₃)alkyloxycarbonyl (e.g., 2-iodo ethyloxycarbonyl, 2,2,2-trichloroethyloxycarbonyl), an aryl-lower alkoxycarbonyl such as a phenyl (C₁-C₄)alkyloxycarbonyl having optionally a substituent(s) on the benzene ring (benzyloxycarbonyl, o-nitrobenzyloxycarbonyl, p-nitrobenzyloxycarbonyl, p-methoxybenzyloxycarbonyl), p-methoxybenzyl (PMB), tri-lower alkylsilyl such as tri-(C₁-C₄)alkylsilyl(e.g., trimethylsilyl, t-butyldimethylsilyl), a substituted methyl such as a C₁-C₄ alkyloxymethyl (e.g., methoxymethyl), C₁-C₄ alkyloxy(C₁-C₄)alkyloxymethyl (e.g., 2-methoxyethoxymethyl), C₁-C₄ alkylthiomethyl (e.g., methylthiomethyl), tetrahydropyranyl, etc.

The above-mentioned deprotecting reaction is carried out in a solvent such satetrahydrofuran, dimethylformamide, diethylether, dichloromethane, toluene, benzene, xylene, cyclohexane, hexane, chloroform, ethyl acetate, butyl acetate, pentane, heptane, dioxane, acetone, acetonitrile, or a mixed solvent thereof, using a Lewis acid (e.g., AlCl₃, SnCl₄, TiCl₄), a protonic acid (e.g., HCl, HBr, H₂SO₄, HCOOH), etc.

The obtained compound is further chemically modified, and thereby an ester, or a compound of which an amino on the thiazole ring at the 7-position thereof is protected, or a pharmaceutically acceptable salt, or a solvate thereof can be synthesized.

The compounds of the present invention have a wide antimicrobial activity spectrum, and may be used for prevention or therapy against a variety of disease caused by causative bacteria in a variety of mammals including humans, for example, airway infectious diseases, urinary system infectious diseases, respiratory system infectious diseases, sepsis, nephritis, cholecystitis, oral cavity infectious diseases, endocarditis, pneumonia, bone marrow membrane myelitis, otitis media, enteritis, empyema, would infectious diseases, opportunistic infection, etc.

The compounds of the subject invention exhibit high antimicrobial activity in particular against Gram negative bacteria, preferably, Gram negative bacteria of enterobacteria (E. coli, Klebsiella, Serratia, Enterobacter; Citrobacter, Morganella, Providencia, Proteus and the like), Gram negative bacteria colonized in respiratory system (Haemophilus, Moraxella and the like), and Gram negative bacteria of glucose non-fermentable (Pseudomonas aeruginosa, Pseudomonas other than P. aeruginosa, Stenotrophomonas, Burkholderia, Acinetobacter and the like). The compounds are stable against beta-lactamase Class A, B, C and D which are produced by these Gram negative bacteria, and have high antimicrobial activity against a variety of beta-lactam drug resistant Gram negative bacteria, such as ESBL producing bacteria and the like. These are extremely stable against metallo-beta-lactamase belonging to Class B including in particular IMP type, VIM type, L-1 type and the like, and thus, these are effective against Gram negative bacteria resistant to a variety of beta-lactam drug including Cephem and Carbapenem. Still more preferable compounds have features regarding kinetics in the body, such as high blood concentration, long duration of effects, and/or significant tissue migration. More preferable compounds are safe in terms of side effects. Also, more preferable compounds have high water solubility, and thus particularly suitable for injectable formulations.

Compounds (I) may be administered parenterally or orally as injectable formulations, capsules, tablets, and granules, and preferably, administered as an injectable formulation. The dosage may usually be about 0.1 to 100 mg/day, preferably, about 0.5 to 50 mg/day, per 1 kg of body weight of a patient or animal, and optionally be divided into 2 to 4 times per day. The carriers for use in injectable formulation may be, for example, distilled water, saline and the like, and further bases may be used for pH adjustment. The carriers for used in capsules, granules or tablets, carriers include known excipients (for example, starch, lactose, sucrose, calcium carbonate, calcium phosphate and the like), binders (for example, starch, acacia gum, carboxymethyl cellulose, hydroxypropyl cellulose, crystalline cellulose, and the like), lubricants (for example, magnesium stearate, talc and the like), and the like.

### EXAMPLES

Hereinafter, the subject invention is described in more detail with working examples and experimental examples. However, the subject invention is not limited to them.

In the Examples, the meaning of each abbreviation is as described below.

Ac: acetyl
Alloc: allyoxy carbonyl
BH: benzhydryl
Boc: tert-butoxycarbonyl
DMA: N,N-dimethyl acetoamide

### DMF: N,N-dimethylformamide

EDC: 1-ethyl-3-(3-dimethylaminopropyl)carbodiimide
i-Pr: isopropyl
mCPBA: m-chloro peracetic acid
Me: methyl
ODS: octadodecylsilyl
PMB: p-methoxybenzyl
t-Bu: tert-butyl
TFA: trifluoroacetic acid
WSCD: N-ethyl-N-(3-dimethyl aminopropyl)carbodiimide

### Example 1

### Synthesis of Compound (I-1)

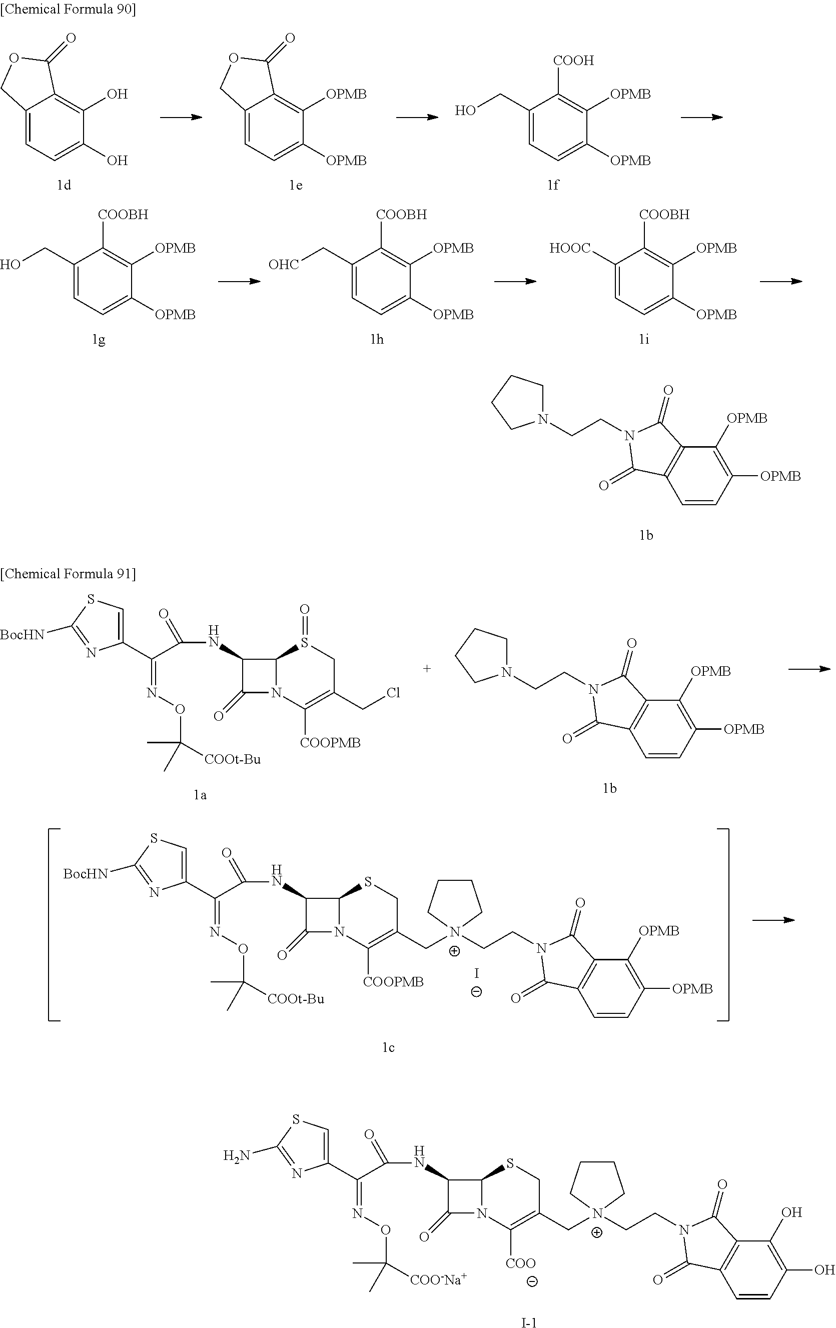

Step (1): Compound 1d→Compound 1e

Potassium carbonate (23.0 g, 166 mmol), p-methoxybenzyl chloride (22.7 mL, 166 mmol), and sodium iodide (5.67 g, 38 mmol) are added into the DMF solution (120 mL) of Compound 1d (12.6 g, 76 mmol), the solution was stirred at 70° C. for 1.5 hours. The solvent was removed under reduced pressure, then the obtained residue was added water, and extracted with ethyl acetate. The organic layer was washed with water, then saturated brine, and then dried with magnesium sulfate. The inorganic material was filtered out, and then the filtrate was concentrated. Diisopropylether was added to the resulting residue, and then the resulting solid was filtered to yield compound 1e (22.7 g, 74%) as a yellow solid.

¹H-NMR (CDCl₃) δ: 3.79 (3H, s), 3.82 (3H, s), 5.05 (2H, s), 5.17 (2H, s), 5.26 (2H, s), 6.82 (2H, d, J=8.5 Hz), 6.89 (2H, d, J=8.5 Hz), 7.00 (1H, d, J=8.2 Hz), 7.22 (1H, d, J=8.2 Hz), 7.30 (2H, d, J=8.2 Hz), 7.42 (2H, d, J=8.4 Hz).

Step (2): Compound 1e→Compound 1f

Compound 1e (22.4 g, 55 mmol) was dissolved in methanol (55 mL) and tetrahydrofuran (55 mL), 2 mmol/L sodium hydrate solution (83 mL, 165 mmol) was added thereto, and then the solution was stirred at 1.5° C. for 1.5 hours. After having cooled the reaction mixture to room temperature, diethylether was added thereto, and then the aqueous layer was separated. The aqueous layer was adjusted to pH=3.0 with 2 mol/L hydrochloric acid, the solution was extracted with dichloromethane. The organic layer was washed with water, and then saturated brine, and dried with anhydrous magnesium sulfate. The inorganic material was filtered out, and the filtrate was concentrated in vacuo, and the residue was dried in vacuo to yield compound 1f (20.5 g, 88%) as a pink solid.

¹H-NMR (CDCl₃) δ: 3.80 (3H, s), 3.84 (3H, s), 4.67 (2H, s), 5.11 (2H, s), 5.12 (2H, s), 6.82 (2H, d, J=8.7 Hz), 6.95 (2H, d, J=8.7 Hz), 7.19-7.24 (4H, m), 7.39 (2H, d, J=8.7 Hz).

Step (3): Compound 1f→Compound 1g→Compound 1h

A tetrahydrofuran solution (100 mL) of diphenyldiazomethane (23.0 g, 118 mmol) was added drop-wise to the tetrahydrofuran solution (350 mL) of compound 1f (45.7 g, 108 mmol) over 20 minutes, and then the solution was stirred at room temperature overnight. The solvent was removed in vacuo, and the residue was dried in vacuo to yield compound 1g as a yellow foam. The obtained compound 1g was used in the next reaction without any purification.

A solution of the whole amount of Compound 1g in dichloromethane (640 ml), Dess-Martin Periodinane (50.4 g, 119 mmol) was added thereto, and the resulting solution was stirred at 0° C. for 2 hours. The reaction mixture was added water, and dichloromethane was removed in vacuo, and then the residue was extracted with ethyl acetate. The organic layer was washed with water, then saturated brine, and then dried with anhydrous magnesium sulfate. The inorganic material was filtered out, and the filtrate was concentrated in vacuo. The resulting residue was added diisopropyl ether, and then the resulting solid was filtered to yield compound 1 h (51.1 g, 80%) as a white solid.

¹H-NMR (CDCl₃) δ: 3.79 (3H, s), 3.84 (3H, s), 4.86 (2H, s), 5.14 (2H, s), 6.68 (2H, d, J=8.66 Hz), 6.93 (2H, d, J=8.78 Hz), 6.97 (2H, d, J=8.66 Hz), 7.14 (1H, d, J=8.41 Hz), 7.19 (1H, s), 7.25-7.27 (5H, m), 7.36-7.38 (7H, m), 7.61 (1H, d, J=8.41 Hz), 9.74 (1H, s).

Step (4): Compound 1h→Compound 1i

Compound 1h (51.1 g, 87 mmol) was dissolved with 1,4-dioxane (600 ml) and water (200 ml), and after having cooled the resulting solution to 0° C., amide sulfate (16.9 g, 174 mmol) and sodium chlorite (19.6 g, 174 mmol) was added thereto in sequence, and stirred at 0° C. for 30 minutes. After the reaction mixture was slowly added a solution of sodium hydrogen sulfite (36.2 g, 348 mmol), the solution was extracted with ethyl acetate. The organic layer was washed with water and saturated brine in sequence, and then dried with anhydrous magnesium sulfate. The inorganic material was filtered out, and the filtrate was concentrated in vacuo. The resulting residue was added diisopropyl ether, and the resulting solid was filtered to yield compound 1i (51.4 g, 98%) as a white solid.

¹H-NMR (DMSO-D₆) δ: 3.73 (3H, s), 3.77 (3H, s), 4.71 (2H, s), 5.20 (2H, s), 6.69 (2H, d, J=8.59 Hz), 6.85 (2H, d, J=8.59 Hz), 6.97-6.99 (3H, m), 7.26-7.28 (6H, m), 7.36-7.38 (5H, m), 7.45 (2H, d, J=8.59 Hz), 7.75 (1H, d, J=8.84 Hz).

Step (5): Compound 1i→Compound 1b

After a solution of compound 1i (9.07 g, 15 mmol) in dimethylformamide (90 mL) was cooled to 0° C., 1-hydroxybenzotriazole (2.23 g, 16.5 mmol), 1-(2-aminoethyl)pyrrolidine (2.26 mL, 18 mmol), and EDC hydrochloride salt (3.74 g, 19.5 mmol) were added thereto in sequence, and stirred at room temperature for three and half hours. After having removed dimethylformamide in vacuo, the residue was added water, and extracted with ethyl acetate. The organic layer was washed 1 mol/L sodium hydrate solution, water and saturated brine in sequence, and then dried with anhydrous magnesium sulfate. The inorganic material was filtered out, and the filtrate was concentrated in vacuo. The resulting residue was added diisopropyl ether, and the resulting solid was filtered to yield compound 1b (6.73 g, 87%) as a white solid.

¹H-NMR (CDCl₃) δ: 1.74-1.77 (4H, m), 2.57-2.60 (4H, m), 2.73 (2H, t, J=6.96 Hz), 3.78-3.81 (5H, m), 3.83 (3H, s), 5.09 (2H, s), 5.28 (2H, s), 6.82 (2H, d, J=8.66 Hz), 6.92 (2H, d, J=8.66 Hz), 7.12 (1H, d, J=8.03 Hz), 7.31 (2H, d, J=8.66 Hz), 7.38 (2H, d, J=8.66 Hz), 7.47 (1H, d, J=8.03 Hz).

Step (6): Compound 1a+Compound 1b→Compound (I-1)

After a solution of compound 1a (637 mg, 0.80 mmol) in DMA (1.5 mL) was cooled to 10° C., Compound 1b (413 mg, 0.80 mmol) was added thereto and deaerated in vacuo. Sodium iodide (240 mg, 1.6 mmol) was added thereto, and then stirred at 15° C. for 6 hours. After added DMF (4.5 mL) thereto, the solution was cooled to −40° C., and added phosphorus tribromide (151 μL, 1.6 mmol), and then stirred at −40° C. for 30 minutes. The reaction mixture was slowly added to ice-cooled aqueous 5% sodium chloride. The precipitated solid was filtered, washed with water, suspended in water, and then lyophilized to yield Compound 1c as a brown solid. The obtained Compound 1c was used in the next reaction without any purification.

The whole amount of Compound 1c was dissolved in dichloromethane (10 ml). After the solution was cooled to −40° C., anisole (0.87 mL, 8.0 mmol) followed by 2 mol/L-aluminium chloride/nitromethane solution (4.0 mL, 8.0 mmol) were added thereto, and then stirred at 0° C. for 30 minutes. Diisopropyl ether and a drop of water were added to the reaction solution. After stirring, the insoluble and the supernatant were separated by decantation. The insoluble material retained in the flask was added diluted hydrochloric acid and acetonitrile, and stirred until dissolving entirely, and then diisopropyl ether was added thereto, and the aqueous layer was separated. After the organic layer was extracted with water again, all the aqueous layers were combined, and HP20-SS resin was added thereto, and then acetonitrile was removed in vacuo. The resulting mixture was purified by ODS column chromatography (water-acetonitrile). To the resulting solution containing the desired compound, aqueous 0.2 mol/L sodium hydroxide solution was added until pH=6.0, after that a small amount of dry ice was added thereto. The obtained solution was concentrated in vacuo, and then lyophilized to yield Compound I-1 as a yellow powder.

Yield: 266 mg, (43%)

¹H-NMR (D₂O) δ: 1.50 (3H, s), 1.52 (3H, s), 2.22 (4H, s), 3.55-3.71 (8H, m), 3.99-4.11 (3H, m), 4.22 (1H, d, J=14.18 Hz), 5.39 (1H, d, J=4.89 Hz), 5.88 (1H, d, J=4.89 Hz), 6.97 (1H, s), 7.00 (1H, d, J=7.78 Hz), 7.13 (1H, d, J=7.78 Hz).

### Example 2

### Synthesis of Compound (I-2)

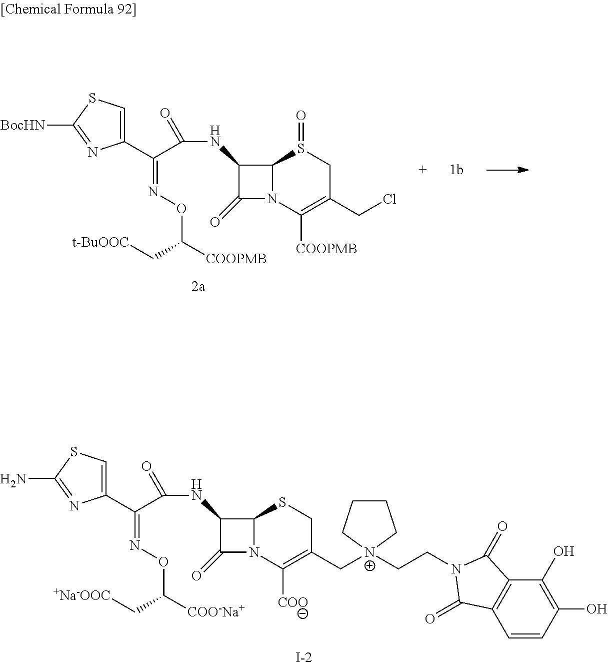

Step (1): Compound 2a+Compound 1b→Compound (I-2)

Using Compound 2a (757 mg, 0.80 mmol) and Compound 1b (413 mg, 0.80 mmol), Compound I-2 was obtained according to the same procedure as Compound I-1. Yield: 213 mg, (33%)

¹H-NMR (D₂O) δ: 2.22 (4H, s), 2.67-2.77 (2H, m), 3.52-3.70 (7H, m), 3.97-4.11 (3H, m), 4.23 (1H, d, J=14.18 Hz), 4.80 (1H, d, J=8.28 Hz), 4.98 (1H, dd, J=8.97, 4.20 Hz), 5.37 (1H, d, J=4.89 Hz), 5.86 (1H, d, J=4.89 Hz), 7.00 (1H, d, J=7.78 Hz), 7.02 (1H, s), 7.13 (1H, d, J=7.78 Hz).

MS (m+1)=774.01

### Example 3

### Synthesis of Compound (I-3)

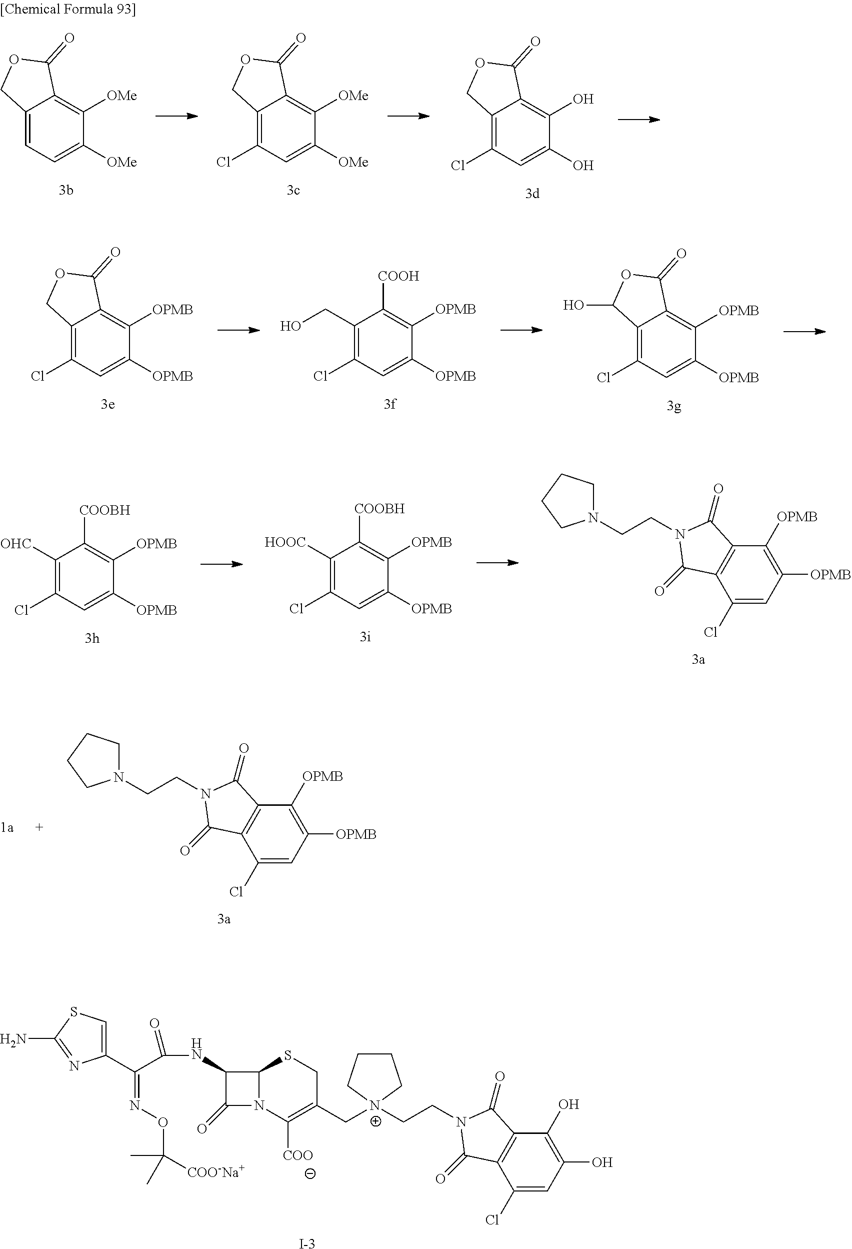

Step (1): Compound 3b→Compound 3c

A solution of Compound 3b (6.31 g, 32.5 mmol) in acetonitrile (60 mL) was added N-chlorosuccinimide (4.77 g, 35.7 mmol). After stirred at 60 degree for an hour, the insoluble material was filtered and dried to obtain Compound 3c. Yield: 6.15 g, (83%)

¹H-NMR (CDCl₃) δ: 3.91 (3H, s), 4.09 (3H, s), 5.15 (2H, s), 7.16 (1H, s).

Step (2): Compound 3c→Compound 3d

A solution of Compound 3c (6.83 g, 30 mmol) in dichloromethane (60 mL) was cooled with ice bath, and then boron tribromide (9.43 mL, 100 mmol) was added drop-wise thereto. The reaction solution was stirred at room temperature for 2 hours, after that added into ice carefully. Dichloromethane was removed in vacuo, the precipitated solid was filtered and dried to obtain Compound 3d. Yield: 5.57 g, (93%) ¹H-NMR (DMSO-D₆) δ: 5.15 (2H, s), 7.10 (1H, s).

Step (3): Compound 3d→Compound 3e

Using Compound 3d (5.57 g, 27.8 mmol), Compound 3e was obtained as a colorless solid according to the same procedure as Compound 1e. Yield: 8.45 g, (69%)

¹H-NMR (CDCl₃) δ: 3.79 (3H, s), 3.83 (3H, s), 5.03 (2H, s), 5.12 (2H, s), 5.24 (2H, s), 6.81 (2H, d, J=8.54 Hz), 6.91 (2H, d, J=8.54 Hz), 7.17 (1H, s), 7.30 (2H, d, J=8.39 Hz), 7.37 (2H, d, J=8.54 Hz).

Step (4): Compound 3e→Compound 3f

Using Compound 3d (8.45 g, 19.2 mmol), Compound 3f was obtained according to the same procedure as Compound 1f. Yield: 8.08 g, (92%)

¹H-NMR (DMSO-D₆) δ: 3.74 (3H, s), 3.77 (3H, s), 4.49 (2H, s), 4.85 (2H, s), 5.15 (2H, s), 6.85 (2H, d, J=8.54 Hz), 6.97 (2H, d, J=8.54 Hz), 7.23 (2H, d, J=8.54 Hz), 7.30 (1H, s), 7.42 (2H, d, J=8.54 Hz).

Step (5): Compound 3f→Compound 3g

A solution of Compound 3f (8.23 g, 17.9 mmol) in dichloromethane (80 mL) was cooled with ice-bath, and added Dess-Martin reagent (8.37 g, 19.7 mmol). After stirred at room temperature for 30 minutes, the reaction solution was added water, and extracted with ethyl acetate. The organic layer was dried with anhydrous magnesium sulfate, after filtered, the filtrate was concentrated in vacuo. The precipitated solid by addition diisopropyl ether into the residue was filtered and dried to obtain Compound 3g. Yield: 6.38 g, (78%)

¹H-NMR (DMSO-D₆) δ: 3.73 (3H, s), 3.77 (3H, s), 5.06 (2H, s), 5.15 (2H, s), 6.85 (2H, d, J=8.54 Hz), 6.97 (2H, d, J=8.54 Hz), 7.30 (2H, d, J=8.54 Hz), 7.38 (2H, d, J=8.39 Hz), 7.58 (1H, s).

Step (6): Compound 3g→Compound 3h→Compound 3i

A solution of Compound 3g (6.38 g, 14.0 mmol) in tetrahydrofuran (30 mL) was added drop-wise a solution of diphenyldiazomethane (2.98 g, 15.4 mmol) in tetrahydrofuran (30 mL). The mixed solution was stirred at room temperature overnight, and then the reaction solution was concentrated in vacuo. The residue was added diisopropyl ether, and then the precipitated solid was filtered and dried to obtain Compound 3h.

Using Compound 3h, the desired Compound (8.72 g, 98%) was obtained according to the same procedure as Compound 1h. Yield: 8.72 g, (98%)

¹H-NMR (DMSO-D₆) δ: 3.71 (3H, s), 3.77 (3H, s), 4.72 (2H, s), 5.19 (2H, s), 6.67 (2H, d, J=8.73 Hz), 6.79 (2H, d, J=8.73 Hz), 6.91 (1H, s), 6.98 (2H, d, J=8.73 Hz), 7.26-7.47 (13H, m).

Step (7): Compound 3i→Compound 3a

Using Compound 3i (4.16 g, 6.51 mmol), Compound 3a was obtained according to the same procedure as Compound 1i. Yield: 2.36 g, (66%)

¹H-NMR (CDCl₃) δ: 1.76 (4H, s), 2.58 (4H, s), 2.73 (2H, t, J=6.82 Hz), 3.78-3.81 (5H, m), 3.84 (3H, s), 5.06 (2H, s), 5.22 (2H, s), 6.81 (2H, d, J=8.59 Hz), 6.93 (2H, d, J=8.59 Hz), 7.03 (1H, s), 7.30-7.35 (4H, m).

Step (8): Compound 1a+Compound 3a→Compound (I-3)

Using Compound 1a (637 mg, 0.80 mmol) and Compound 3a (441 mg, 0.80 mmol), Compound I-3 was obtained according to the same procedure as Compound I-1. Yield: 68 mg, (11%)

¹H-NMR (D₂O) δ: 1.50 (3H, s), 1.52 (3H, s), 2.23 (4H, s), 3.60-3.71 (8H, m), 4.01-4.06 (3H, m), 4.22 (1H, d, J=12.51 Hz), 5.39 (1H, d, J=4.58 Hz), 5.89 (1H, d, J=4.58 Hz), 6.71 (1H, s), 6.97 (1H, s).

MS (m+1)=778.11

### Example 4

### Synthesis of Compound (I-4)

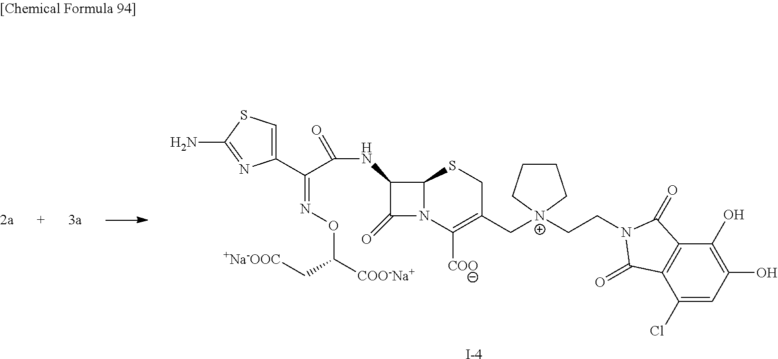

Step (1): Compound 2a+Compound 3a→Compound (I-4)

Using Compound 2a (757 mg, 0.80 mmol) and Compound 3a (441 mg, 0.80 mmol), Compound I-4 was obtained according to the same procedure as Compound I-1. Yield: 72 mg, (11%)

¹H-NMR (D₂O) δ: 2.23 (4H, s), 2.67-2.78 (2H, m), 3.54-3.75 (7H, m), 3.99-4.10 (3H, m), 4.24 (1H, d, J=14.18 Hz), 4.79 (1H, d, J=9.03 Hz), 4.97 (1H, dd, J=9.60, 3.95 Hz), 5.38 (1H, d, J=4.89 Hz), 5.86 (1H, d, J=4.89 Hz), 6.76 (1H, s), 7.02 (1H, s).

MS (m+1)=807.96

### Example 5

### Synthesis of Compound (I-5)

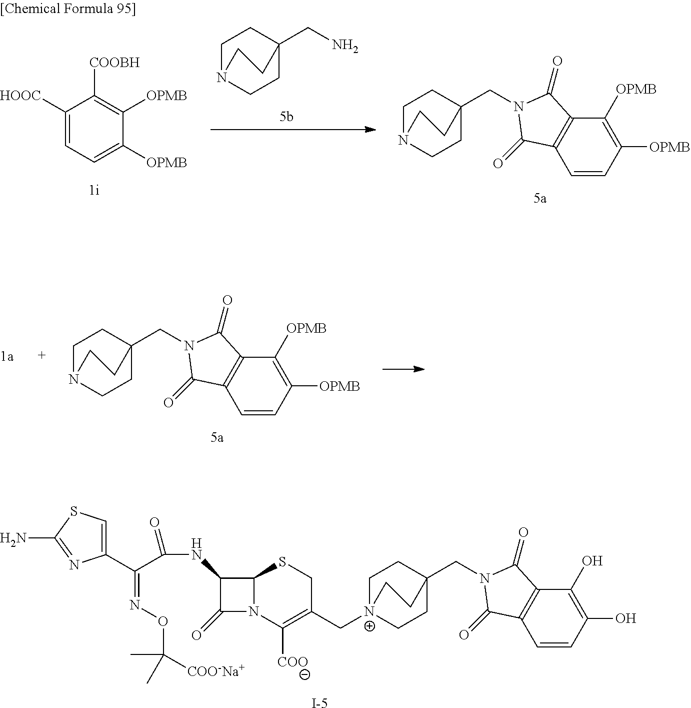

Step (1): Compound 1i→Compound 5a

Using Compound 1i (3.02 g, 5.0 mmol) and Compound 5b (771 mg, 5.5 mmol), Compound 5a was obtained according to the same procedure as Compound 1b. Yield: 2.07 g, (76%)

¹H-NMR (CDCl₃) δ: 1.41 (6H, t, J=7.65 Hz), 2.85 (6H, t, J=7.65 Hz), 3.79 (3H, s), 3.83 (3H, s), 5.11 (2H, s), 5.28 (2H, s), 6.81 (2H, d, J=8.66 Hz), 6.92 (2H, d, J=8.66 Hz), 7.14 (1H, d, J=8.03 Hz), 7.33 (2H, d, J=8.66 Hz), 7.37 (2H, d, J=8.66 Hz), 7.48 (1H, d, J=8.03 Hz).

Step (2): Compound 1a+Compound 5a→Compound (I-5)

Using Compound 1a (637 mg, 0.80 mmol) and Compound 5a (434 mg, 0.80 mmol), Compound I-5 was obtained according to the same procedure as Compound I-1. Yield: 432 mg, (68%)

¹H-NMR (D₂O) δ: 1.49 (3H, s), 1.51 (3H, s), 1.91 (6H, t, J=7.53 Hz), 3.39-3.50 (9H, m), 3.86-3.91 (2H, m), 4.58 (1H, d, J=13.93 Hz), 5.35 (1H, d, J=5.02 Hz), 5.86 (1H, d, J=5.02 Hz), 6.96 (1H, s), 7.00 (1H, d, J=7.91 Hz), 7.12 (1H, d, J=7.91 Hz).

MS (m+1)=770.05

### Example 6

### Synthesis of Compound (I-6)

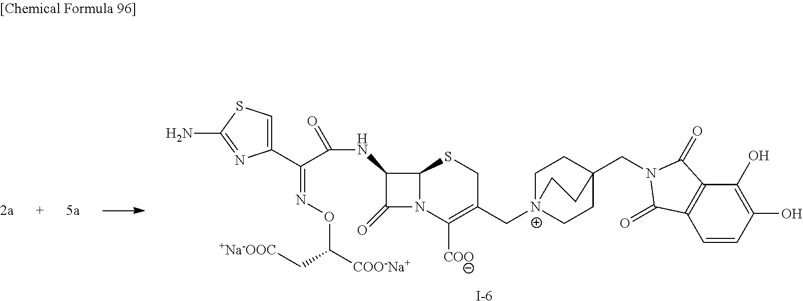

Step (1): Compound 2a+Compound 5a→Compound (I-6)

Using Compound 2a (757 mg, 0.80 mmol) and Compound 5a (434 mg, 0.80 mmol), Compound I-6 was obtained according to the same procedure as compound I-1.

Yield: 385 mg, (57%)

¹H-NMR (D₂O) δ: 1.93 (6H, t, J=7.65 Hz), 2.70-2.72 (2H, m), 3.38-3.53 (9H, m), 3.83-3.92 (2H, m), 4.57 (1H, d, J=13.93 Hz), 4.97 (1H, dd, J=8.66, 4.64 Hz), 5.32 (1H, d, J=4.89 Hz), 5.83 (1H, d, J=4.89 Hz), 7.00 (1H, s), 7.06 (1H, d, J=7.78 Hz), 7.19 (1H, d, J=7.78 Hz).

MS (m+1)=800.00

### Example 7

### Synthesis of Compound (I-7)

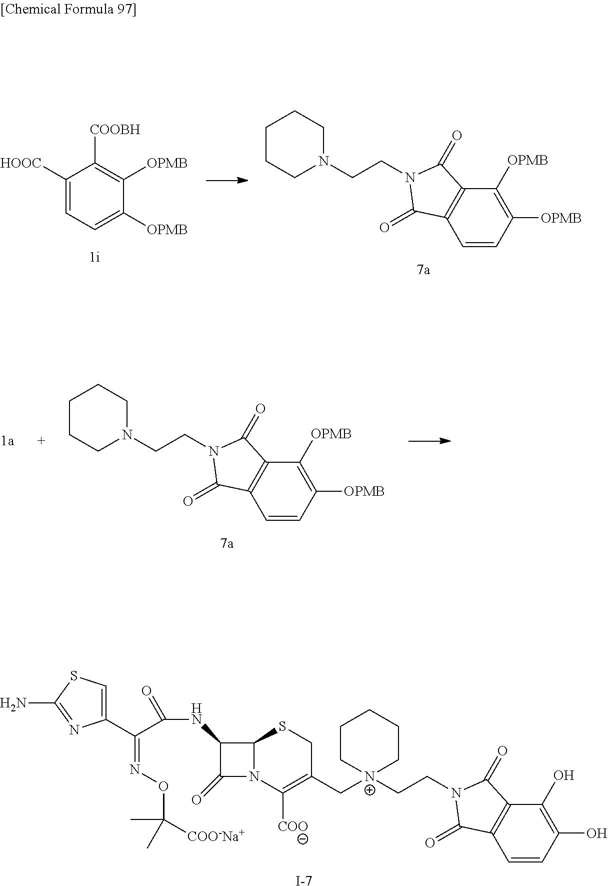

Step (1): Compound 1i→Compound 7a

Using Compound 1i (3.02 g, 5.0 mmol) and 1-(2-aminoethyl)piperidine (775 μL, 5.5 mmol), Compound 7a was obtained according to the same procedure as Compound 1b. Yield: 2.51 g, (95%)

¹H-NMR (CDCl₃) δ: 1.38-1.43 (2H, m), 1.51-1.56 (4H, m), 2.46 (4H, s), 2.57 (2H, t, J=6.96 Hz), 3.76-3.79 (5H, m), 3.83 (3H, s), 5.09 (2H, s), 5.28 (2H, s), 6.82 (2H, d, J=8.53 Hz), 6.92 (2H, d, J=8.53 Hz), 7.11 (1H, d, J=8.16 Hz), 7.30-7.39 (4H, m), 7.47 (1H, d, J=8.16 Hz).

Step (2): Compound 1a+Compound 7a→Compound (I-7)

Using Compound 1a (637 mg, 0.80 mmol) and Compound 7a (531 mg, 0.80 mmol), Compound I-7 was obtained according to the same procedure as Compound I-1. Yield: 155 mg, (25%)

¹H-NMR (D₂O) δ: 1.50 (3H, s), 1.52 (3H, s), 1.78-1.94 (5H, m), 3.29-3.77 (8H, m), 3.97-4.09 (4H, m), 4.23 (1H, d, J=14.05 Hz), 5.40 (1H, d, J=5.02 Hz), 5.89 (1H, d, J=5.02 Hz), 6.99 (1H, s), 7.02 (1H, d, J=7.91 Hz), 7.17 (1H, d, J=7.91 Hz).

MS (m+1)=758.03

### Example 8

### Synthesis of Compound (I-8)

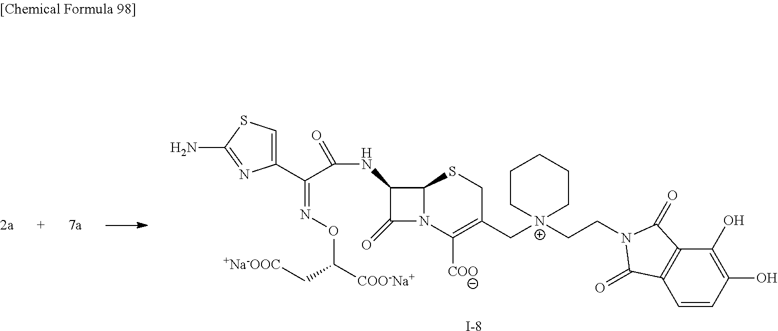

Step (1): Compound 2a+Compound 7a→Compound (I-8)

Using Compound 2a (757 mg, 0.80 mmol) and Compound 7a (531 mg, 0.80 mmol), a compound I-8 was obtained according to the same procedure as Compound I-1. Yield: 165 mg, (25%)

¹H-NMR (D₂O) δ: 1.57 (1H, s), 1.77-1.95 (5H, m), 2.71-2.74 (2H, m), 3.30-3.75 (7H, m), 3.97-4.11 (3H, m), 4.25 (1H, d, J=14.05 Hz), 4.88 (1H, d, J=14.05 Hz), 4.98 (1H, dd, J=8.91, 4.39 Hz), 5.37 (1H, d, J=4.89 Hz), 5.85 (1H, d, J=4.89 Hz), 7.03 (1H, s), 7.05 (1H, d, J=7.78 Hz), 7.20 (1H, d, J=7.78 Hz).

MS (m+1)=788.02

### Example 9

### Synthesis of Compound (I-9)

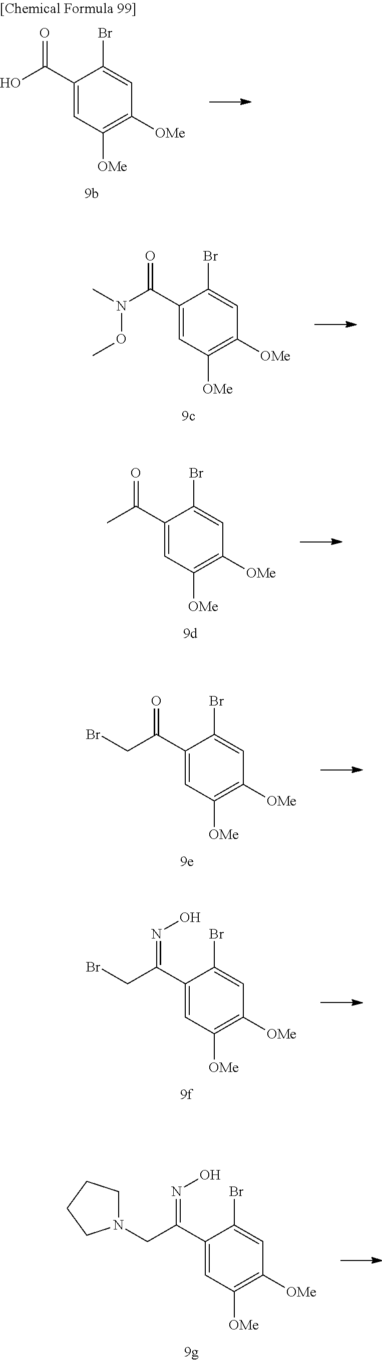 [SMILES: COc1cc(Br)c(C(=O)O)cc1OC ; COc1cc(Br)c(C(=O)N(C)OC)cc1OC ; COc1cc(Br)c(C(C)=O)cc1OC ; COc1cc(Br)c(C(=O)CBr)cc1OC ; COc1cc(Br)c(/C(CBr)=N\O)cc1OC ; COc1cc(Br)c(/C(CN2CCCC2)=N\O)cc1OC]

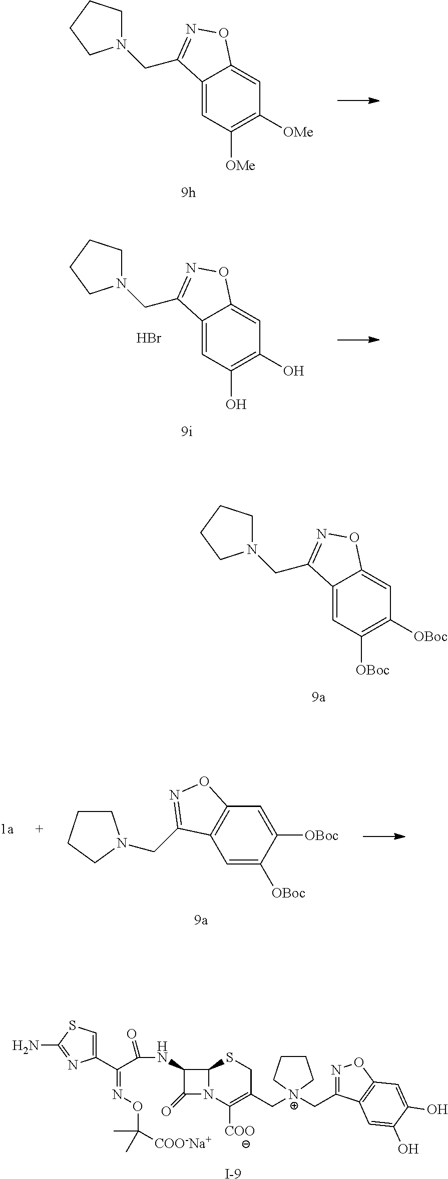

Step (1): Compound 9b→Compound 9c→Compound 9d

A solution of Compound 9b (24.4 g, 93 mmol) in dichloromethane (120 mL) was cooled with ice-bath, N,O-dimethylhydroxylamine hydrochloride salt (16.4 g, 168 mmol) and 1-ethyl-3-(3-dimethylaminopropyl)carbodiimido hydrochloride salt (32.2 g, 168 mmol) was added thereto. After stirred at room temperature for four and a half hours, the reaction solution was added water. The reaction solution was extracted with dichloromethane, and then the organic layer was dried with anhydrous magnesium sulfate and filtered. The filtrate was concentrated in vacuo to obtain Compound 9c as orange oil. Compound 9c was used in the next reaction without any purification.

The obtained above Compound 9c was dissolved in tetrahydrofuran (500 mL), and cooled with ice-bath. This solution was added 1 mol/L bromomethyl magnesium/tetrahydrofuran solution (186 mL, 186 mmol). After stirred at room temperature for 5 hours, ammonium chloride solution was added thereto, and that the reaction mixture was extracted with ethyl acetate. The organic layer was dried with anhydrous magnesium sulfate and filtered, after that the filtrate was concentrated in vacuo. The precipitated solid by addition diisopropyl ether into the residue was filtered and dried to obtain Compound 9d. Yield: 23.5 g (97%)

¹H-NMR (CDCl₃) δ: 2.68 (3H, s), 3.90 (3H, s), 3.92 (3H, s), 7.05 (1H, s), 7.15 (1H, s)

Step (2): Compound 9d→Compound 9e

A solution of copper bromide (7.97 g, 35.7 mmol) in ethyl acetate (20 mL) was refluxed, and added a solution of Compound 9d (5.0 g, 19.3 mmol) in chloroform (20 mL). After refluxed for 2 and a half hours, the insoluble material was filtered out, the filtrate was concentrated in vacuo. The residue was purified by silica gel chromatography(ethyl acetate/n-hexane) to obtain Compound 9e. Yield: 4.61 g (71%)

¹H-NMR (CDCl₃) δ: 3.90 (3H, s), 3.93 (3H, s), 4.59 (2H, s), 7.06 (1H, s), 7.13 (1H, s)

Step (3): Compound 9e→Compound 9f→Compound 9g

A solution of Compound 9e (4.61 g, 13.6 mmol) in methanol (180 mL) and water (30 mL) was added hydroxylamine hydrochloride salt (7.58 g, 109 mmol). After stirred at room temperature overnight, the reaction was quenched by addition hydrochloric acid. The reaction solution was extracted with ethyl acetate, the organic layer was washed with water followed by saturated brine, and then dried with anhydrous magnesium sulfate. After filtered, the filtrate was concentrated to dryness to obtain Compound 9f as yellow oil. Compound 9f was used in the next reaction without any purification.

The obtained above Compound 9f was dissolved in tetrahydrofuran (50 mL), added pyrrolidine (3.38 mL, 40.9 mmol), and then stirred at room temperature for an hour. After added water, the reaction solution was extracted with ethyl acetate. The organic layer was washed with water, followed by saturated brine, and dried with anhydrous magnesium sulfate. After filtered, the filtrate was concentrated and dried to obtain Compound 9g as a yellow solid. Yield: 4.63 g (99%)

¹H-NMR (CDCl₃) δ: 1.77-1.80 (4H, m), 2.64 (4H, br s), 3.49 (2H, s), 3.86 (3H, s), 3.88 (3H, s), 6.68 (1H, s), 7.06 (1H, s).

Step (4): Compound 9g→Compound 9h

A solution of Compound 9g (4.29 g, 12.5 mmol) in 1,4-dioxane (300 mL) was added sodium tert-butoxide (1.80 g, 18.8 mmol). The reaction solution was deaerated under reduced pressure, after that palladium acetate (421 mg, 1.88 mmol) and 1,3-bis(diphenylphosphino)propane (1.19 mg, 2.88 mmol). The reaction solution was stirred at 80 degree for 4 hours, after that it was added water and extracted with ethyl acetate. The organic layer was washed with saturated brine, dried with anhydrous magnesium sulfate and filtered. After the filtrate was concentrated, the residue was purified by silica gel chromatography (ethyl acetate/n-hexane) to obtain Compound 9h as yellow oil. Yield: 1.82 g (56%)

¹H-NMR (CDCl₃) δ: 1.79-1.83 (4H, m), 2.60-2.63 (4H, m), 3.94 (3H, s), 3.97 (3H, s), 3.98 (2H, s), 7.02 (1H, s), 7.18 (1H, s).

Step (5): Compound 9h→Compound 9i→Compound 9a

A solution of Compound 9h (1.82 g, 6.94 mmol) in dichloromethane (20 mL) was cooled with ice-bath, and added drop-wise boron tribromide (1.97 mL, 20.8 mmol). The reaction solution was stirred for one and a half hours, and then added methanol thereto carefully. The solvent was removed in vacuo, the residue was dried to obtain the solid containing Compound 9i.

The synthesized above Compound 9i was suspended in dichloromethane (20 mL), and cooled with ice-bath. This solution was added triethylamine (1.44 mL, 10.4 mmol), N, N-dimethylaminopyridine (42 mg, 0.35 mmol), ditert-butyl dicarbonate (4.83 mL, 20.8 mmol). The reaction solution was stirred at room temperature overnight, after that added water and extracted with ethyl acetate. The organic layer was washed with water followed by saturated brine, and dried with anhydrous sodium sulfate. After filtered, the obtained residue by concentration of the filtrate was purified with silica gel chromatography (ethyl acetate including 3% triethylamine/n-hexane) to obtain Compound 9a as a yellow solid. Yield: 602 mg (20%)

¹H-NMR (CDCl₃) δ: 1.57 (18H, s), 1.78-1.81 (4H, m), 2.58-2.61 (4H, m), 4.00 (2H, s), 7.51 (1H, s), 7.78 (1H, s).

Step (6): Compound 1a+Compound 9a→Compound (I-9)

Using Compound 1a (796 mg, 1.0 mmol) and Compound 9a (434 mg, 1.0 mmol), Compound I-9 was synthesized according to the same procedure as Compound I-1.

Yield: 476 mg (66%)

¹H-NMR (D₂O) δ: 1.49 (3H, s), 1.50 (3H, s), 2.23 (4H, s), 3.47 (1H, d, J=16.94 Hz), 3.70-3.90 (5H, m), 4.37 (1H, d, J=13.93 Hz), 4.86 (1H, d, J=13.93 Hz), 4.93 (2H, d, J=7.65 Hz), 5.32 (1H, d, J=5.02 Hz), 5.85 (1H, d, J=5.02 Hz), 6.95 (1H, s), 7.12 (1H, s), 7.17 (1H, s).

MS (m+1)=702.27

Elemental Analysis: C₂₉H₃₀N₇O₁₀S₂Na.5.4H₂O

Calculated: C, 42.43; H, 5.01; N, 11.94; S, 7.81; Na, 2.80(%).

Found: C, 42.36; H, 4.98; N, 12.19; S, 7.71; Na, 2.88(%).

### Example 10

### Synthesis of Compound (I-10)

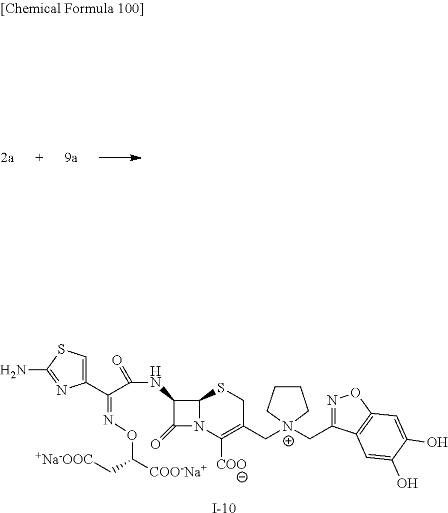

Step (1): Compound 2a+Compound 9a→Compound (I-10)

Using Compound 2a (946 mg, 1.0 mmol) and Compound 9a (434 mg, 1.0 mmol), Compound I-10 was synthesized according to the same procedure as Compound I-1.

Yield: 451 mg, (58%)

¹H-NMR (D₂O) δ: 2.17-2.30 (4H, br m), 2.69-2.72 (2H, m), 3.49 (1H, d, J=16.94 Hz), 3.68-3.89 (5H, m), 4.37 (1H, d, J=13.80 Hz), 4.86-4.99 (4H, m), 5.30 (1H, d, J=4.89 Hz), 5.81 (1H, d, J=4.89 Hz), 7.00 (1H, s), 7.16 (1H, s), 7.20 (1H, s).

MS (m+1)=732.22

Elemental Analysis: C₂₉H₂₇N₇O₁₂S₂Na₂.7.4H₂O

Calculated: C, 38.32; H, 4.64; N, 10.79; S, 7.06; Na, 5.06(%).

Found: C, 38.26; H, 4.61; N, 11.08; S, 6.89; Na, 4.94(%).

### Example 11

### Synthesis of Compound (I-11)

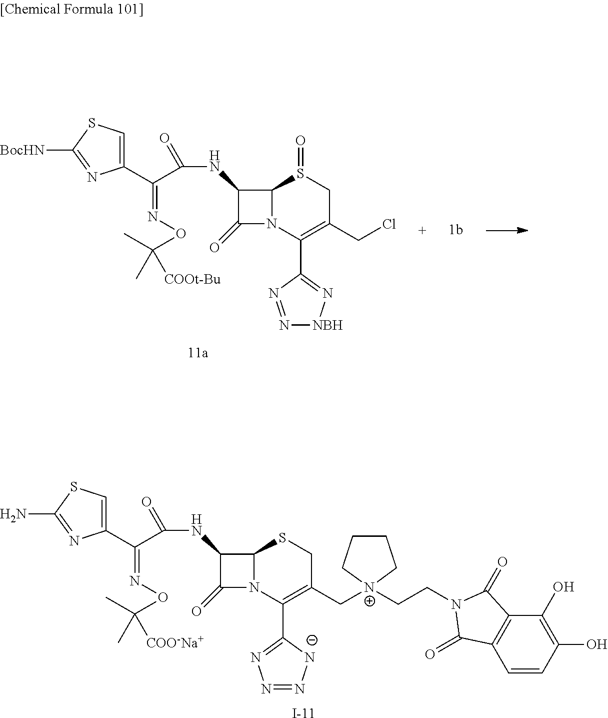

Step (1): Compound 11a+Compound 1b→Compound (I-11)

Using Compound 11a (693 mg, 0.80 mmol)(the synthesis method was described in WO2012/147773) and Compound 1b (413 mg, 0.80 mmol), Compound I-11 was synthesized according to the same procedure as Compound I-1. Yield: 238 mg, (38%)

¹H-NMR (D₂O) δ: 1.21 (1H, s), 1.49 (3H, s), 1.52 (3H, s), 2.13-1.90 (3H, m), 3.40-3.86 (10H, m), 4.16 (1H, d, J=17.19 Hz), 4.21 (1H, d, J=14.81 Hz), 5.59 (1H, d, J=5.02 Hz), 5.93 (1H, d, J=5.02 Hz), 6.92 (1H, s), 6.94 (1H, d, J=7.91 Hz), 7.04 (1H, d, J=7.91 Hz).

MS (m+1)=768.04

Elemental Analysis: C₃₁H₃₂N₁₁O₉S₂Na.5.3H₂O

Calculated: C, 42.06; H, 4.85; N, 17.40; Na, 2.60; S, 7.24(%).

Found: C, 42.03; H, 4.80; N, 17.57; Na, 2.68; S, 7.22(%).

### Example 12

### Synthesis of Compound (I-12)

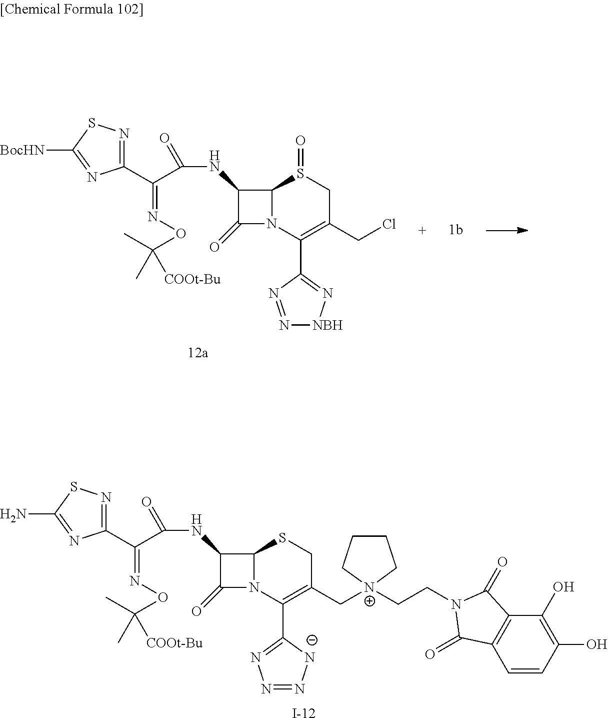

Step (1): Compound 12a+Compound 1b→Compound (I-12)

Using Compound 12a (694 mg, 0.80 mmol) (the synthesis method was described in WO2012/147773) and Compound 1b (413 mg, 0.80 mmol), Compound I-12 was synthesized according to the same procedure as Compound I-1. Yield: 100 mg, (16%)

¹H-NMR (D₂O) δ: 1.19 (1H, s), 1.54 (3H, s), 1.56 (3H, s), 1.93-2.11 (3H, m), 3.40-3.87 (10H, m), 4.17 (1H, d, J=17.19 Hz), 4.22 (1H, d, J=14.56 Hz), 5.61 (1H, d, J=5.14 Hz), 5.97 (1H, d, J=5.14 Hz), 6.99 (1H, d, J=7.78 Hz), 7.12 (1H, d, J=7.78 Hz).

MS (m+1)=769.04

Elemental Analysis: C₃₀H₃₁N₁₂O₉S₂Na.6.5H₂O.0.1 NaHCO₃

Calculated: C, 39.46; H, 4.85; N, 18.34; Na, 2.76; S, 7.00(%).

Found: C, 39.41; H, 4.83; N, 18.36; Na, 2.86; S, 6.90(%).

### Example 13

### Synthesis of Compound (I-13)

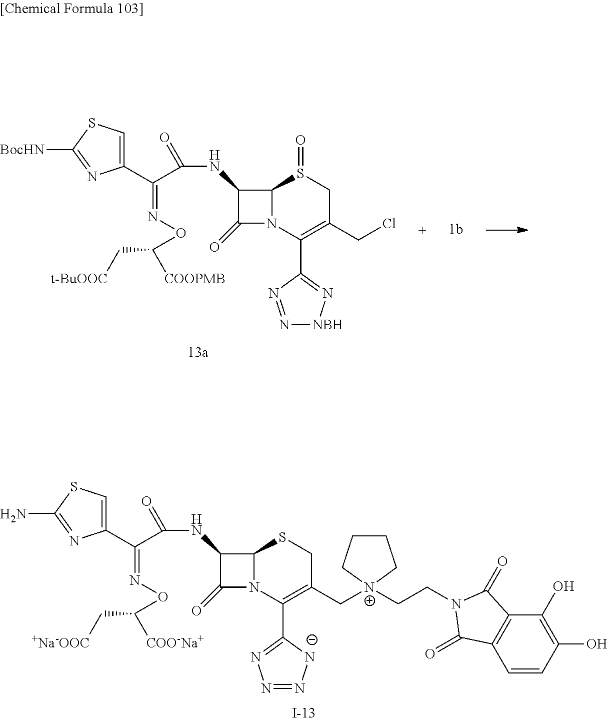

Step (1): Compound 13a+Compound 1b→a Compound (I-13)

Using Compound 13a (813 mg, 0.80 mmol) and Compound 1b (413 mg, 0.80 mmol), Compound I-13 was synthesized according to the same procedure as Compound I-1. Yield: 233 mg, (35%)

¹H-NMR (D₂O) δ: 1.29 (1H, s), 1.93-2.11 (3H, m), 2.71-2.74 (2H, m), 3.34-3.91 (10H, m), 4.13 (1H, d, J=17.07 Hz), 4.25 (1H, d, J=14.43 Hz), 4.98 (1H, dd, J=9.35, 3.95 Hz), 5.58 (1H, d, J=5.02 Hz), 5.89 (1H, d, J=5.02 Hz), 6.99 (1H, s), 7.00 (1H, d, J=8.03 Hz), 7.12 (1H, d, J=8.03 Hz).

MS (m+1)=708.02

Elemental Analysis: C₃₁H₂₉N₁₁O₁₁S₂Na₂.6.6H₂O

Calculated: C, 38.76; H, 4.43; N, 16.04; Na, 4.79; S, 6.68(%).

Found: C, 38.77; H, 4.48; N, 16.08; Na, 4.68; S, 6.54(%).

### Example 14

### Synthesis of Compound (I-14)

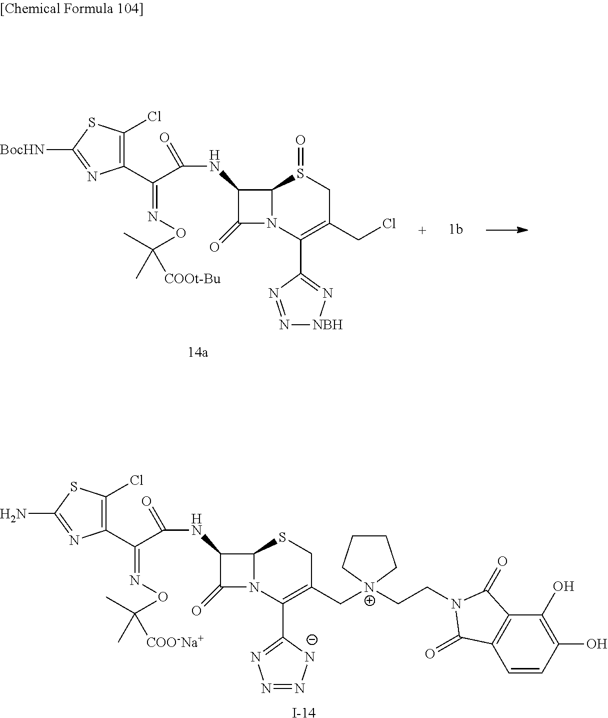

Step (1): Compound 14a+Compound 1b→Compound (I-14)

Using Compound 14a (721 mg, 0.80 mmol) and Compound 1b (413 mg, 0.80 mmol), Compound I-14 was synthesized according to the same procedure as Compound I-1. Yield: 198 mg, (30%)

¹H-NMR (D₂O) δ: 1.22 (1H, s), 1.51 (3H, s), 1.53 (3H, s), 1.94-2.10 (3H, m), 3.40-3.87 (10H, m), 4.16 (1H, d, J=17.19 Hz), 4.22 (1H, d, J=14.31 Hz), 5.60 (1H, d, J=5.14 Hz), 5.95 (1H, d, J=5.14 Hz), 6.98 (1H, d, J=7.91 Hz), 7.10 (1H, d, J=7.91 Hz).

MS (m+1)=801.98

Elemental Analysis: C₃₁H₃₁ClN₁₁O₉S₂Na.5.2H₂O

Calculated: C, 40.56; H, 4.55; Cl, 3.86; N, 16.79; Na, 2.50; S, 6.99(%).

Found: C, 40.44; H, 4.58; Cl, 3.89; N, 16.99; Na, 2.50; S, 6.95(%).

### Example 15

### Synthesis of Compound (I-15)

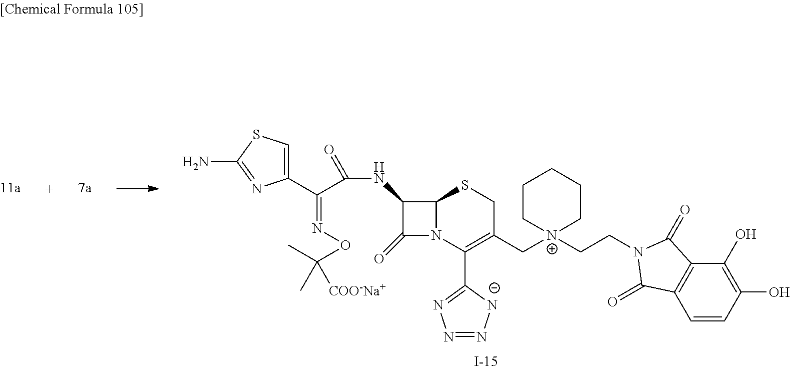

Step (1): Compound 11a+Compound 7a→Compound (I-15)

Using Compound 11a (866 mg, 1.0 mmol) and Compound 7a (531 mg, 1.0 mmol), Compound I-15 was synthesized according to the same procedure as Compound I-1.

Yield: 258 mg, (26%)

¹H-NMR (D₂O) δ: 1.33-1.45 (2H, m), 1.49 (3H, s), 1.52 (3H, s), 1.63-1.92 (4H, m), 3.00-3.11 (2H, m), 3.27 (1H, t, J=9.72 Hz), 3.39-3.57 (3H, m), 3.67-3.83 (3H, m), 4.15 (1H, d, J=16.94 Hz), 4.25 (1H, d, J=14.31 Hz), 4.95 (1H, d, J=14.31 Hz), 5.60 (1H, d, J=5.14 Hz), 5.93 (1H, d, J=5.14 Hz), 6.89 (1H, d, J=7.53 Hz), 6.97 (1H, s), 7.03 (1H, dd, J=7.65, 2.89 Hz).

MS (m+1)=782.04

### Example 16

### Synthesis of Compound (I-16)

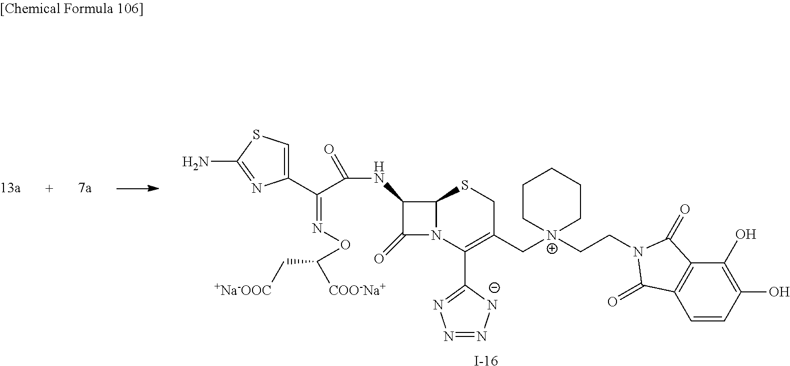

Step (1): Compound 13a+Compound 7a→Compound (I-15)

Using Compound 13a (1.02 g, 1.0 mmol) and Compound 7a (531 mg, 1. mmol), Compound I-16 was synthesized according to the same procedure as Compound I-1.

Yield: 273 mg, (25%)

¹H-NMR (D₂O) δ: 1.47 (2H, s), 1.63-1.82 (4H, m), 2.69-2.71 (2H, m), 3.06-3.11 (2H, m), 3.25-3.30 (1H, m), 3.35-3.50 (2H, m), 3.53-3.60 (1H, m), 3.65-3.74 (2H, m), 3.79 (1H, d, J=17.07 Hz), 4.12 (1H, d, J=16.94 Hz), 4.27 (1H, d, J=14.31 Hz), 4.94-4.99 (2H, m), 5.58 (1H, d, J=4.89 Hz), 5.89 (1H, d, J=4.89 Hz), 6.86 (1H, d, J=7.65 Hz), 7.01 (1H, s), 7.03 (1H, d, J=7.65 Hz).

MS (m+1)=812.03

### Example 17

### Synthesis of Compound (I-17)

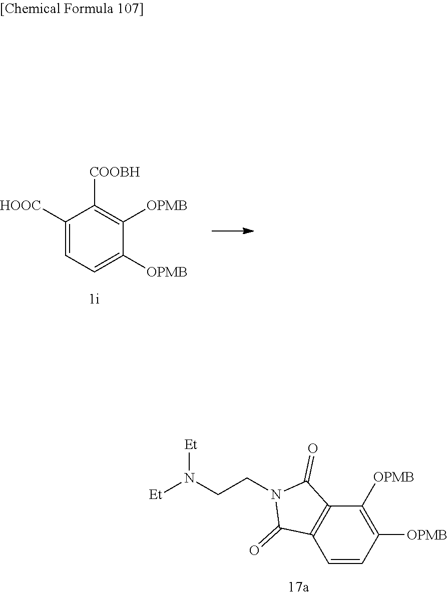

Step (1): Compound 1i→Compound 17a

Using Compound 1i (3.63 g, 6.0 mmol) and N,N-diethylethlenediamine (1.01 mL, 7.2 mmol), Compound 17a was synthesized according to the same procedure as Compound 1b. Yield: 1.98 g, (64%)

¹H-NMR (CDCl₃) δ: 7.47 (1H, d, J=8.03 Hz), 7.38 (2H, d, J=8.53 Hz), 7.31 (2H, d, J=8.53 Hz), 7.11 (1H, d, J=8.03 Hz), 6.92 (2H, d, J=8.50 Hz), 6.82 (2H, d, J=8.53 Hz), 5.28 (2H, s), 5.08 (2H, s), 3.83 (3H, s), 3.79 (3H, s), 3.73 (2H, t, J=7.10 Hz), 2.69 (2H, t, J=7.09 Hz), 2.58 (4H, q, J=7.11 Hz), 1.01 (6H, t, J=7.09 Hz).

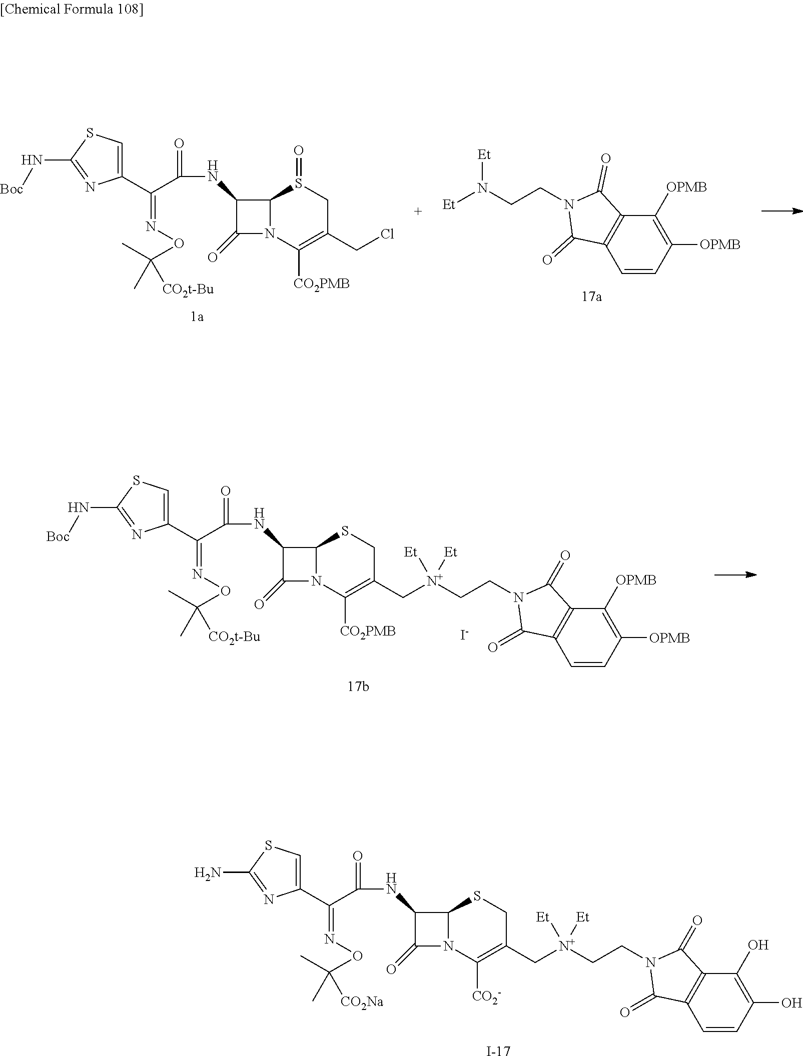

Step (2): Compound 1a+Compound 17a→Compound 17b→Compound I-17

Using Compound 1a (796 mg, 1.00 mmol) and Compound 17a (545 mg, 1.05 mmol), Compound I-17 was synthesized according to the same procedure as Compound I-1. Yield: 169.9 mg, (18%)

¹H-NMR (D₂O) δ: 7.21 (1H, d, J=7.8 Hz), 7.05 (1H, d, J=7.8 Hz), 6.99 (1H, s), 5.89 (1H, d, J=5.0 Hz), 5.37 (1H, d, J=5.0 Hz), 4.14-3.97 (4H, m), 3.59 (1H, d, J=16.8 Hz), 3.52-3.42 (6H, m), 1.52 (3H, s), 1.50 (3H, s), 1.45-1.40 (6H, m).

Elemental Analysis: C31H34N7O11S2Na(H2O)6.2

Calculated: C, 42.34; H, 5.32; N, 11.15; S, 7.29; Na, 2.61(%).

Found: C, 42.27; H, 5.21; N, 11.28; S, 7.25; Na, 2.45(%).

### Example 18

### Synthesis of Compound (I-18)

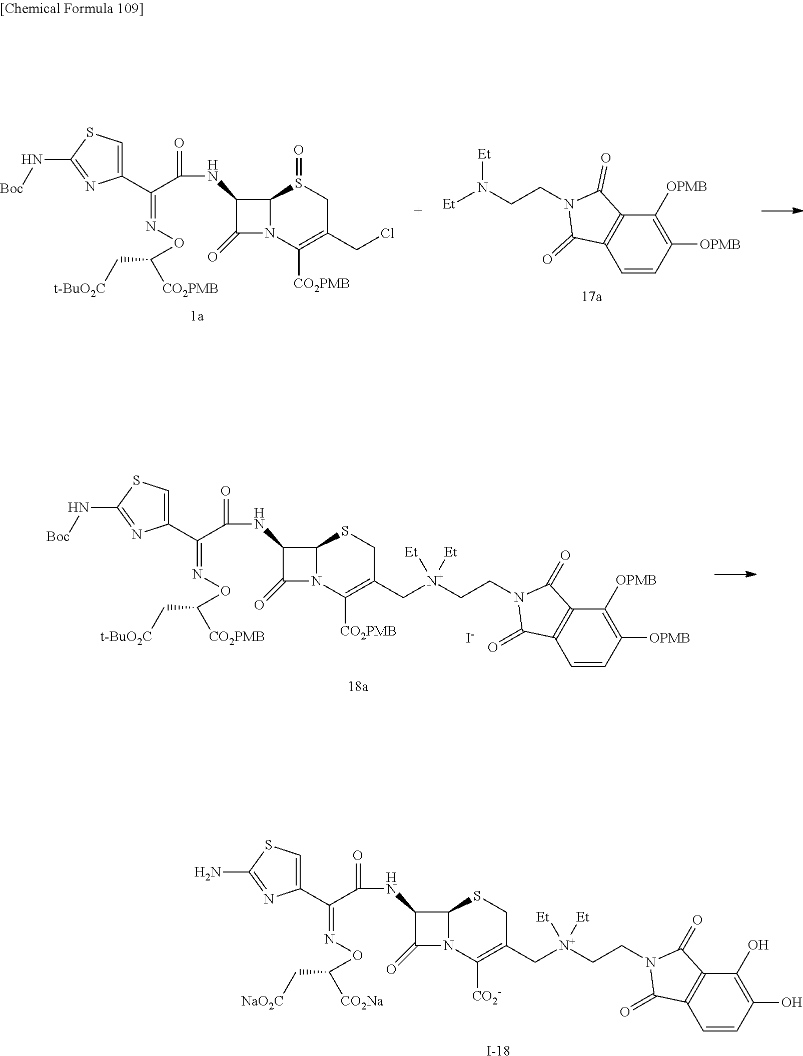

Step (1): Compound 2a+Compound 17a→Compound 18a→Compound I-18

Using Compound 2a (946 mg, 1.00 mmol) and Compound 17a (545 mg, 1.05 mmol), Compound I-18 was synthesized according to the same procedure as Compound I-1. Yield: 157.6 mg, (16%)

¹H-NMR (D₂O) δ: 7.24 (1H, d, J=7.8 Hz), 7.07 (1H, d, J=7.8 Hz), 7.02 (1H, s), 5.85 (1H, d, J=4.9 Hz), 5.35 (1H, d, J=4.9 Hz), 4.14 (1H, d, J=14.8 Hz), 4.07-4.05 (2H, m), 3.95 (1H, d, J=16.3 Hz), 3.57 (1H, d, J=16.9 Hz), 3.51-3.43 (6H, m), 2.76-2.70 (2H, m), 1.43 (6H, t, J=6.8 Hz).

Elemental Analysis: C31H31.3N7O13S2Na1.7 (H2O)7

Calculated: C, 39.64; H, 4.86; N, 10.44; S, 6.83; Na, 4.16(%).

Found: C, 39.59; H, 4.78; N, 10.57; S, 6.78; Na, 4.15(%).

### Example 19

### Synthesis of Compound (I-19)

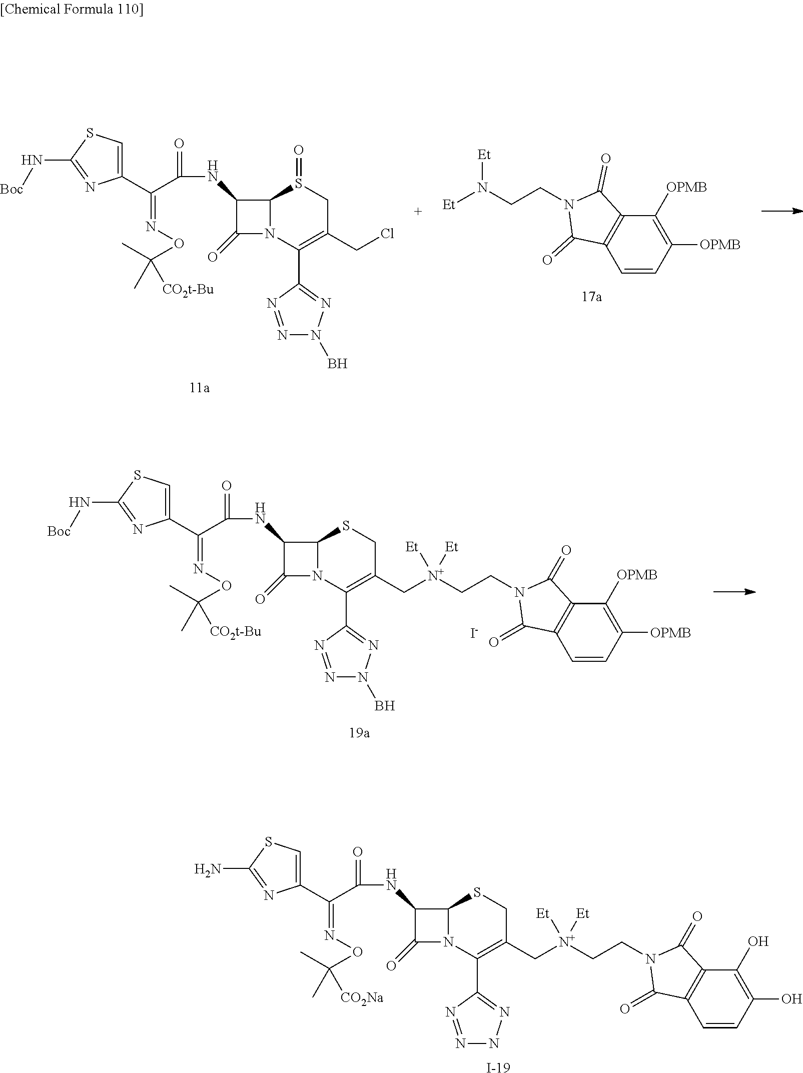

Step (1): Compound 11a+Compound 17a→Compound 19a→Compound I-19

Using Compound 11a (866 mg, 1.00 mmol) and Compound 17a (545 mg, 1.05 mmol), Compound I-19 was synthesized according to the same procedure as Compound I-1. Yield: 269.2 mg, (29%)

¹H-NMR (D₂O) δ: 7.07-6.93 (3H, m), 5.93 (1H, br s), 5.56 (1H, br s), 4.14 (2H, d, J=14.1 Hz), 3.92-3.79 (3H, m), 3.41-3.14 (6H, m), 1.51 (3H, s), 1.48 (3H, s), 1.08 (6H, br s).

Elemental Analysis: C31H34N11O9S2Na(H2O)5.7

Calculated: C, 41.63; H, 5.12; N, 17.23; S, 7.17; Na, 2.57(%).

Found: C, 41.61; H, 5.05; N, 17.30; S, 7.05; Na, 2.71(%).

### Example 20

### Synthesis of Compound (I-20)

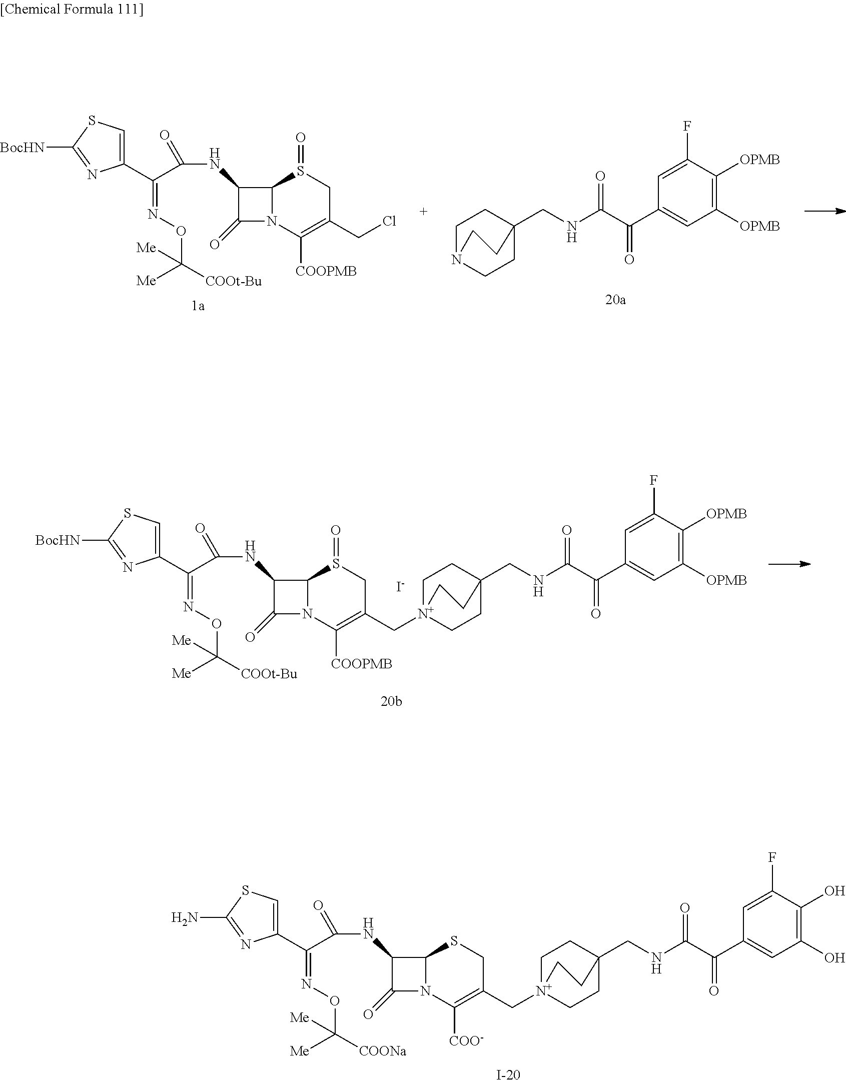

A solution of Compound 1a (884 mg, 1.11 mmol) in DMF (2 ml) was added sodium iodide (333 mg, 2.22 mmol) at 15° C., and stirred for 30 minutes. A pre-cooled to 0° C. solution of Compound 20a (752 mg, 1.11 mmol) in DMF (1 ml) was added into the reaction solution. The reaction solution was stirred at 0° C. for 3 hours, after that stood at −20° C. for 3 days. The reaction solution was cooled to −50° C. and added phosphorus tribromide (209 μl, 2.2 mmol). After the reaction solution was stirred at −40° C. for 40 minutes, the reaction solution was added into a mixed solution of water and ethyl acetate. The organic layer was washed with water and brine. The organic layer was dried with anhydrous magnesium sulfate, filtered, and then the filtrate was concentrated in vacuo. The residue was diluted with dichloromethane (24 ml) and added anisole (1.46 ml, 13.3 mmol). The reaction solution was cooled to −30° C., and added a solution of aluminum chloride in nitromethane (2 mol/L, 6.66 ml, 13.3 mmol). The reaction solution was stirred at −30° C. for 40 minutes, after that diisopropyl ether (30 ml) and 2 mol/L-HCl was added thereto. The reaction solution was stirred at 0° C. for 30 minutes, and then the solution was removed by decantation, after that the residue was dissolved with diluted hydrochloric acid and acetnitrile. After added HP20-SS into the solution and concentrated, the suspension was subjected to ODS column connected with HP20-SS column, eluting with 20 mmol/L sulfuric acid aqueous solution and acetonitrile. The fractions containing the desired compound were collected and added HP20-SS thereto. The obtained suspension was concentrated, after that the residue was subjected to HP20-SS column, eluting with water and acetonitrile. The fractions containing the desired compound was collected, and neutralized with 0.2 mol/L sodium hydrate solution, adjusted pH=5.7. The solution was concentrated in vacuo, and then lyophilized to obtain Compound I-20 (677 mg, 75%) as a pale yellow powder.

H-NMR (d₆-DMSO) δ: 1.50 (3H, s), 1.52 (3H, s), 1.94 (6H, m), 3.36-3.53 (9H, m), 3.89 (2H, m), 4.61 (1H, d, J=14.0 Hz), 5.36 (1H, d, J=5.2 Hz), 5.87 (1H, d, J=4.8 Hz), 6.99 (1H, s), 7.34 (1H, s), 7.43 (1H, d, J=11.2 Hz).

### Example 21

### Synthesis of Compound (I-21)

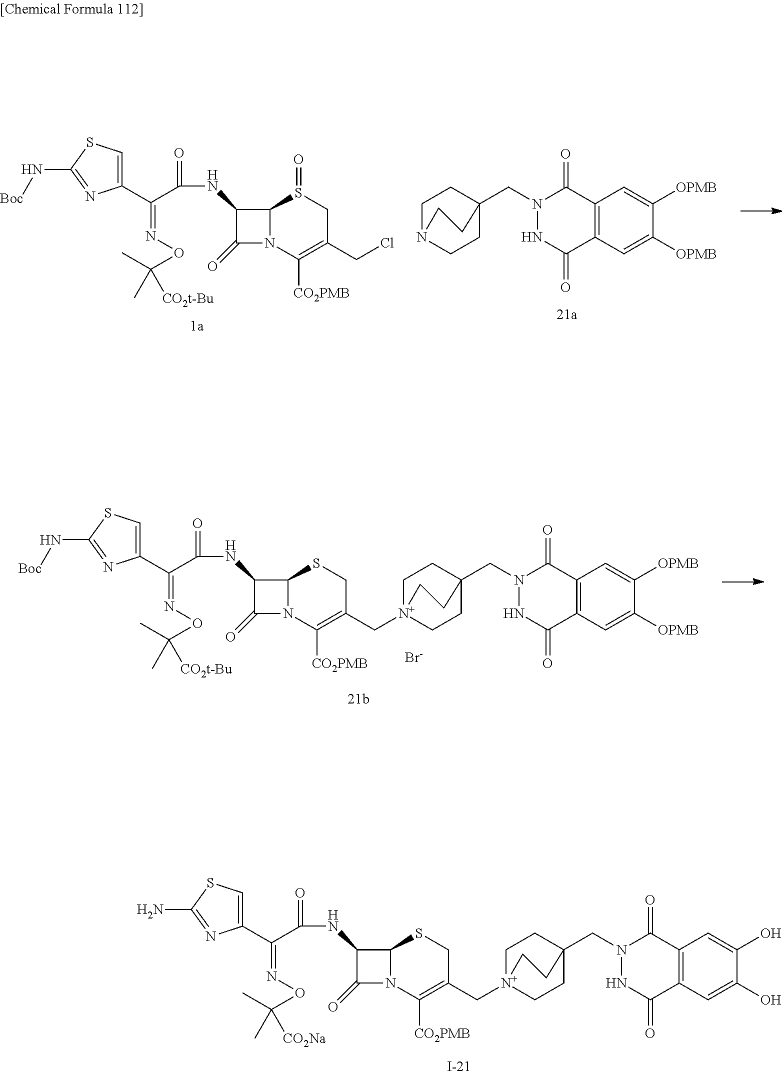

Using Compound 1a (796 mg, 1.0 mmol) and Compound 21a (558 mg, 1.0 mmol), Compound 1-21 was synthesized according to the same procedure as Compound 1-20. Yield: 202.7 mg, (21%)

¹H-NMR (D₂O) δ: 7.46 (1H, s), 7.32 (1H, s), 6.96 (1H, s), 5.86 (1H, d, J=4.9 Hz), 5.35 (1H, d, J=4.9 Hz), 4.56 (1H, d, J=13.8 Hz), 4.00 (2H, br s), 3.89-3.85 (2H, m), 3.54-3.37 (7H, m), 2.00 (6H, br s), 1.50 (3H, s), 1.49 (3H, s).

Elemental Analysis: C33H34.9N8O11S2Na1.1 (H2O)6.8

Calculated: C, 42.55; H, 5.25; N, 12.03; S, 6.88; Na, 2.71(%).

Found: C, 42.39; H, 5.18; N, 12.23; S, 6.89; Na, 2.78(%).

### Example 22

### Synthesis of Compound (I-22)

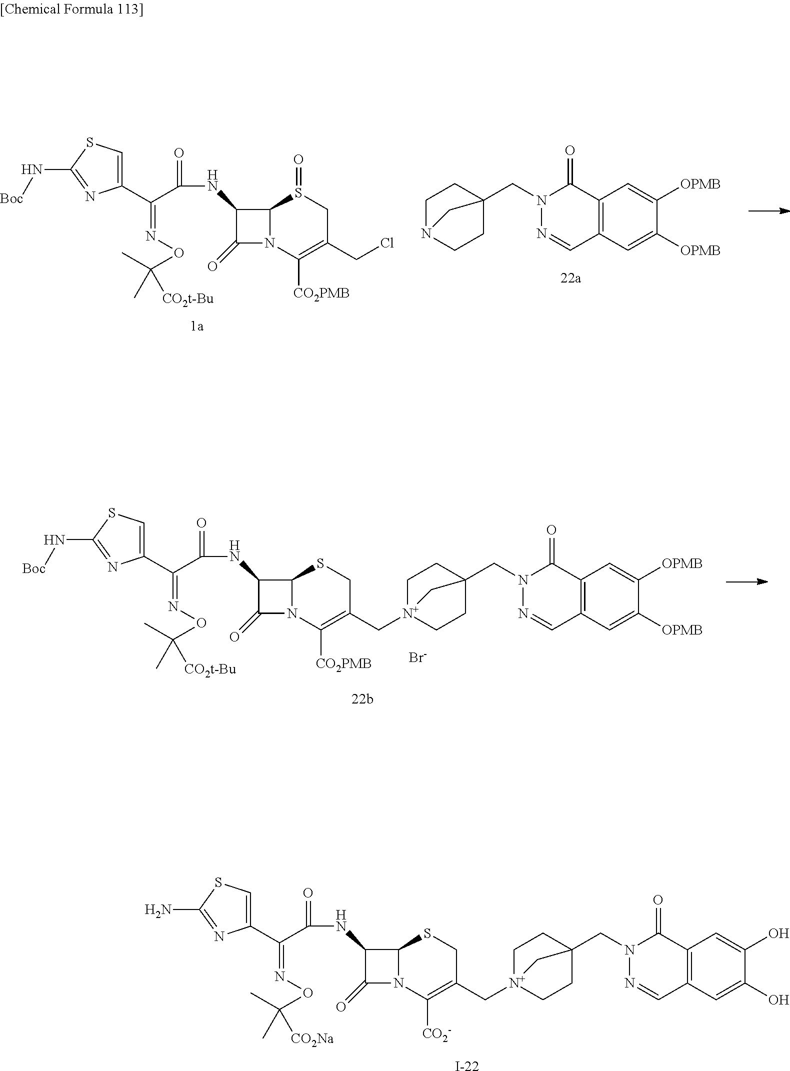

Using Compound 1a (796 mg, 1.0 mmol) and Compound 22a (528 mg, 1.0 mmol), Compound I-22 was synthesized according to the same procedure as Compound I-20. Yield: 654.3 mg, (74%)

¹H-NMR (D₂O) δ: 8.08 (1H, s), 7.38 (1H, s), 7.05 (1H, s), 6.93 (1H, s), 5.81 (1H, d, J=4.9 Hz), 5.29 (1H, d, J=4.9 Hz), 4.46 (2H, s), 4.12 (1H, d, J=13.9 Hz), 3.85 (1H, d, J=17.1 Hz), 3.64-3.41 (7H, m), 2.18 (2H, br s), 2.00 (2H, br s), 1.50 (3H, s), 1.48 (3H, s).

Elemental Analysis: C32H33N8O10S2Na(H2O)5.3

Calculated: C, 44.06; H, 5.04; N, 12.85; S, 7.35; Na, 2.64(%).

Found: C, 43.93; H, 5.03; N, 13.01; S, 7.43; Na, 2.64(%).

### Example 23

### Synthesis of Compound (I-23)

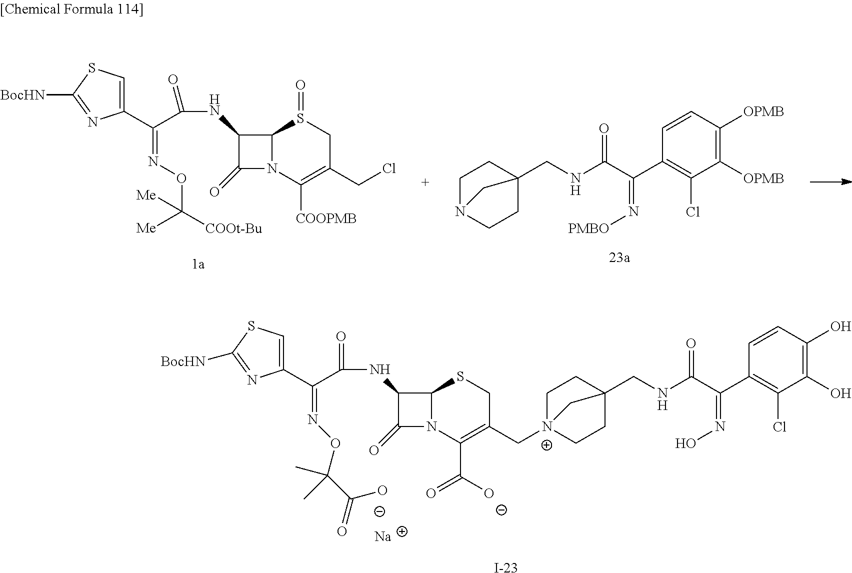

Using Compound 1a (1.53 g, 1.0 mmol) and Compound 23a (1.08 g, 1.0 mmol), Compound I-23 was synthesized according to the same procedure as Compound I-20. (Yield: 340 mg, 41%)

¹H-NMR (D₂O) δ: 7.00 (1H, s), 6.96 (1H, d, J=8.4 Hz), 6.92 (1H, d, J=8.5 Hz), 5.87 (1H, d, J=4.8 Hz), 5.36 (1H, d, J=4.9 Hz), 4.14 (1H, d, J=14.1 Hz), 3.76-3.62 (8H, m), 3.43 (1H, d, J=16.8 Hz), 3.36 (1H, d, J=8.7 Hz), 3.30 (1H, d, J=10.0 Hz), 2.22-2.20 (2H, br m), 1.97-1.94 (2H, br m), 1.51 (6H, d, J=6.0 Hz).

MS (m+1)=807.3

Additionally, the compounds described below can be synthesized according to the same procedure as described above.

TABLE 1

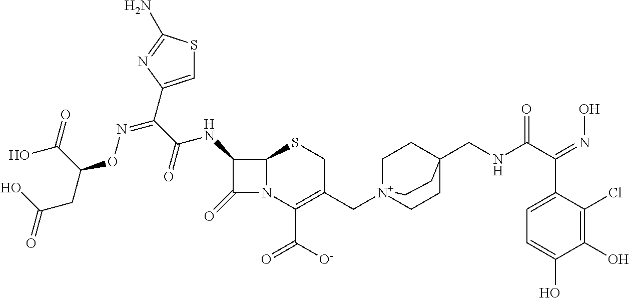 [SMILES: Nc1nc(/C(=N/O[C@@H](CC(=O)O)C(=O)O)C(=O)N[C@@H]2C(=O)N3C(C(=O)[O-])=C(C[N+]45CCC(CNC(=O)/C(=N\O)c6ccc(O)c(O)c6Cl)(CC4)CC5)CS[C@H]23)cs1]

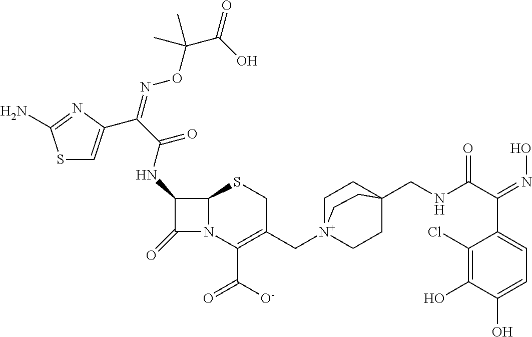 [SMILES: CC(C)(O/N=C(\C(=O)N[C@@H]1C(=O)N2C(C(=O)[O-])=C(C[N+]34CCC(CNC(=O)/C(=N\O)c5ccc(O)c(O)c5Cl)(CC3)CC4)CS[C@H]12)c1csc(N)n1)C(=O)O]

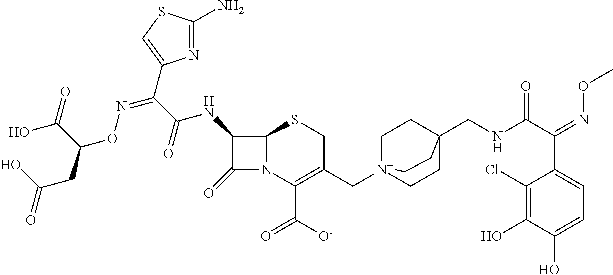 [SMILES: CO/N=C(\C(=O)NCC12CC[N+](CC3=C(C(=O)[O-])N4C(=O)[C@@H](NC(=O)/C(=N\O[C@@H](CC(=O)O)C(=O)O)c5csc(N)n5)[C@H]4SC3)(CC1)CC2)c1ccc(O)c(O)c1Cl]

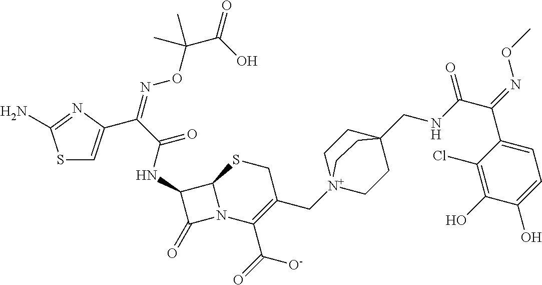 [SMILES: CO/N=C(\C(=O)NCC12CC[N+](CC3=C(C(=O)[O-])N4C(=O)[C@@H](NC(=O)/C(=N\OC(C)(C)C(=O)O)c5csc(N)n5)[C@H]4SC3)(CC1)CC2)c1ccc(O)c(O)c1Cl]

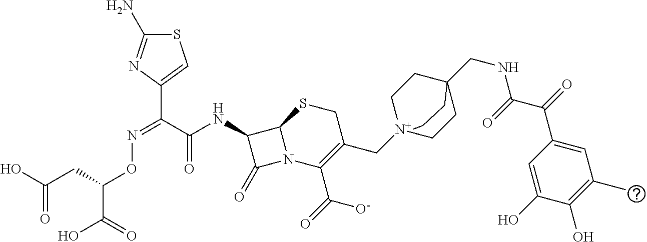 [SMILES: Cc1cc(C(=O)C(=O)NCC23CC[N+](CC4=C(C(=O)[O-])N5C(=O)[C@@H](NC(=O)/C(=N\O[C@@H](CC(=O)O)C(=O)O)c6csc(N)n6)[C@H]5SC4)(CC2)CC3)cc(O)c1O]

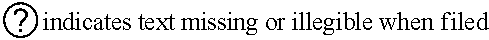

TABLE 2

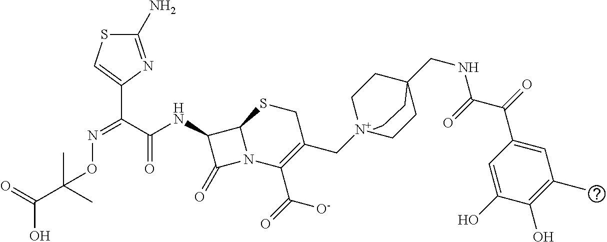 [SMILES: Cc1cc(C(=O)C(=O)NCC23CC[N+](CC4=C(C(=O)[O-])N5C(=O)[C@@H](NC(=O)/C(=N\OC(C)(C)C(=O)O)c6csc(N)n6)[C@H]5SC4)(CC2)CC3)cc(O)c1O]

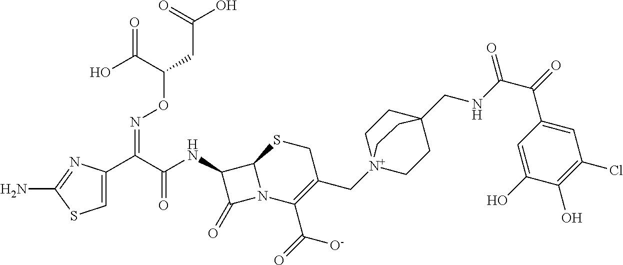 [SMILES: Nc1nc(/C(=N/O[C@@H](CC(=O)O)C(=O)O)C(=O)N[C@@H]2C(=O)N3C(C(=O)[O-])=C(C[N+]45CCC(CNC(=O)C(=O)c6cc(O)c(O)c(Cl)c6)(CC4)CC5)CS[C@H]23)cs1]

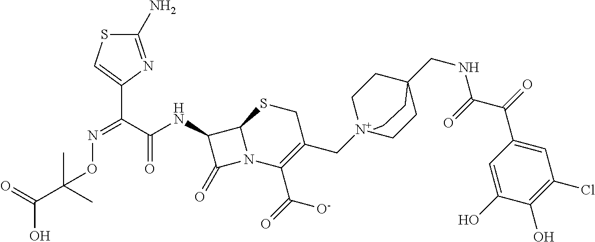 [SMILES: CC(C)(O/N=C(\C(=O)N[C@@H]1C(=O)N2C(C(=O)[O-])=C(C[N+]34CCC(CNC(=O)C(=O)c5cc(O)c(O)c(Cl)c5)(CC3)CC4)CS[C@H]12)c1csc(N)n1)C(=O)O]

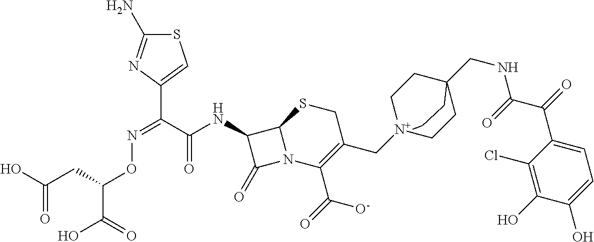 [SMILES: Nc1nc(/C(=N/O[C@@H](CC(=O)O)C(=O)O)C(=O)N[C@@H]2C(=O)N3C(C(=O)[O-])=C(C[N+]45CCC(CNC(=O)C(=O)c6ccc(O)c(O)c6Cl)(CC4)CC5)CS[C@H]23)cs1]

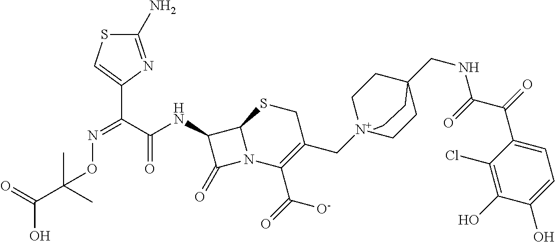 [SMILES: CC(C)(O/N=C(\C(=O)N[C@@H]1C(=O)N2C(C(=O)[O-])=C(C[N+]34CCC(CNC(=O)C(=O)c5ccc(O)c(O)c5Cl)(CC3)CC4)CS[C@H]12)c1csc(N)n1)C(=O)O]

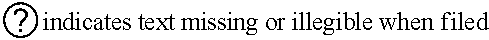

TABLE 3

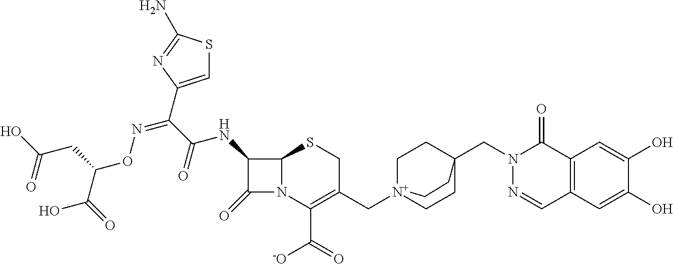

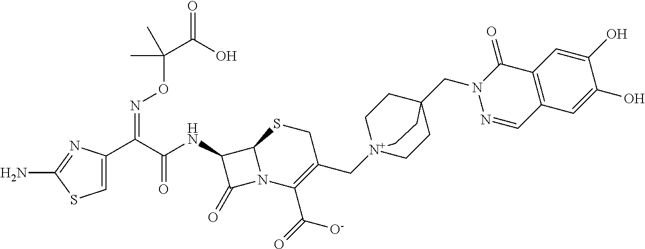 [SMILES: CC(C)(O/N=C(\C(=O)N[C@@H]1C(=O)N2C(C(=O)[O-])=C(C[N+]34CCC(Cn5ncc6cc(O)c(O)cc6c5=O)(CC3)CC4)CS[C@H]12)c1csc(N)n1)C(=O)O]

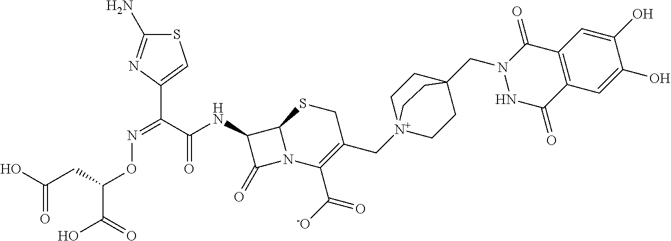

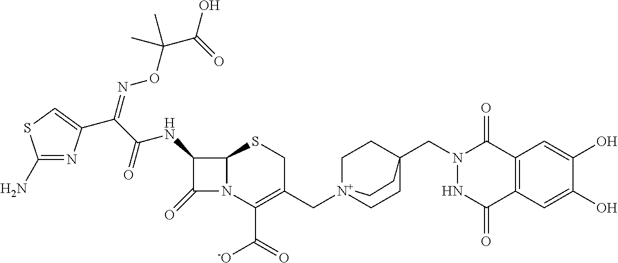

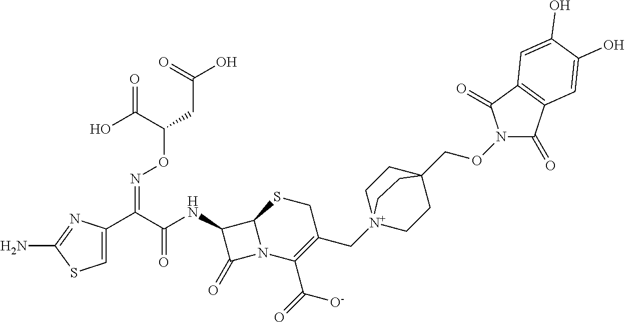 [SMILES: Nc1nc(/C(=N/O[C@@H](CC(=O)O)C(=O)O)C(=O)N[C@@H]2C(=O)N3C(C(=O)[O-])=C(C[N+]45CCC(CON6C(=O)c7cc(O)c(O)cc7C6=O)(CC4)CC5)CS[C@H]23)cs1]

TABLE 4

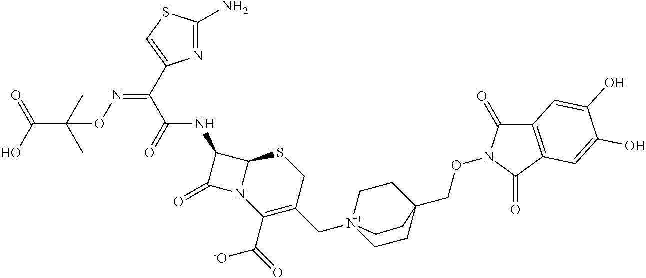

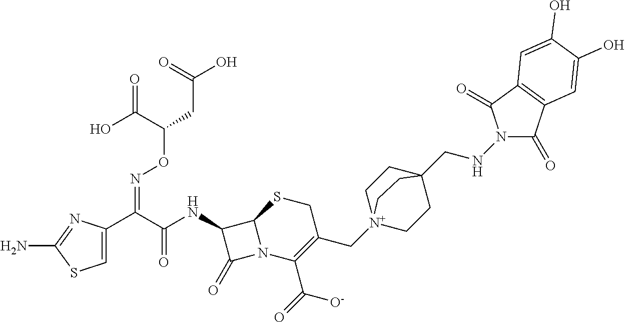 [SMILES: Nc1nc(/C(=N/O[C@@H](CC(=O)O)C(=O)O)C(=O)N[C@@H]2C(=O)N3C(C(=O)[O-])=C(C[N+]45CCC(CNN6C(=O)c7cc(O)c(O)cc7C6=O)(CC4)CC5)CS[C@H]23)cs1]

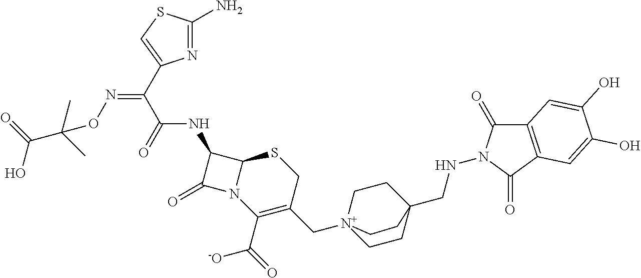

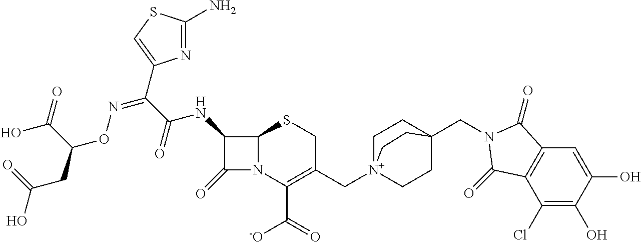

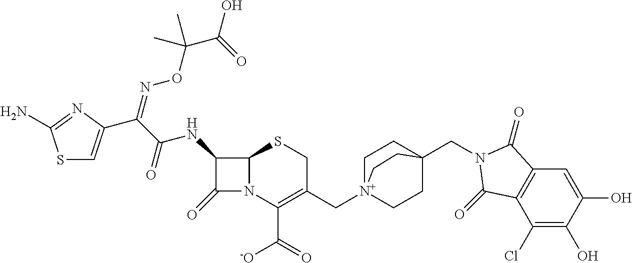

TABLE 5

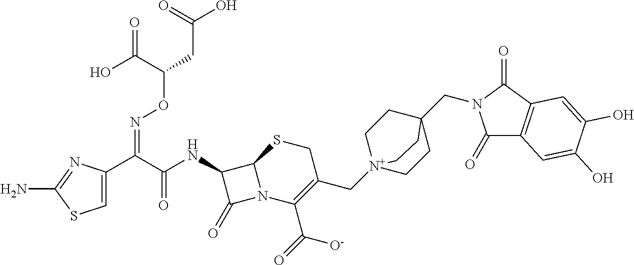 [SMILES: Nc1nc(/C(=N/O[C@@H](CC(=O)O)C(=O)O)C(=O)N[C@@H]2C(=O)N3C(C(=O)[O-])=C(C[N+]45CCC(CN6C(=O)c7cc(O)c(O)cc7C6=O)(CC4)CC5)CS[C@H]23)cs1]

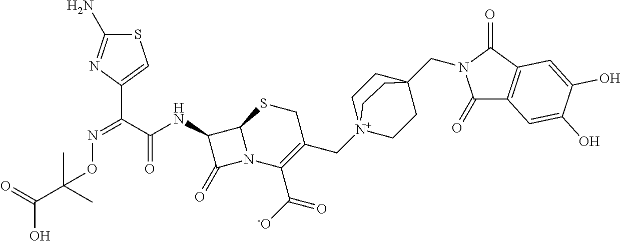

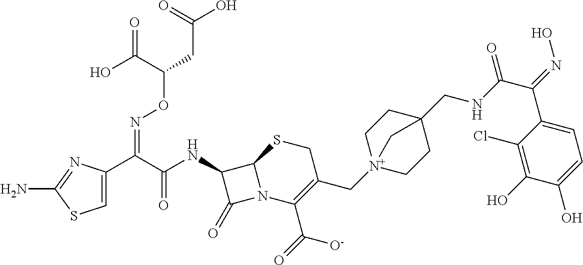 [SMILES: Nc1nc(/C(=N/O[C@@H](CC(=O)O)C(=O)O)C(=O)N[C@@H]2C(=O)N3C(C(=O)[O-])=C(C[N+]45CCC(CNC(=O)/C(=N\O)c6ccc(O)c(O)c6Cl)(CC4)C5)CS[C@H]23)cs1]

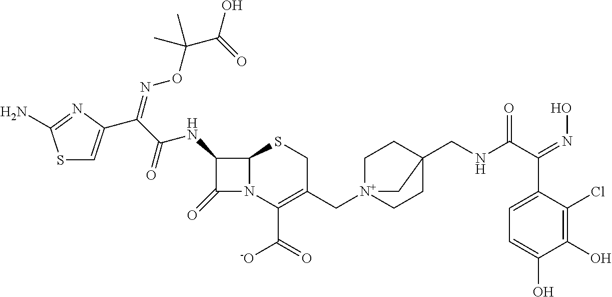

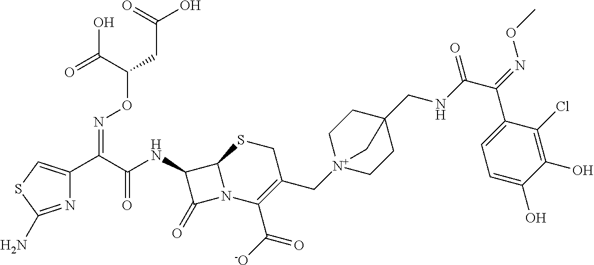

TABLE 6

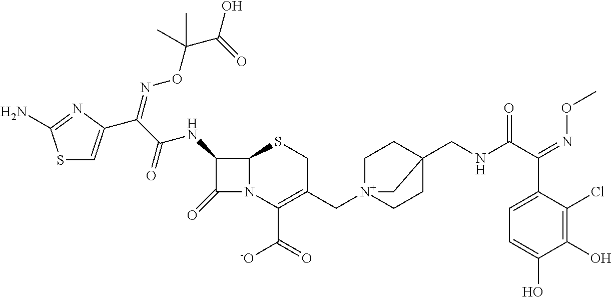

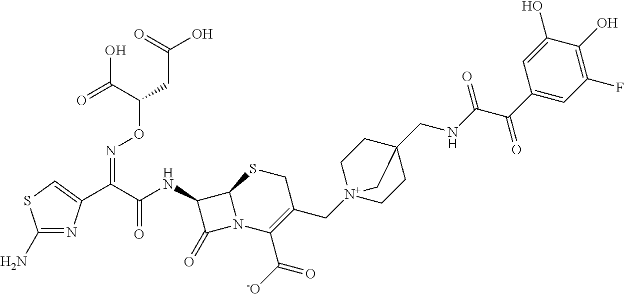

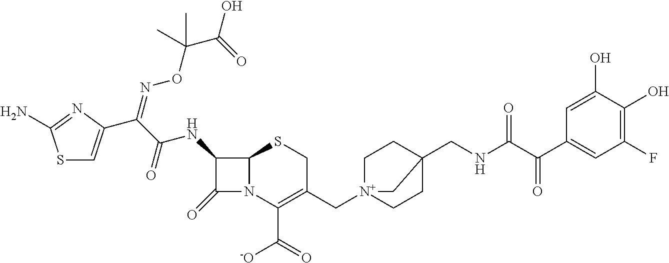

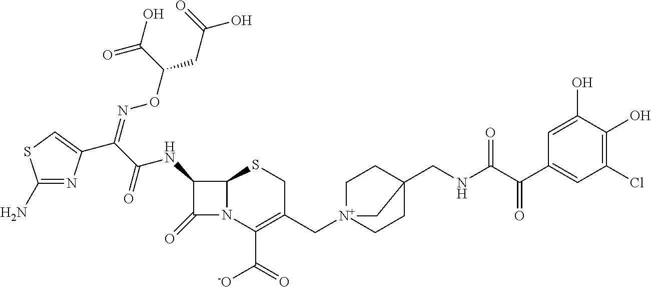

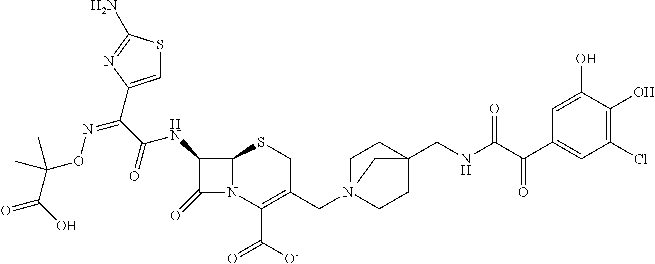 [SMILES: CC(C)(O/N=C(\C(=O)N[C@@H]1C(=O)N2C(C(=O)[O-])=C(C[N+]34CCC(CNC(=O)C(=O)c5cc(O)c(O)c(Cl)c5)(CC3)C4)CS[C@H]12)c1csc(N)n1)C(=O)O]

TABLE 7

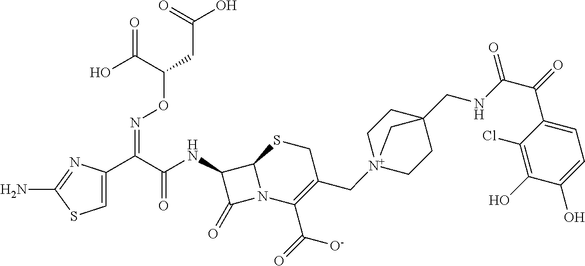 [SMILES: Nc1nc(/C(=N/O[C@@H](CC(=O)O)C(=O)O)C(=O)N[C@@H]2C(=O)N3C(C(=O)[O-])=C(C[N+]45CCC(CNC(=O)C(=O)c6ccc(O)c(O)c6Cl)(CC4)C5)CS[C@H]23)cs1]

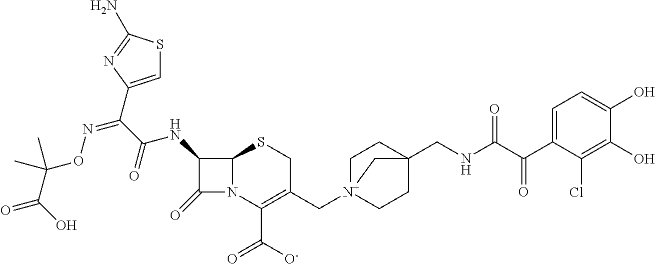 [SMILES: CC(C)(O/N=C(\C(=O)N[C@@H]1C(=O)N2C(C(=O)[O-])=C(C[N+]34CCC(CNC(=O)C(=O)c5ccc(O)c(O)c5Cl)(CC3)C4)CS[C@H]12)c1csc(N)n1)C(=O)O]

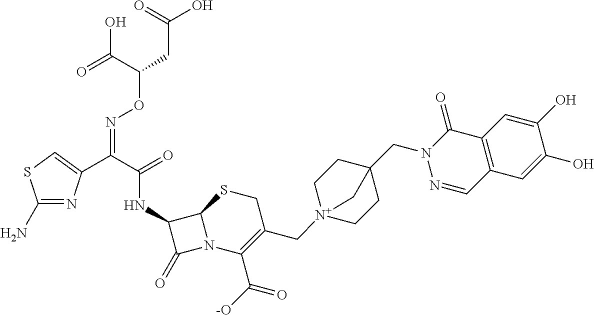

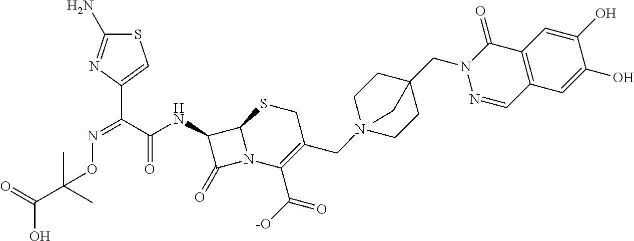

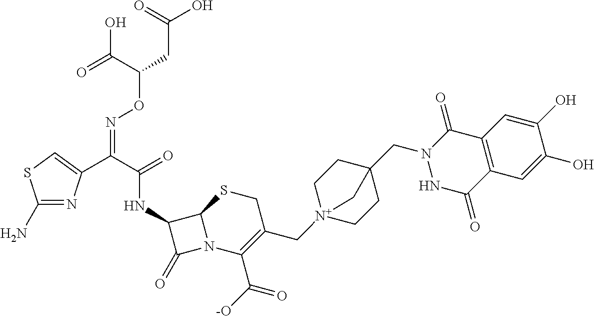

TABLE 8

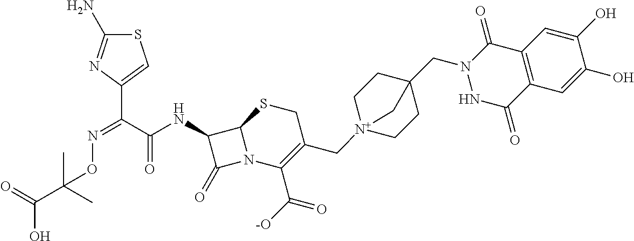

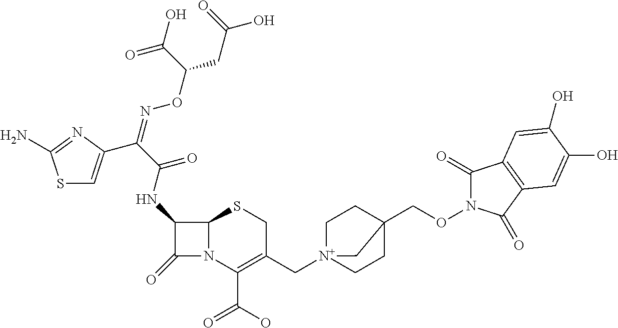 [SMILES: Nc1nc(/C(=N/O[C@@H](CC(=O)O)C(=O)O)C(=O)N[C@@H]2C(=O)N3C(C(=O)O)=C(C[N+]45CCC(CON6C(=O)c7cc(O)c(O)cc7C6=O)(CC4)C5)CS[C@H]23)cs1]

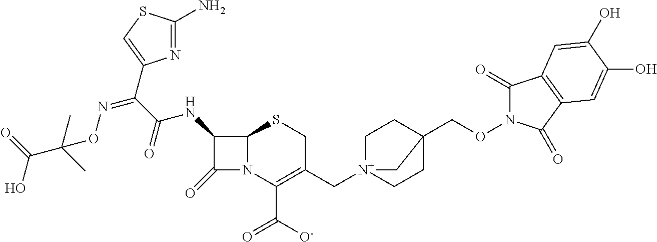 [SMILES: CC(C)(O/N=C(\C(=O)N[C@@H]1C(=O)N2C(C(=O)[O-])=C(C[N+]34CCC(CON5C(=O)c6cc(O)c(O)cc6C5=O)(CC3)C4)CS[C@H]12)c1csc(N)n1)C(=O)O]

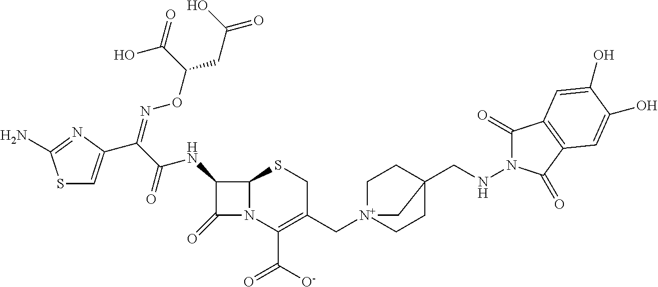 [SMILES: Nc1nc(/C(=N/O[C@@H](CC(=O)O)C(=O)O)C(=O)N[C@@H]2C(=O)N3C(C(=O)[O-])=C(C[N+]45CCC(CNN6C(=O)c7cc(O)c(O)cc7C6=O)(CC4)C5)CS[C@H]23)cs1]

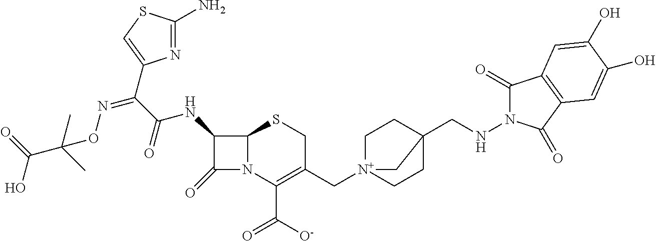 [SMILES: CC(C)(O/N=C(\C(=O)N[C@@H]1C(=O)N2C(C(=O)[O-])=C(C[N+]34CCC(CNN5C(=O)c6cc(O)c(O)cc6C5=O)(CC3)C4)CS[C@H]12)c1csc(N)n1)C(=O)O]

TABLE 9

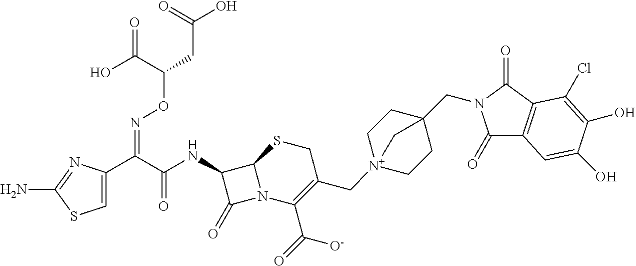 [SMILES: Nc1nc(/C(=N/O[C@@H](CC(=O)O)C(=O)O)C(=O)N[C@@H]2C(=O)N3C(C(=O)[O-])=C(C[N+]45CCC(CN6C(=O)c7cc(O)c(O)c(Cl)c7C6=O)(CC4)C5)CS[C@H]23)cs1]

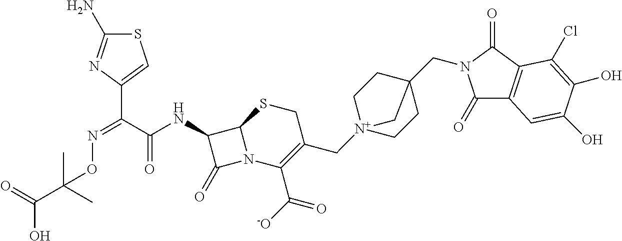

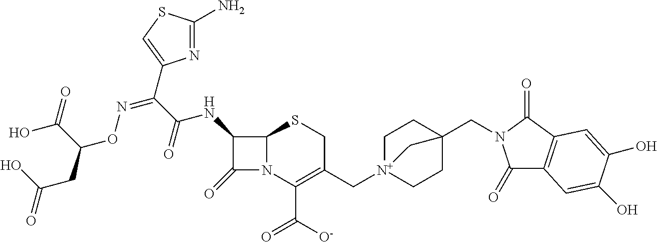 [SMILES: Nc1nc(/C(=N/O[C@@H](CC(=O)O)C(=O)O)C(=O)N[C@@H]2C(=O)N3C(C(=O)[O-])=C(C[N+]45CCC(CN6C(=O)c7cc(O)c(O)cc7C6=O)(CC4)C5)CS[C@H]23)cs1]

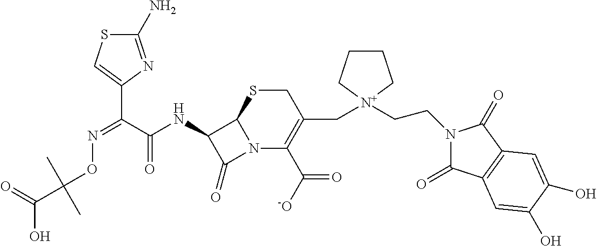

TABLE 10

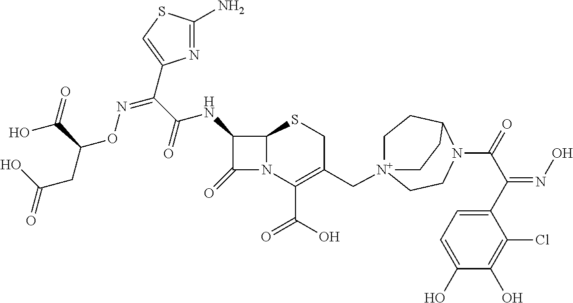 [SMILES: Nc1nc(/C(=N/O[C@@H](CC(=O)O)C(=O)O)C(=O)N[C@@H]2C(=O)N3C(C(=O)O)=C(C[N+]45CCC(CC4)N(C(=O)/C(=N\O)c4ccc(O)c(O)c4Cl)CC5)CS[C@H]23)cs1]

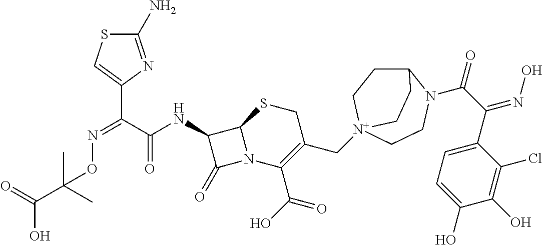 [SMILES: CC(C)(O/N=C(\C(=O)N[C@@H]1C(=O)N2C(C(=O)O)=C(C[N+]34CCC(CC3)N(C(=O)/C(=N\O)c3ccc(O)c(O)c3Cl)CC4)CS[C@H]12)c1csc(N)n1)C(=O)O]

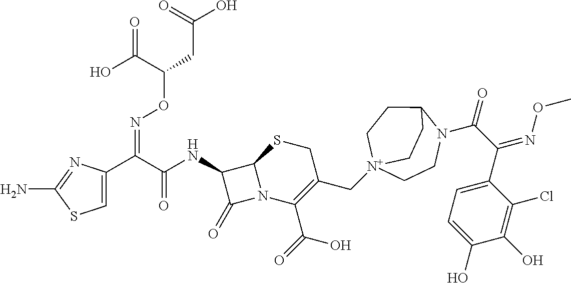 [SMILES: CO/N=C(\C(=O)N1CC[N+]2(CC3=C(C(=O)O)N4C(=O)[C@@H](NC(=O)/C(=N\O[C@@H](CC(=O)O)C(=O)O)c5csc(N)n5)[C@H]4SC3)CCC1CC2)c1ccc(O)c(O)c1Cl]

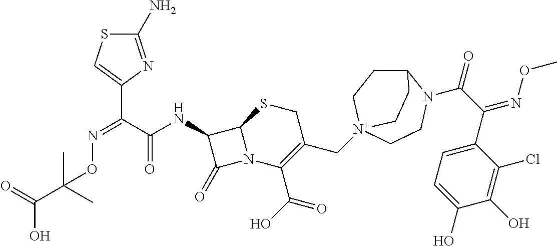 [SMILES: CO/N=C(\C(=O)N1CC[N+]2(CC3=C(C(=O)O)N4C(=O)[C@@H](NC(=O)/C(=N\OC(C)(C)C(=O)O)c5csc(N)n5)[C@H]4SC3)CCC1CC2)c1ccc(O)c(O)c1Cl]

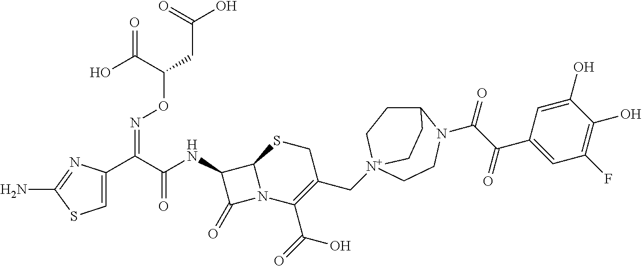 [SMILES: Nc1nc(/C(=N/O[C@@H](CC(=O)O)C(=O)O)C(=O)N[C@@H]2C(=O)N3C(C(=O)O)=C(C[N+]45CCC(CC4)N(C(=O)C(=O)c4cc(O)c(O)c(F)c4)CC5)CS[C@H]23)cs1]

TABLE 11

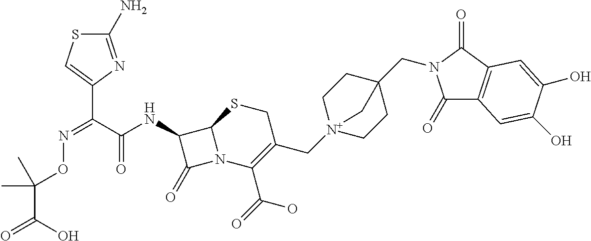 [SMILES: CC(C)(O/N=C(\C(=O)N[C@@H]1C(=O)N2C(C(=O)O)=C(C[N+]34CCC(CN5C(=O)c6cc(O)c(O)cc6C5=O)(CC3)C4)CS[C@H]12)c1csc(N)n1)C(=O)O]

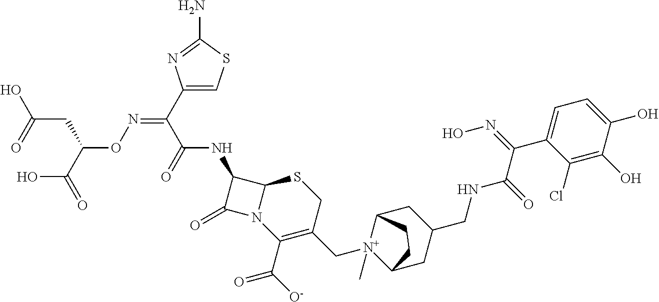 [SMILES: C[N+]1(CC2=C(C(=O)[O-])N3C(=O)[C@@H](NC(=O)/C(=N\O[C@@H](CC(=O)O)C(=O)O)c4csc(N)n4)[C@H]3SC2)[C@@H]2CC[C@H]1CC(CNC(=O)/C(=N\O)c1ccc(O)c(O)c1Cl)C2]

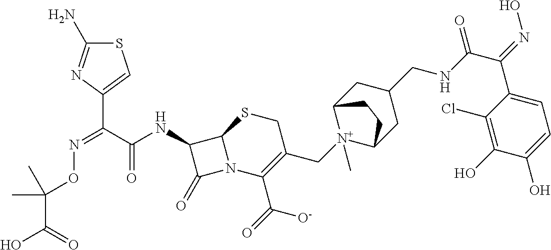 [SMILES: CC(C)(O/N=C(\C(=O)N[C@@H]1C(=O)N2C(C(=O)[O-])=C(C[N+]3(C)[C@@H]4CC[C@H]3CC(CNC(=O)/C(=N\O)c3ccc(O)c(O)c3Cl)C4)CS[C@H]12)c1csc(N)n1)C(=O)O]

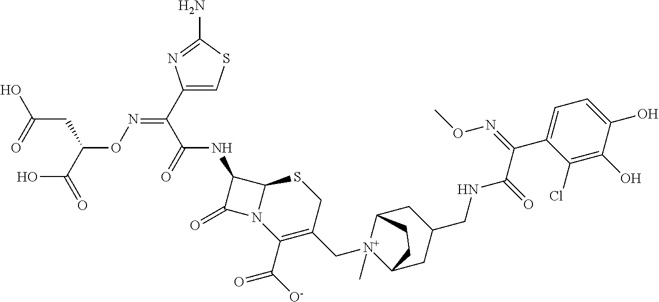 [SMILES: CO/N=C(\C(=O)NCC1C[C@H]2CC[C@@H](C1)[N+]2(C)CC1=C(C(=O)[O-])N2C(=O)[C@@H](NC(=O)/C(=N\O[C@@H](CC(=O)O)C(=O)O)c3csc(N)n3)[C@H]2SC1)c1ccc(O)c(O)c1Cl]

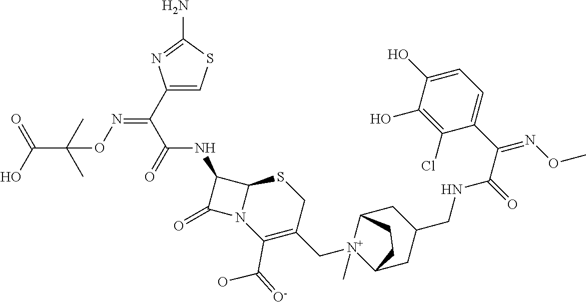

TABLE 12

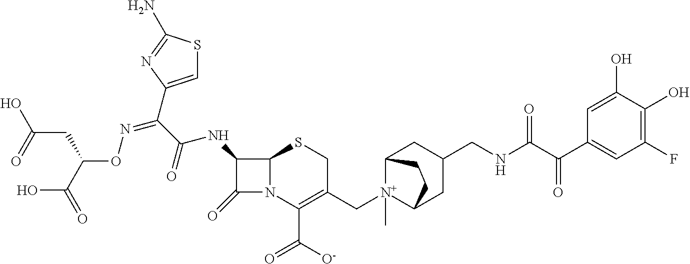 [SMILES: C[N+]1(CC2=C(C(=O)[O-])N3C(=O)[C@@H](NC(=O)/C(=N\O[C@@H](CC(=O)O)C(=O)O)c4csc(N)n4)[C@H]3SC2)[C@@H]2CC[C@H]1CC(CNC(=O)C(=O)c1cc(O)c(O)c(F)c1)C2]

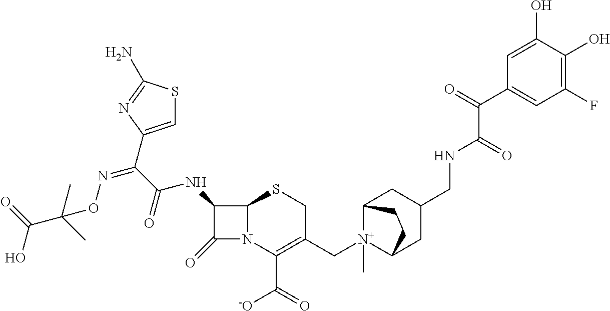

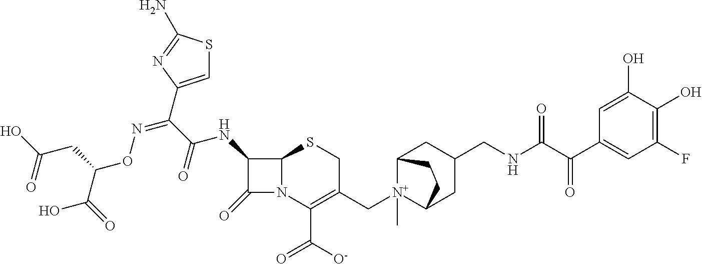 [SMILES: C[N+]1(CC2=C(C(=O)[O-])N3C(=O)[C@@H](NC(=O)/C(=N\O[C@@H](CC(=O)O)C(=O)O)c4csc(N)n4)[C@H]3SC2)[C@@H]2CC[C@H]1CC(CNC(=O)C(=O)c1cc(O)c(O)c(F)c1)C2]

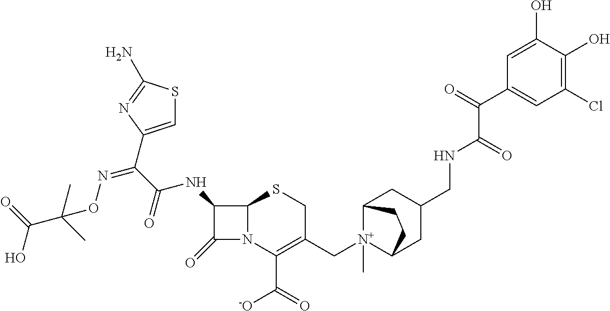

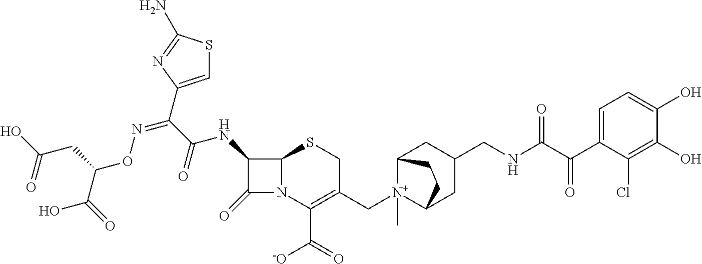

TABLE 13

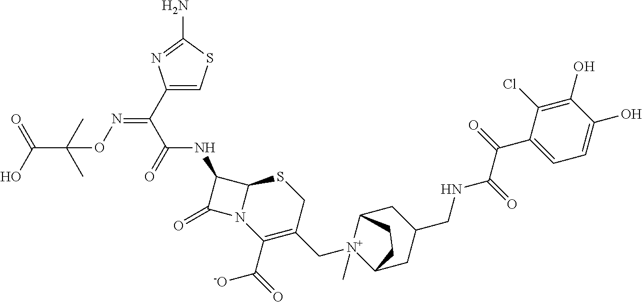

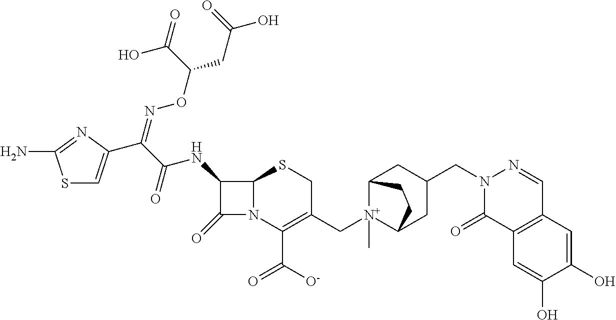 [SMILES: C[N+]1(CC2=C(C(=O)[O-])N3C(=O)[C@@H](NC(=O)/C(=N\O[C@@H](CC(=O)O)C(=O)O)c4csc(N)n4)[C@H]3SC2)[C@@H]2CC[C@H]1CC(Cn1ncc3cc(O)c(O)cc3c1=O)C2]

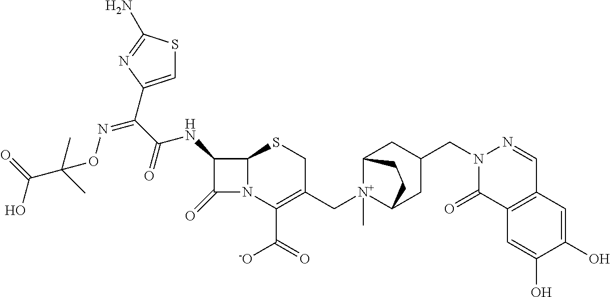

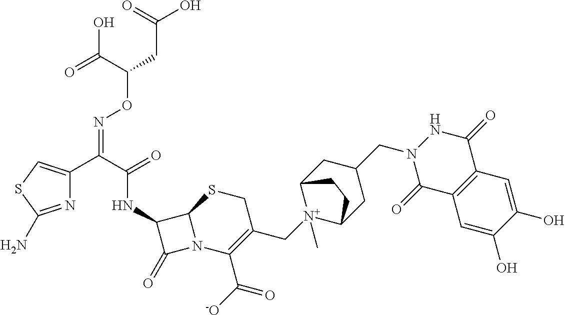

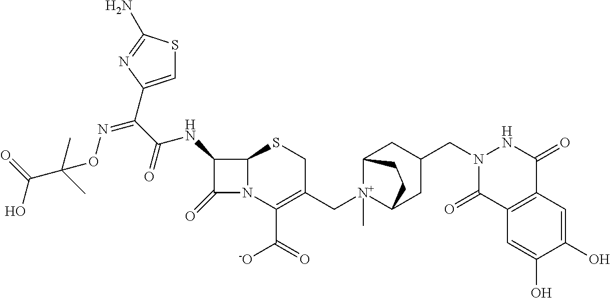

TABLE 14

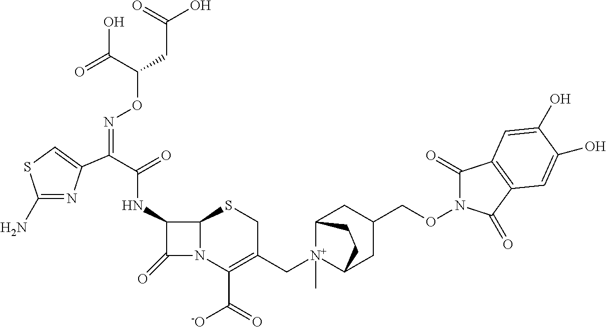

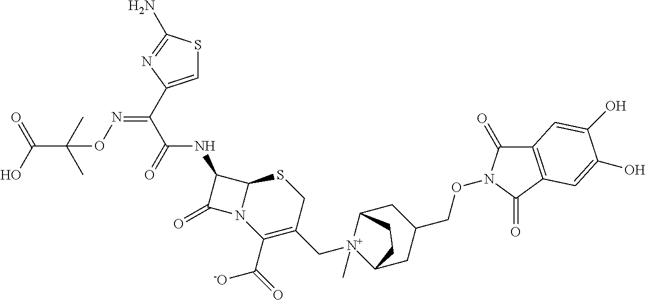

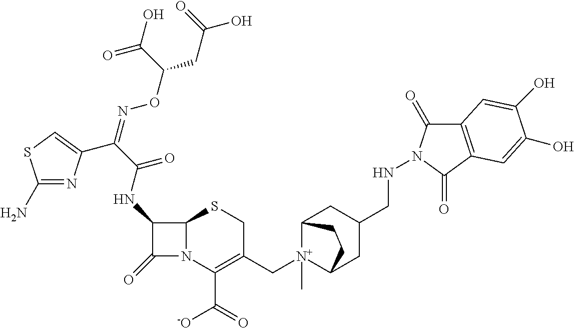

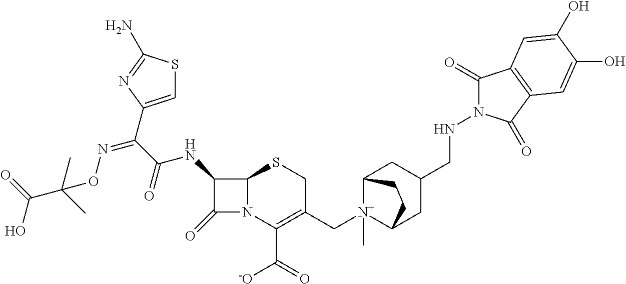

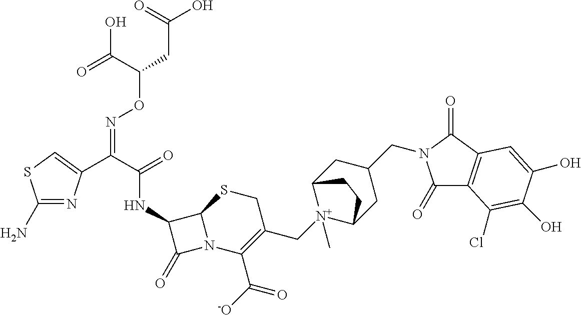

TABLE 15

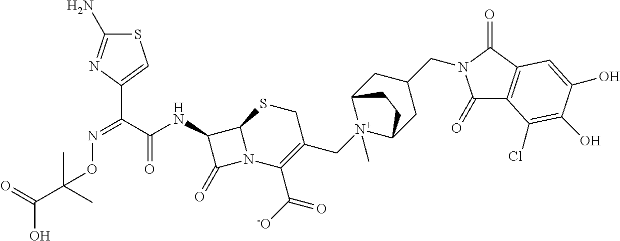

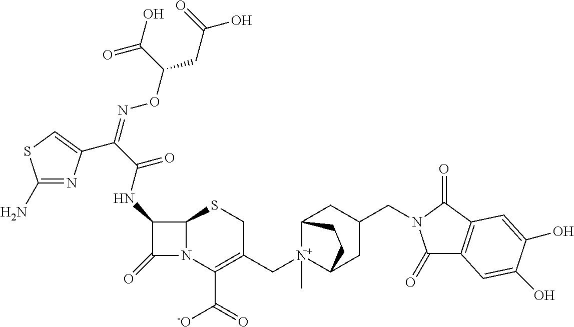

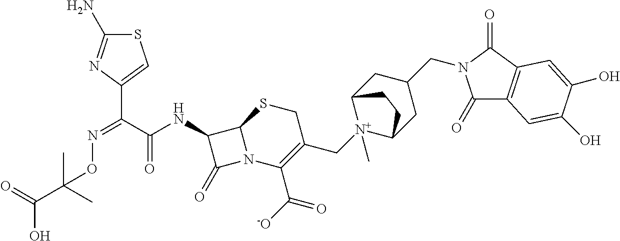

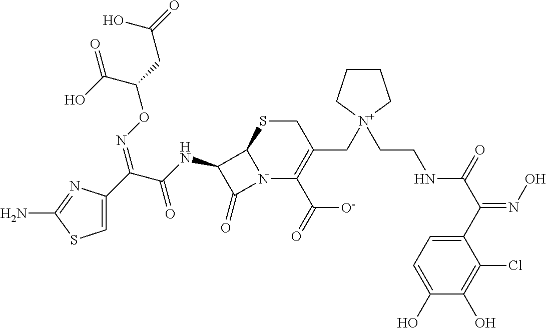 [SMILES: Nc1nc(/C(=N/O[C@@H](CC(=O)O)C(=O)O)C(=O)N[C@@H]2C(=O)N3C(C(=O)[O-])=C(C[N+]4(CCNC(=O)/C(=N\O)c5ccc(O)c(O)c5Cl)CCCC4)CS[C@H]23)cs1]

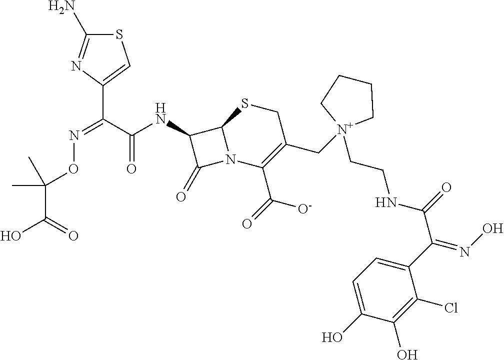 [SMILES: CC(C)(O/N=C(\C(=O)N[C@@H]1C(=O)N2C(C(=O)[O-])=C(C[N+]3(CCNC(=O)/C(=N\O)c4ccc(O)c(O)c4Cl)CCCC3)CS[C@H]12)c1csc(N)n1)C(=O)O]

TABLE 16

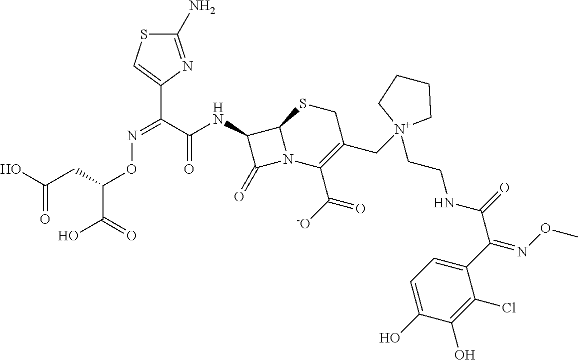

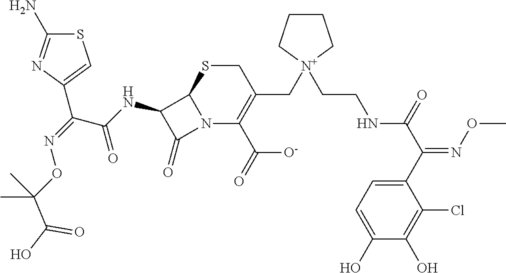 [SMILES: CO/N=C(\C(=O)NCC[N+]1(CC2=C(C(=O)[O-])N3C(=O)[C@@H](NC(=O)/C(=N\OC(C)(C)C(=O)O)c4csc(N)n4)[C@H]3SC2)CCCC1)c1ccc(O)c(O)c1Cl]

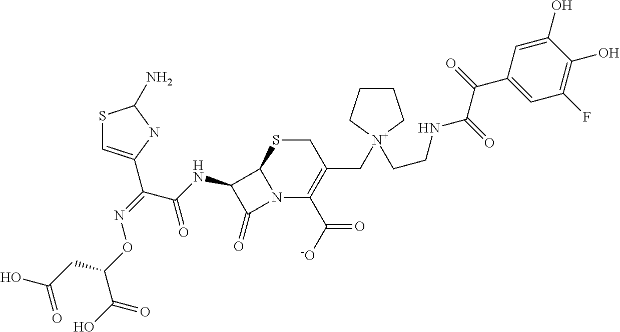

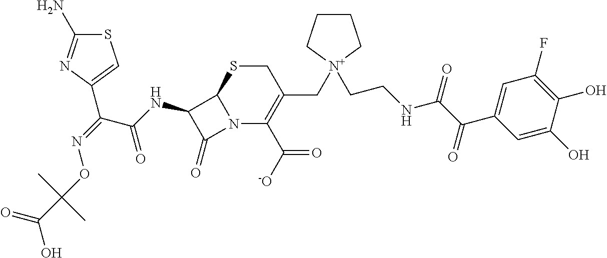

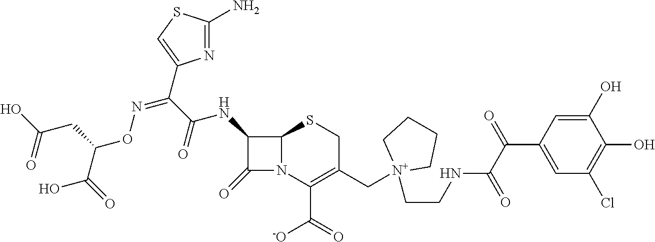

TABLE 17

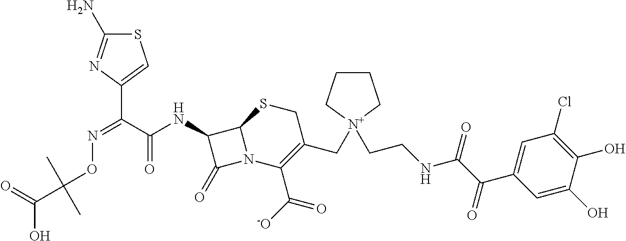

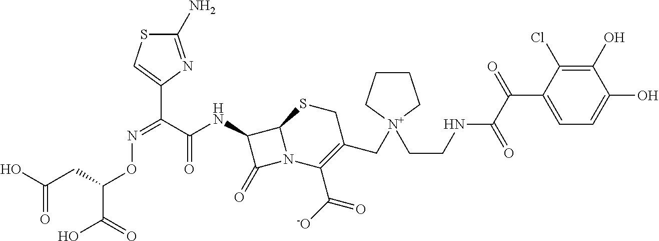

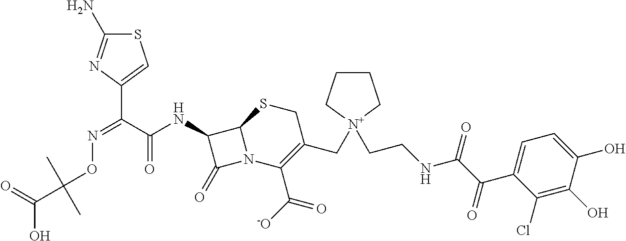

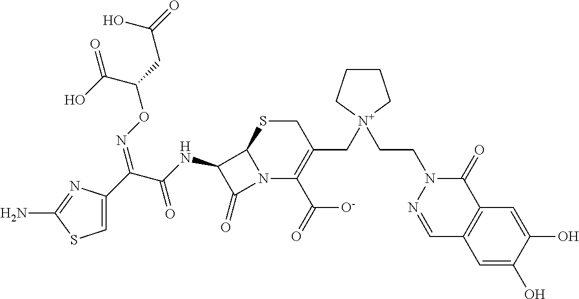 [SMILES: Nc1nc(/C(=N/O[C@@H](CC(=O)O)C(=O)O)C(=O)N[C@@H]2C(=O)N3C(C(=O)[O-])=C(C[N+]4(CCn5ncc6cc(O)c(O)cc6c5=O)CCCC4)CS[C@H]23)cs1]

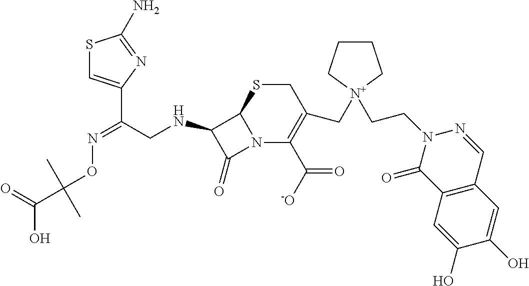

TABLE 18

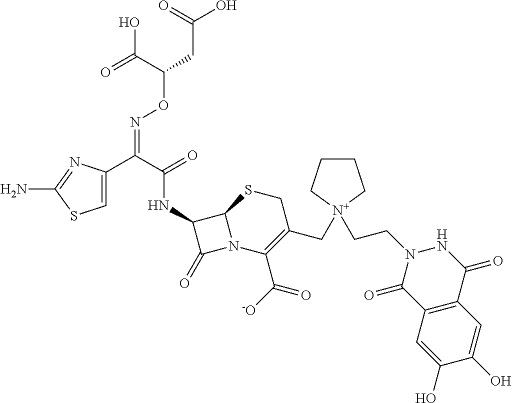

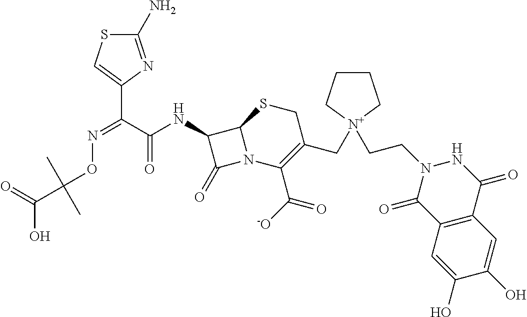

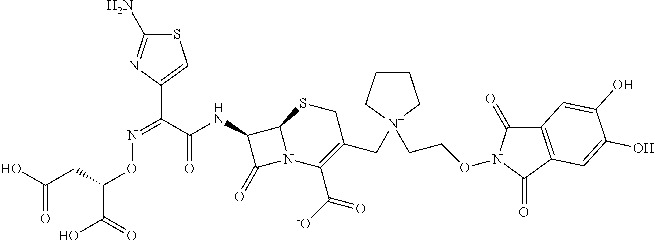

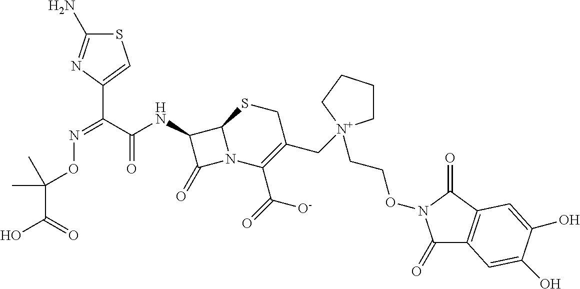 [SMILES: CC(C)(O/N=C(\C(=O)N[C@@H]1C(=O)N2C(C(=O)[O-])=C(C[N+]3(CCON4C(=O)c5cc(O)c(O)cc5C4=O)CCCC3)CS[C@H]12)c1csc(N)n1)C(=O)O]

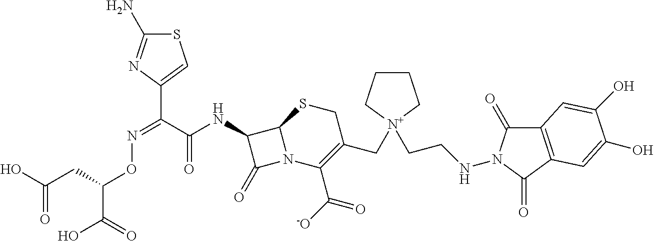

TABLE 19

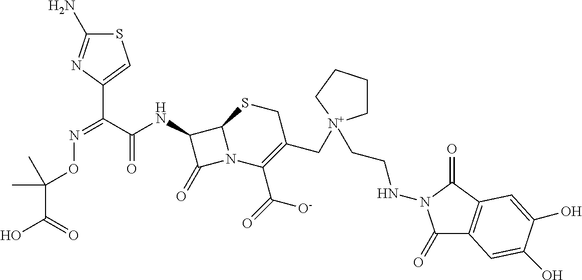 [SMILES: CC(C)(O/N=C(\C(=O)N[C@@H]1C(=O)N2C(C(=O)[O-])=C(C[N+]3(CCNN4C(=O)c5cc(O)c(O)cc5C4=O)CCCC3)CS[C@H]12)c1csc(N)n1)C(=O)O]

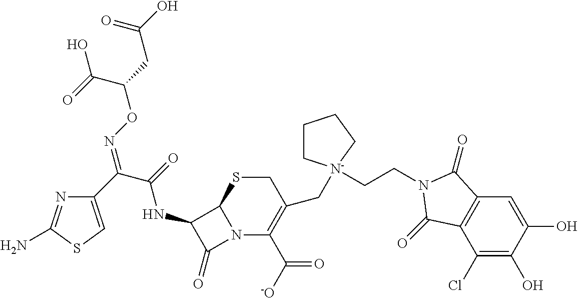

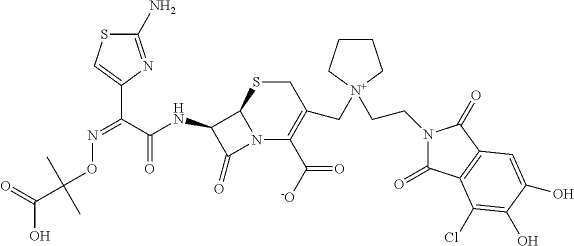

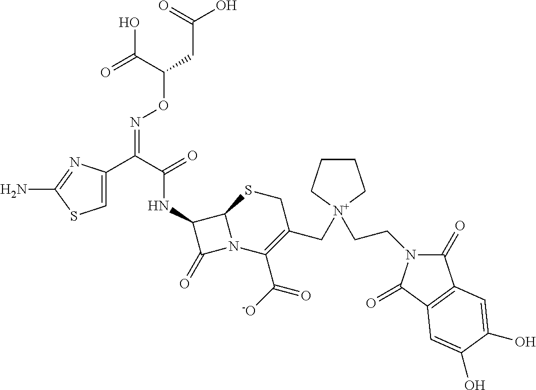

TABLE 20

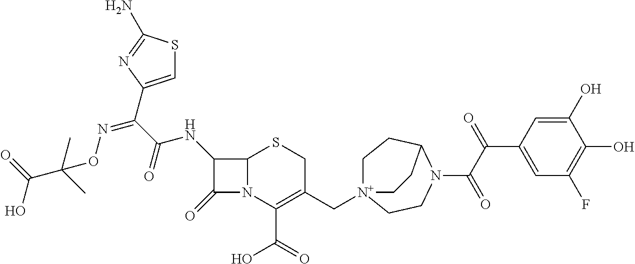 [SMILES: CC(C)(O/N=C(\C(=O)NC1C(=O)N2C(C(=O)O)=C(C[N+]34CCC(CC3)N(C(=O)C(=O)c3cc(O)c(O)c(F)c3)CC4)CSC12)c1csc(N)n1)C(=O)O]

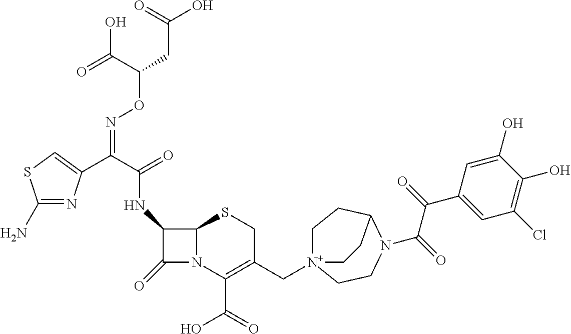 [SMILES: Nc1nc(/C(=N/O[C@@H](CC(=O)O)C(=O)O)C(=O)N[C@@H]2C(=O)N3C(C(=O)O)=C(C[N+]45CCC(CC4)N(C(=O)C(=O)c4cc(O)c(O)c(Cl)c4)CC5)CS[C@H]23)cs1]

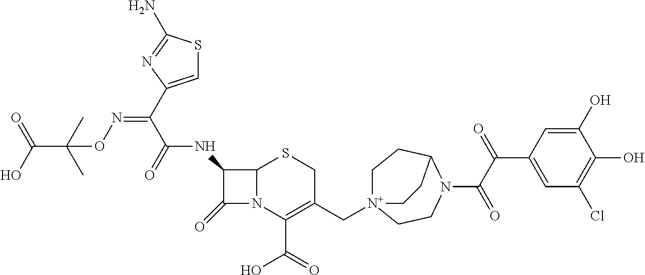 [SMILES: CC(C)(O/N=C(\C(=O)N[C@@H]1C(=O)N2C(C(=O)O)=C(C[N+]34CCC(CC3)N(C(=O)C(=O)c3cc(O)c(O)c(Cl)c3)CC4)CSC12)c1csc(N)n1)C(=O)O]

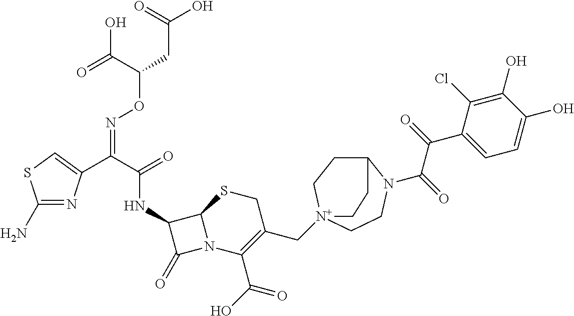 [SMILES: Nc1nc(/C(=N/O[C@@H](CC(=O)O)C(=O)O)C(=O)N[C@@H]2C(=O)N3C(C(=O)O)=C(C[N+]45CCC(CC4)N(C(=O)C(=O)c4ccc(O)c(O)c4Cl)CC5)CS[C@H]23)cs1]

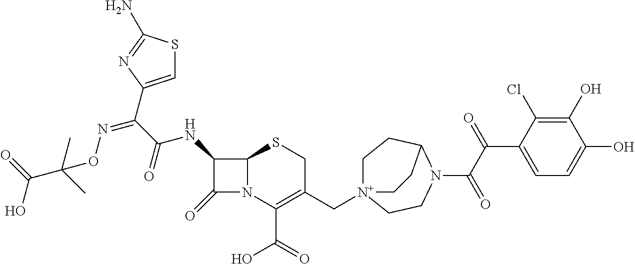 [SMILES: CC(C)(O/N=C(\C(=O)N[C@@H]1C(=O)N2C(C(=O)O)=C(C[N+]34CCC(CC3)N(C(=O)C(=O)c3ccc(O)c(O)c3Cl)CC4)CS[C@H]12)c1csc(N)n1)C(=O)O]

### Experimental Example

Compound (I) of the present invention was evaluated for in vitro antimicrobial activity thereof.

### (Test Methods)

Measurement of Minimum Inhibitory Concentration (MIC: μg/mL) was conducted according to CLSI (Clinical and Laboratory Standards Institute) method, and the amount of bacteria for inoculation was 5×10⁵ cfu/mL, and cation-adjusted Iso-Sensitest broth containing human Apo-transferrin was used as a test medium, and the experiment was conducted using broth microdilution method.

TABLE 21

| | | Strain | Enzyme | |
|---|---|---|---|---|
| No. | Species | Name | Produced | Strain Type |
| 1 | E. Coli | ATCC | TEM-10 | ESBL producing strain |
| | | BAA-196 | | |
| 2 | E. Coli | ATCC | SHV-4 | ESBL producing strain |
| | | BAA-200 | | |
| 3 | E. cloacae | 1481026 | | |
| 4 | P. aeruginosa | 378057 | | |
| 5 | A. baumannii | 1484820 | | |

### (Results)

The test result is shown in Table 22. In the table, the values of inhibitory activity are expressed in microgram/mL (μg/mL).

TABLE 22

| | E. Coli | | | | |
|---|---|---|---|---|---|
| | ATCC | ATCC | | | |
| Exam- | BAA- | BAA- | E. cloacae | P. aeruginosa | A. baumannii |
| ple No. | 196 | 200 | 1481026 | 378057 | 1484820 |
| I-1 | 0.125 | 0.25 | 1 | ≦0.031 | 0.5 |
| I-2 | 0.25 | 0.25 | 16 | ≦0.031 | 2 |
| I-3 | 0.125 | 0.125 | | 0.063 | 0.25 |
| I-4 | 0.125 | 0.25 | 1 | ≦0.031 | 0.25 |
| I-5 | 0.25 | 0.25 | 16 | ≦0.031 | 1 |
| I-6 | 0.25 | 0.25 | | 0.063 | 4 |
| I-7 | 0.063 | 0.125 | 0.5 | ≦0.031 | 0.25 |
| I-8 | 0.125 | 0.125 | 2 | 0.063 | 0.125 |
| I-9 | 0.125 | 0.25 | 4 | 0.063 | 0.5 |
| I-10 | 0.063 | 0.125 | 2 | ≦0.031 | 0.25 |
| I-11 | 0.063 | 0.125 | 0.5 | ≦0.031 | 0.063 |
| I-12 | 0.125 | 0.25 | 4 | ≦0.031 | 1 |
| I-13 | ≦0.031 | 0.063 | 0.5 | 0.063 | 0.125 |
| I-14 | ≦0.031 | 0.063 | 0.5 | ≦0.031 | 0.125 |
| I-15 | 0.125 | 0.125 | 0.5 | 0.063 | 0.25 |
| I-16 | ≦0.031 | 0.063 | 1 | 0.063 | 0.125 |
| I-17 | 0.063 | 0.063 | 0.5 | ≦0.031 | 0.5 |
| I-18 | 0.063 | 0.063 | 0.5 | ≦0.031 | 0.25 |
| I-19 | 0.063 | 0.063 | 0.5 | ≦0.031 | 0.25 |
| I-20 | 0.063 | 0.25 | 2 | ≦0.031 | 4 |
| I-21 | 0.063 | 0.25 | | ≦0.031 | 1 |
| I-22 | ≦0.031 | 0.125 | 1 | ≦0.031 | 1 |
| I-23 | 0.125 | 0.5 | 4 | ≦0.031 | 2 |

As shown in the above results, Compound (I) of the present invention have a wide antimicrobial spectrum, in particular, potent antimicrobial spectrum against Gram negative bacteria, and/or effectiveness against multidrug-resistant bacteria, and further to exhibit high stability against beta-lactamase producing Gram negative bacteria.

### Formulation Example 1

Powder of a compound of the present invention is formulated to prepare an injecting agent.

### INDUSTRIAL APPLICABILITY

The compounds of the present invention have a wide antimicrobial spectrum against Gram negative bacteria and Gram positive bacteria, and are effective as an antimicrobial drug having high stability against beta-lactamase producing Gram negative bacteria. Moreover, the present compounds have good disposition, and high water solubility, and thus particularly effective as an injecting agent.

1. A compound of Formula (I): 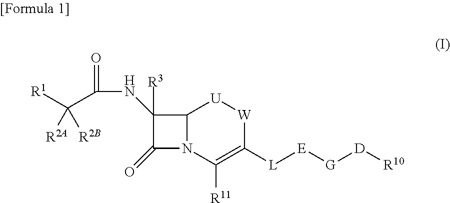
or an amino-protected compound when the amino group is present on the ring in the 7-side chain, or a pharmaceutically acceptable salt thereof,
wherein
W is —CH₂—,—S— or—O—,
a) U is —CH₂—, —S—, —S(═O)— or —O—, when W is —CH₂—; or,
b) U is —CH₂—, when W is —S— or —O—;
L is substituted or unsubstituted lower alkylene or substituted or unsubstituted lower alkenylene;
R¹ is substituted or unsubstituted carbocyclyl or substituted or unsubstituted heterocyclyl;
with regard to R^{2A} and R^{2B},
a) R^{2A} is a hydrogen atom, substituted or unsubstituted amino, —SO₃H, substituted or unsubstituted amino sulfonyl, carboxy, substituted or unsubstituted lower alkyloxycarbonyl, substituted or unsubstituted carbamoyl, hydroxy, or substituted carbonyloxy; and R^{2B} is a hydrogen atom, or
b) R^{2A} and R^{2B} are taken together to form substituted or unsubstituted methylidene, or substituted or unsubstituted hydroxyimino;
R³ is a hydrogen atom, —OCH₃ or —NH—CH(═O);
R¹ is carboxylate anion (—COO⁻) or a bioisoster of carboxylate anion (—COO⁻);
E is a substituted or unsubstituted divalent group containing quaternary ammonium ion(s);
G is a single bond, substituted or unsubstituted lower alkylene, substituted or unsubstituted lower alkenylene, or substituted or unsubstituted lower alkynylene;
D is a single bond, —C(═O)—, —O—C(═O)—, —C(═O)—O—, —NR⁶—C(═O)—, —C(═O)—NR⁶—, —C(═O)—C(═O)—NR⁶—, —NR⁶—C(═O)—C(═O)—, —C(═O)—NR⁶—C(═O)—, —C(═O)—C(═O)—, —O—, —NR⁶—NR⁶—C(═O)—, —C(═O)—NR⁶—NR⁶—, —N═N—C(═O)—, —C(═O)—N═N—, —C═N—NR⁶—C(═O)—, —C═N—C(═O)—, —N═CR⁶—C(═O)—, —C═N—C(═O)—NR⁶—, —NR⁶—C(═O)—C(═N—OR⁶)—, —C(═N—OR⁶)—C(═O)—NR⁶—, —NR⁶—C(═N—OR⁶)—, —C(═N—OR⁶)—NR⁶—, —C(═O)—C(═N—OR⁶)—, —C(═N—OR⁶)—C(═O)—, —S—, —S(═O)—, —S(═O)₂—NR⁶—, —NR⁶—S(═O)₂—, —NR⁶—CH₂—, —CH₂—NR⁶—, —S(═O)₂— or —NR⁶—;
R⁶ is independently a hydrogen atom or substituted or unsubstituted lower alkyl;
R¹⁰ is
1) substituted or unsubstituted phenyl or substituted or unsubstituted 6-membered heterocyclyl containing 1-3 nitrogen atoms;
2) substituted or unsubstituted 9-membered bicyclic aromatic heterocyclyl; or
3) a group represented by Formula: 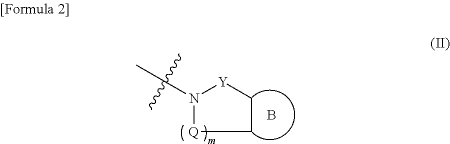
wherein
ring B is substituted or unsubstituted carbocycle or substituted or unsubstituted heterocycle;
Y is —C(═O)— or —S(═O)₂—;
Q is independently —O—, —S—, —NR—, —CR⁸R⁹—, —C(═O)—, —S(═O)₂— or —N═CH—;
R⁸ and R⁹ are each independently a hydrogen atom or substituted or unsubstituted lower alkyl;
m is an integer from 1 to 3,
provided that,
a) D is —C(═O)—C(═O)—NR⁶—, —NR⁶—C(═O)—C(═O)—, —C(═O)—NR⁶—C(═O)—, —NR⁶—NR⁶—C(═O)—, —C(═O)—NR⁶—NR⁶—, —N═N—C(═O)—, —C(═O)—N═N—, —C═N—NR⁶—C(═O)—, —C═N—C(═O)—, —N═CR⁶—C(═O)—, —C═N—C(═O)—NR⁶—, —NR⁶—C(═O)—C(═N—OR⁶)—, —C(═N—OR⁶)—C(═O)—NR⁶—, —NR⁶—C(═N—OR⁶)—, —C(═N—OR⁶)—NR⁶—, —C(═O)—C(═N—OR⁶)—, —C(═N—OR⁶)—C(═O)—, or —C(═O)—C(═O)—, when R¹⁰ is the above 1);
b) R¹⁰ is not a group represented by Formula: 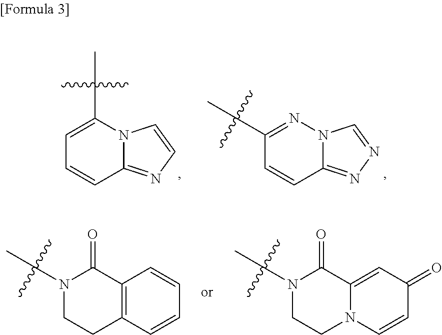 [SMILES: *c1cccc2nccn12 ; *c1ccc2nncn2n1 ; *N1CCc2ccccc2C1=O ; *N1CCn2ccc(=O)cc2C1=O]
wherein the above ring is optionally substituted by one or more substituents selected from hydroxy, chloro, fluoro, bromo, carboxy and methoxy; and
c) the compounds
wherein R¹⁰ is the above 2) or 3),
E is the group represented by Formula: 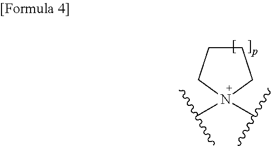
wherein p is an integer form 1 to 3, and
D is —NR⁶—C(═O)—, are excluded.

2. The compound, or an amino-protected compound when the amino is present on the ring in the 7-side chain, or a pharmaceutically acceptable salt thereof according to claim 1, wherein D is a single bond, —C(═O)—, —O—C(═O)—, —C(═O)—O—, —C(═O)—C(═O)—NR⁶—, —NR⁶—C(═O)—C(═O)—, —C(═O)—NR⁶—C(═O)—, —C(═O)—C(═O)—, —O—, —NR⁶—NR⁶—C(═O)—, —C(═O)—NR⁶—NR⁶—, —N═N—C(═O)—, —C(═O)—N═N—, —C═N—NR⁶—C(═O)—, —C═N—C(═O)—, —N═CR⁶—C(═O)—, —C═N—C(═O)—NR⁶—, —NR⁶—C(═O)—C(═N—OR⁶)—, —C(═N—OR⁶)—C(═O)—NR⁶—, —NR⁶—C(═N—OR⁶)—, —C(═N—OR⁶)—NR⁶—, —C(═O)—C(═N—OR⁶)—, —C(═N—OR⁶)—C(═O)—, —S—, —S(═O)—, —S(═O)₂—NR⁶—, —NR⁶—S(═O)₂—, —NR⁶—CH₂—, —CH₂—NR⁶—, —S(═O)₂—, or —NR⁶—.

3. The compound, or an amino-protected compound when the amino is present on the ring in the 7-side chain, or a pharmaceutically acceptable salt thereof according to claim 1, wherein R¹⁰ is the above 1), and D is —C(═O)—C(═O)—NR⁶—, —NR⁶—C(═O)—C(═O)—, —C(═O)—NR⁶—C(═O)—, —NR⁶—NR⁶—C(═O)—, —C(═O)—NR⁶—NR⁶—, —N═N—C(═O)—, —C(═O)—N═N—, —C═N—NR⁶—C(═O)—, —C═N—C(═O)—, —N═CR⁶—C(═O)—, —C═N—C(═O)—NR⁶—, —NR⁶—C(═O)—C(═N—OR⁶)—, —C(═N—OR⁶)—C(═O)—NR⁶—, —NR⁶—C(═N—OR⁶)—, —C(═N—OR⁶)—NR⁶—, —C(═O)—C(═N—OR⁶)—, —C(═N—OR⁶)—C(═O)—, or —C(═O)—C(═O)—.

4. The compound, or an amino-protected compound when the amino is present on the ring in the 7-side chain, or a pharmaceutically acceptable salt thereof according to claim 3, wherein D-R¹⁰ is the group represented by Formula: 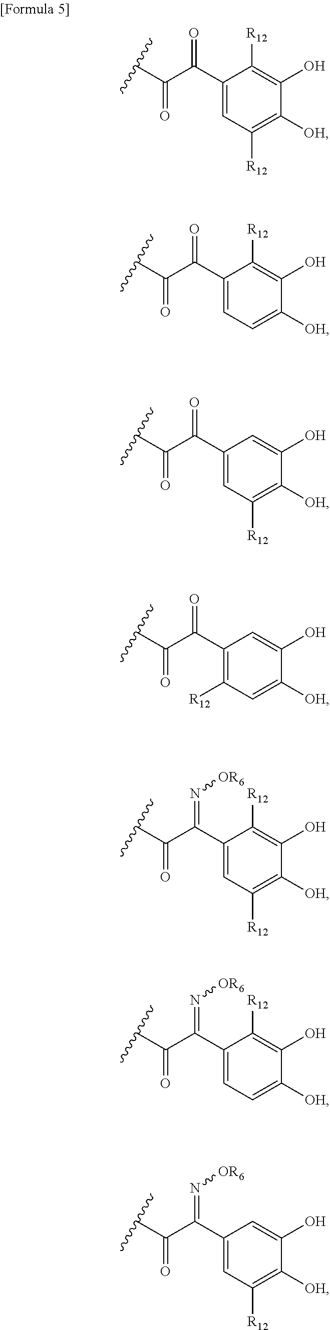 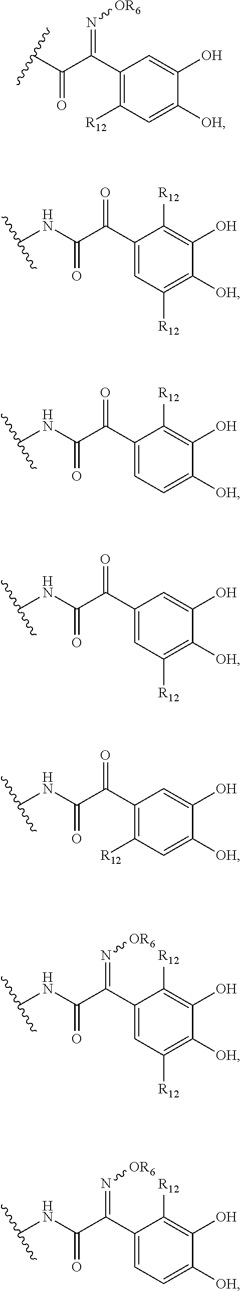 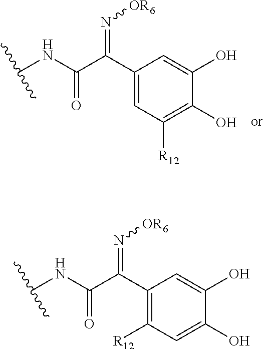
wherein
each R¹² is independently a hydrogen atom, halogen, hydroxy, —CN, —C(═O)—R¹⁵, —C(═O)—OH, —C(═O)—OR¹⁵ or OR¹⁵,
each R¹⁵ is independently lower alkyl or halo lower alkyl,
each R⁶ is independently a hydrogen atom or substituted or unsubstituted lower alkyl,
the bond of wavy line means that the bond is cis-configuration, trans-configuration or the mixture thereof.

5. The compound, or an amino-protected compound when the amino is present on the ring in the 7-side chain, or a pharmaceutically acceptable salt thereof according to claim 3, wherein D-R¹⁰ is the group represented by Formula: 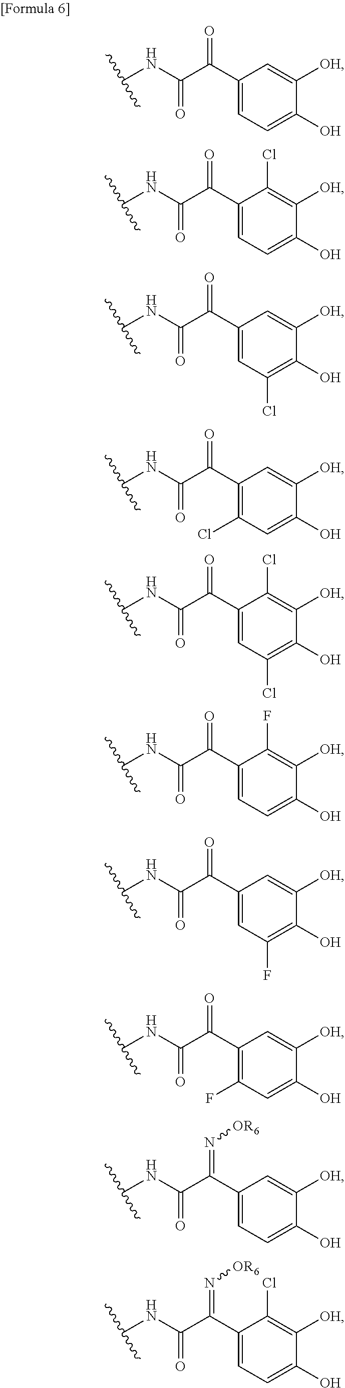 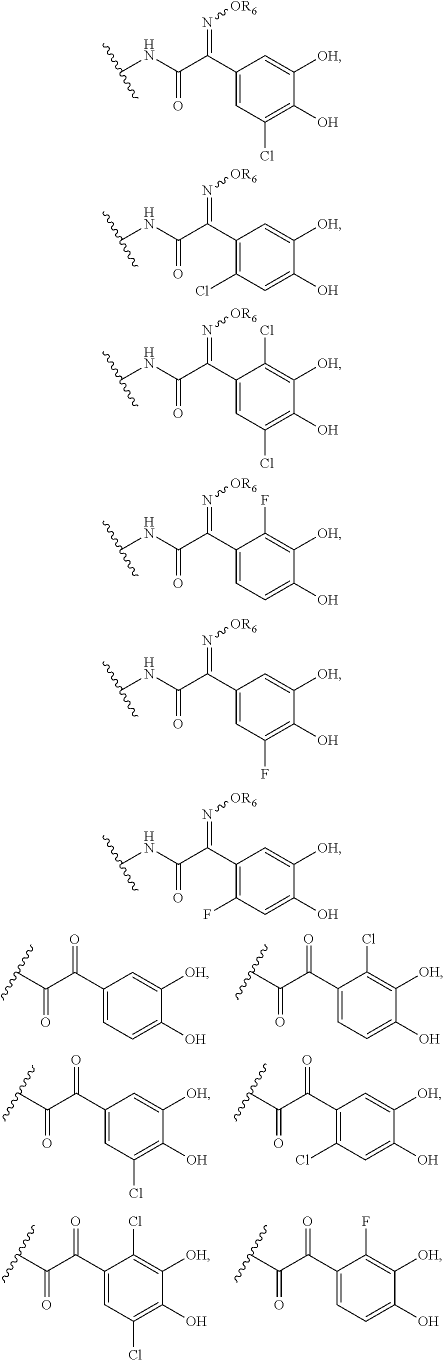 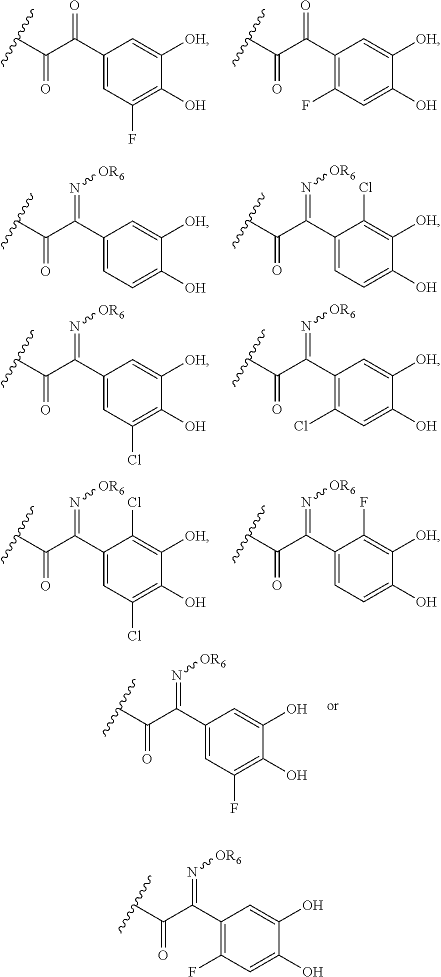
wherein
each R⁶ is independently a hydrogen atom, methyl, ethyl, 1-carboxy ethyl or 2-carboxy propane-2-yl,
the bond of wavy line means that the bond is cis-configuration, trans-configuration or the mixture thereof.

6. The compound, or an amino-protected compound when the amino is present on the ring in the 7-side chain, or a pharmaceutically acceptable salt thereof according to claim 1, wherein R¹⁰ is the above 2).

7. The compound, or an amino-protected compound when the amino is present on the ring in the 7-side chain, or a pharmaceutically acceptable salt thereof according to claim 6, wherein D-R¹⁰ is the group represented by Formula: 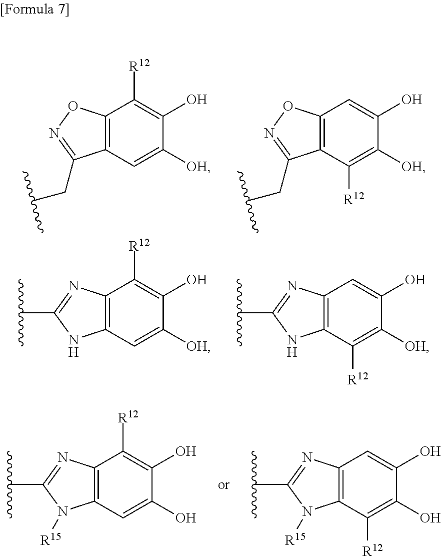
wherein
each R¹² is independently a hydrogen atom, halogen, hydroxy, —CN, —C(═O)—R¹⁵, —C(═O)—OH, —C(═O)—OR¹⁵ or OR¹⁵,
each R¹⁵ is independently lower alkyl or halo lower alkyl.

8. The compound, or an amino-protected compound when the amino is present on the ring in the 7-side chain, or a pharmaceutically acceptable salt thereof according to claim 6, wherein D-R¹⁰ is the group represented by Formula:

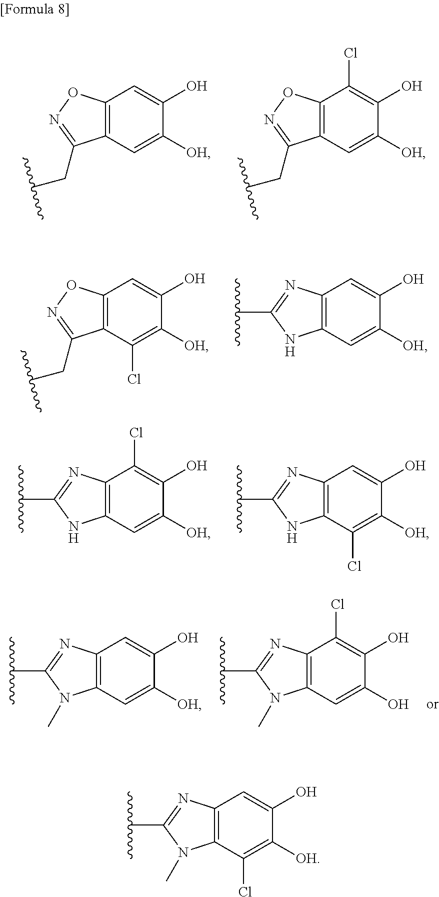

9. The compound, or an amino-protected compound when the amino is present on the ring in the 7-side chain, or a pharmaceutically acceptable salt thereof according to claim 1, wherein R¹⁰ is the above 3).

10. The compound, or an amino-protected compound when the amino is present on the ring in the 7-side chain, or a pharmaceutically acceptable salt thereof according to claim 1, wherein ring B is substituted or unsubstituted carbocycle.

11. The compound, or an amino-protected compound when the amino is present on the ring in the 7-side chain, or a pharmaceutically acceptable salt thereof according to claim 1, wherein Y is —C(═O)—.

12. The compound, or an amino-protected compound when the amino is present on the ring in the 7-side chain, or a pharmaceutically acceptable salt thereof according to claim 1, wherein
m is 1 or 2;
Q is —O—, —S—, —NR⁸—, —CR⁸R⁹—, —C(═O)— or —N═CH—, when m is 1;
each Q is independently —O—, —S—, —NR⁸—, or —C(═O)—, when m is 2.

13. The compound, or an amino-protected compound when the amino is present on the ring in the 7-side chain, or a pharmaceutically acceptable salt thereof according to claim 9, wherein D-R¹⁰ is the group represented by Formula: 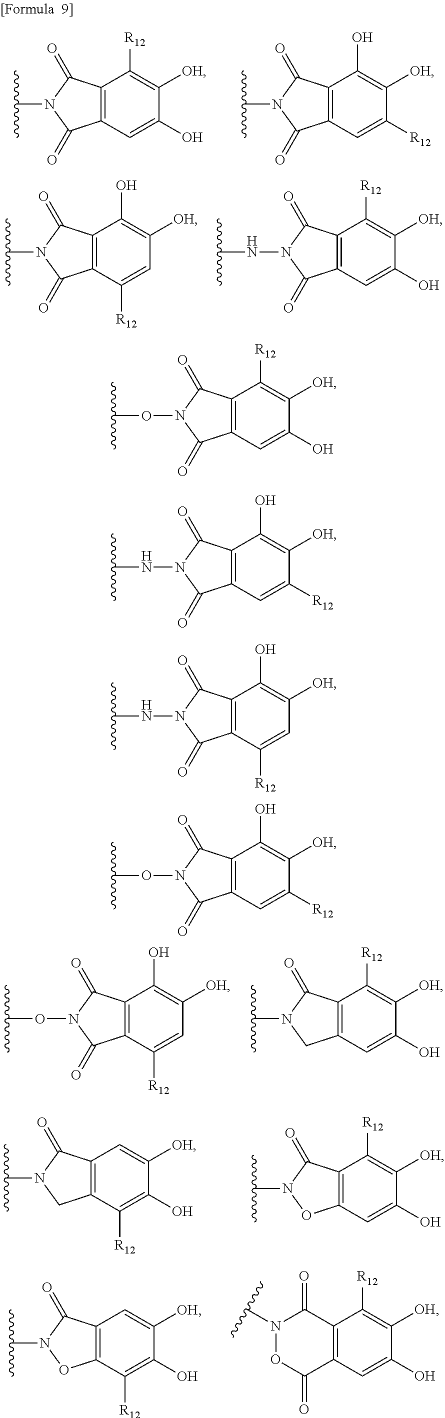 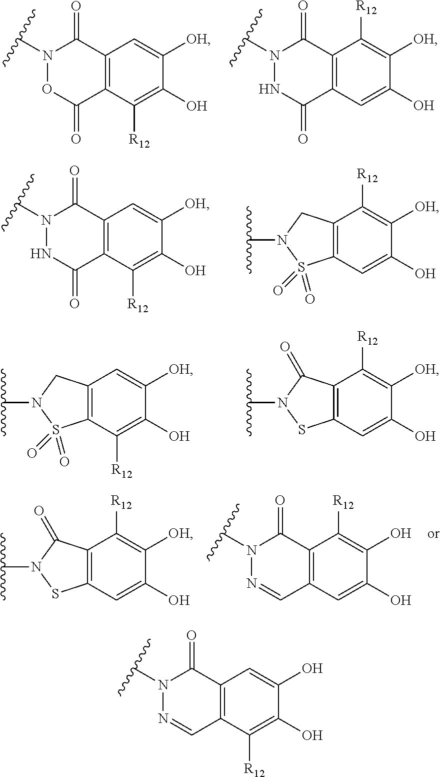
wherein
each R¹² is independently a hydrogen atom, halogen, hydroxy, —CN, —C(═O)—R¹⁵, —C(═O)—OH, —C(═O)—OR¹⁵ or OR¹⁵,
each R⁵ is independently lower alkyl or halo lower alkyl.

14. The compound, or an amino-protected compound when the amino is present on the ring in the 7-side chain, or a pharmaceutically acceptable salt thereof according to claim 9, wherein D-R¹⁰ is the group represented by Formula:

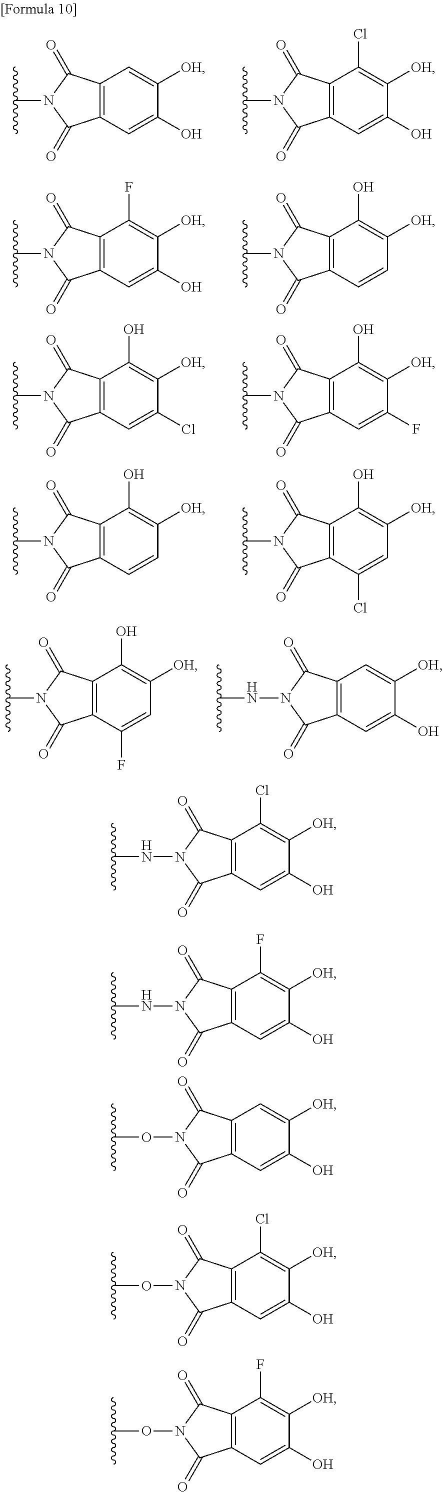

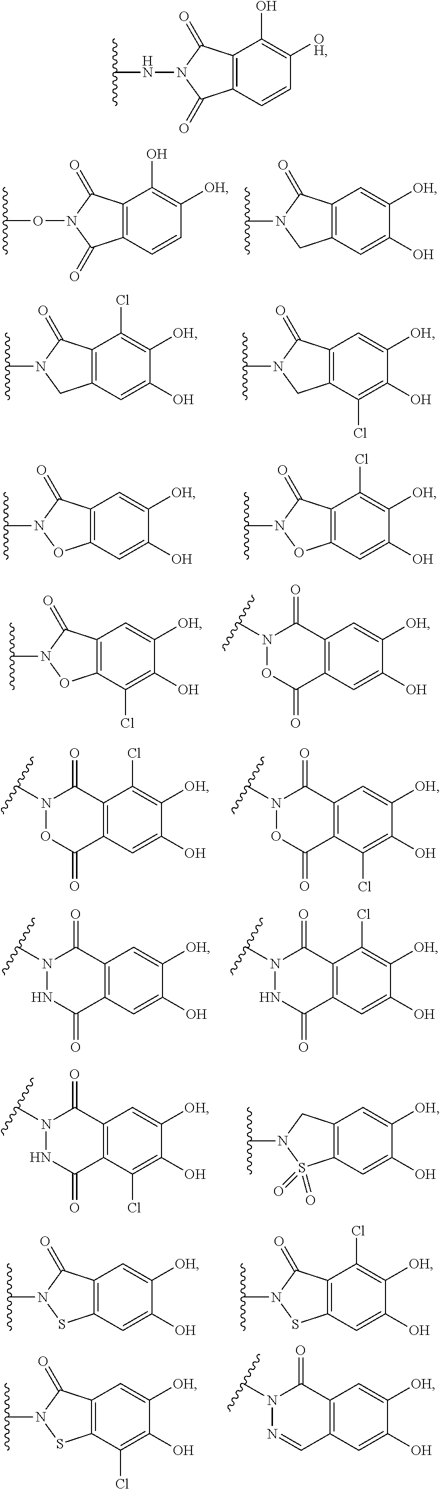

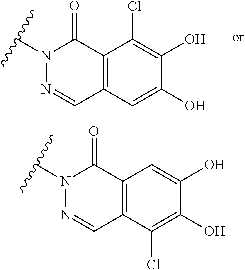

15. The compound, or an amino-protected compound when the amino is present on the ring in the 7-side chain, or a pharmaceutically acceptable salt thereof according to claim 1, wherein E is substituted or unsubstituted, saturated or unsaturated, monocyclic or polycyclic divalent group containing a quaternary ammonium ion represented by Formula: 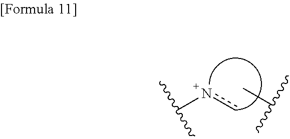
wherein
the dashed line is a bond in the ring,
the bond attached to the cationic nitrogen atom binds to L, and the other bond binds to G;
provided that,
when G binds to a cationic nitrogen atom, the dashed line is absent, and
when G does not bind to a cationic nitrogen atom, the dashed line is a single bond between the cationic nitrogen atom and a neighboring atom or an alkylene connecting the cationic nitrogen atom to a ring member atom other than said neighboring atom.

16. The compound, or an amino-protected compound when the amino is present on the ring in the 7-side chain, or a pharmaceutically acceptable salt thereof according to claim 1, wherein E is substituted or unsubstituted, saturated or unsaturated, monocyclic or polycyclic divalent group containing a quaternary ammonium ion represented by Formula: 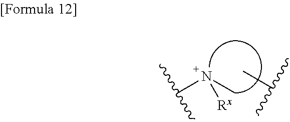
wherein
the bond attached to the cationic nitrogen atom binds to L, and the other bond binds to G;
R^{x} is substituted or unsubstituted lower alkyl.

17. The compound, or an amino-protected compound when the amino is present on the ring in the 7-side chain, or a pharmaceutically acceptable salt thereof according to claim 1, wherein E is a group selected from the following formulae which are optionally substituted on the ring: 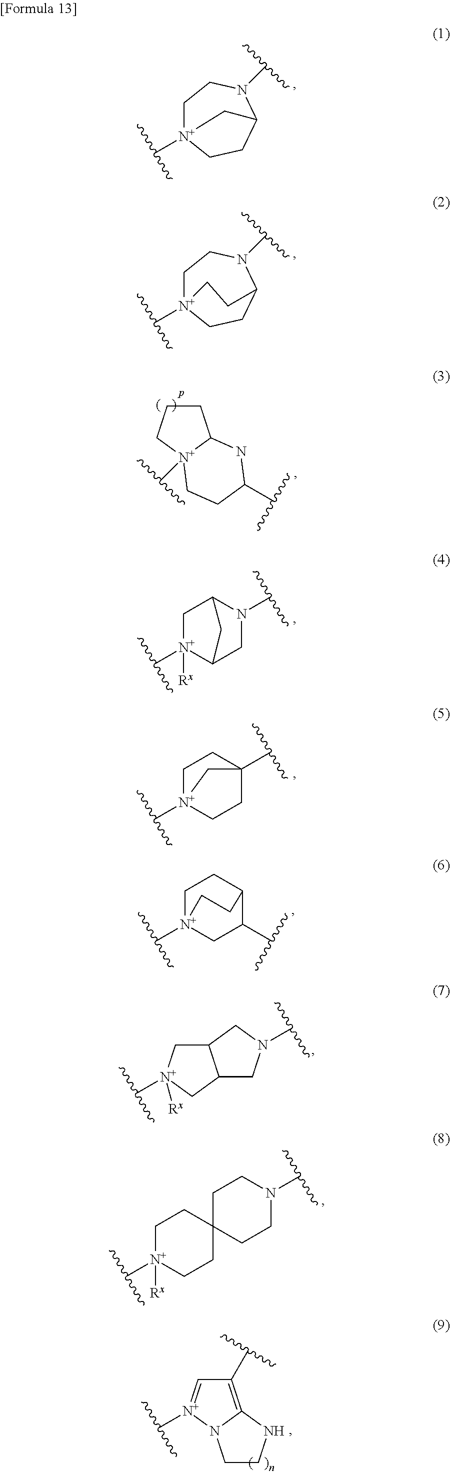 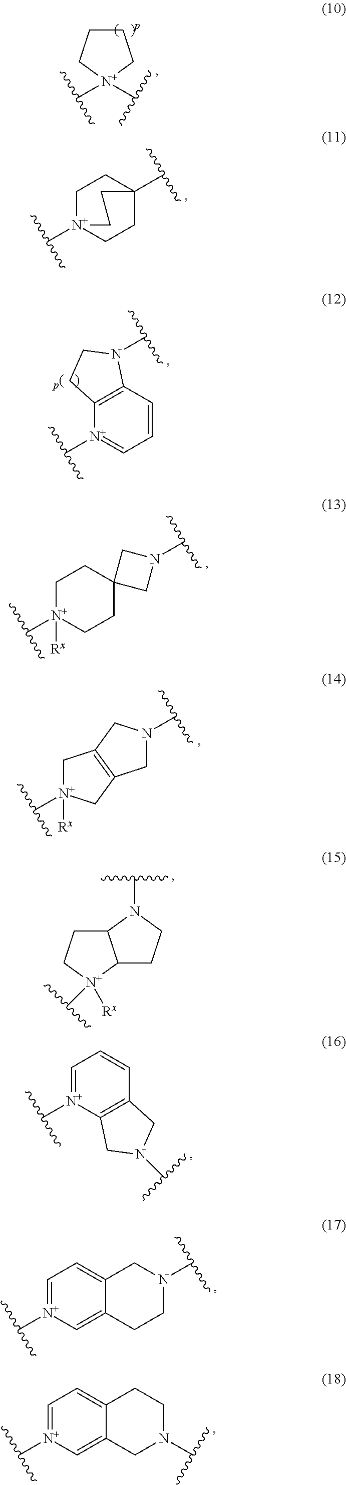 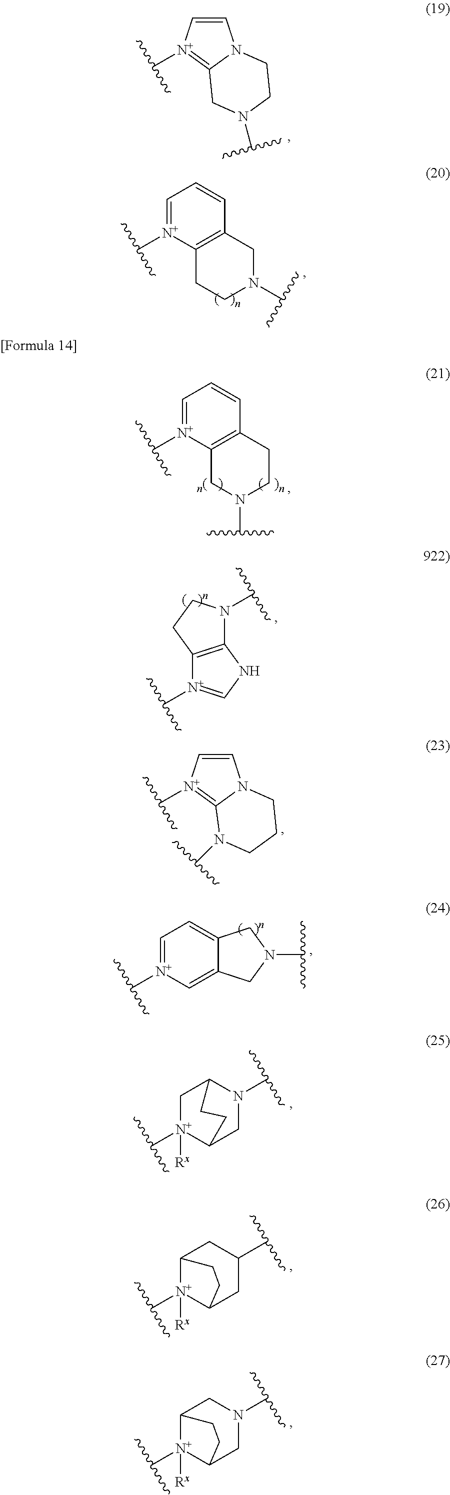 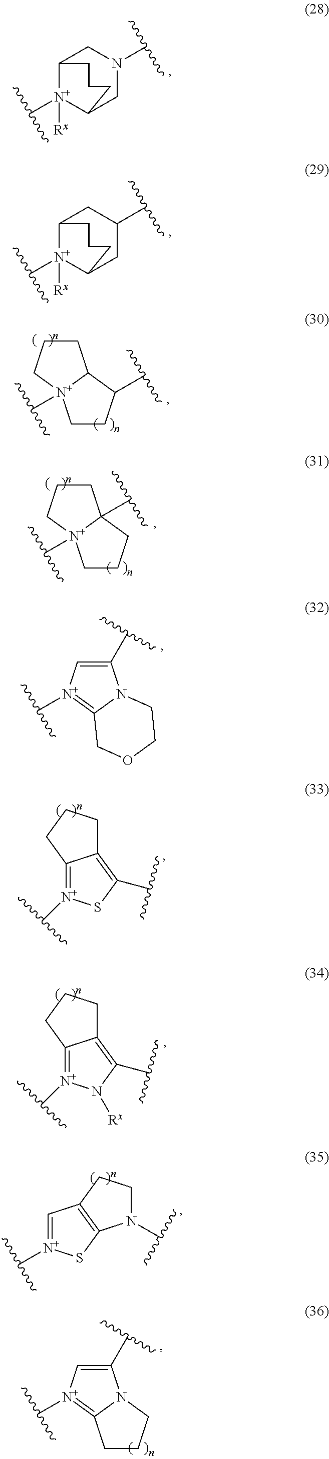 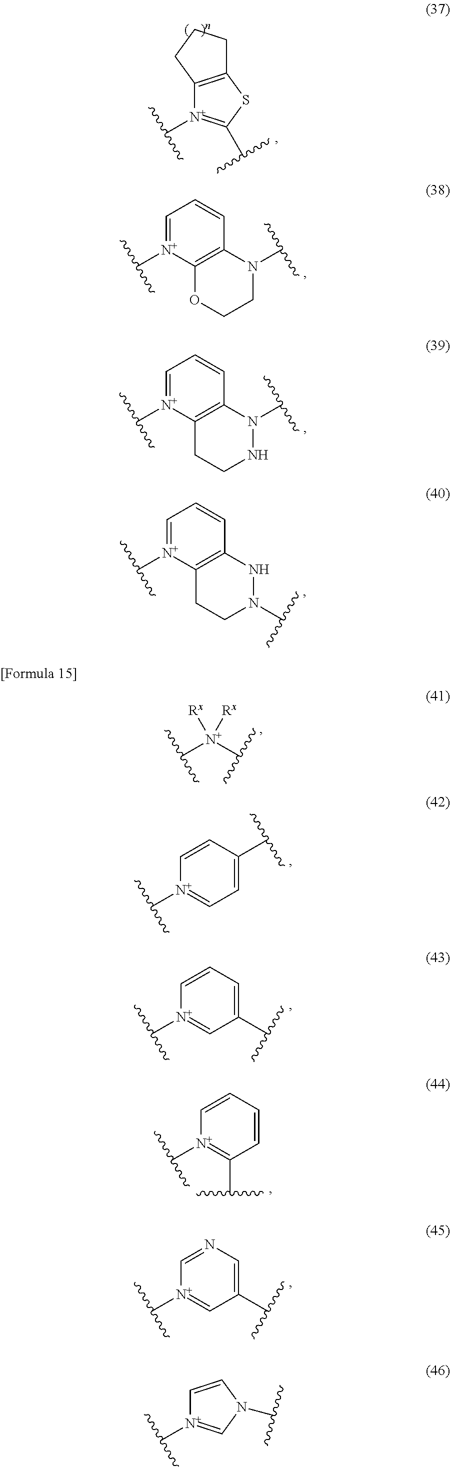 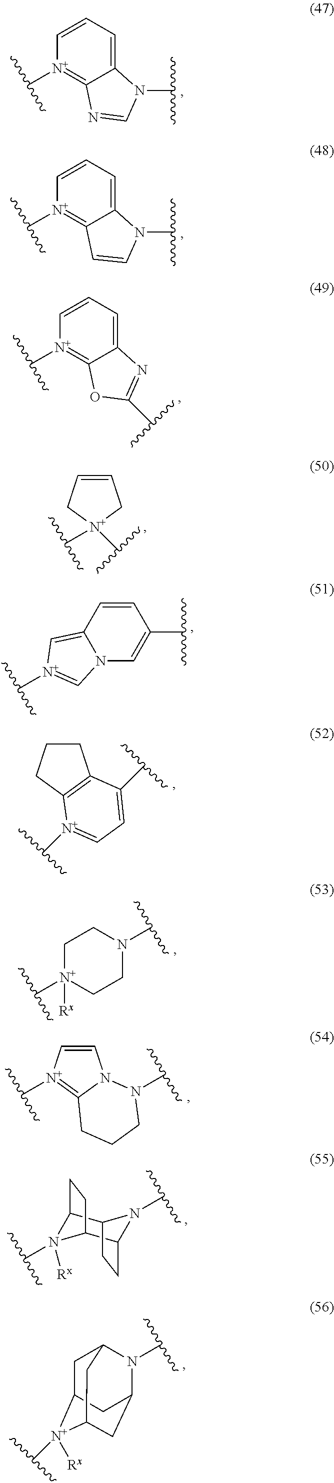 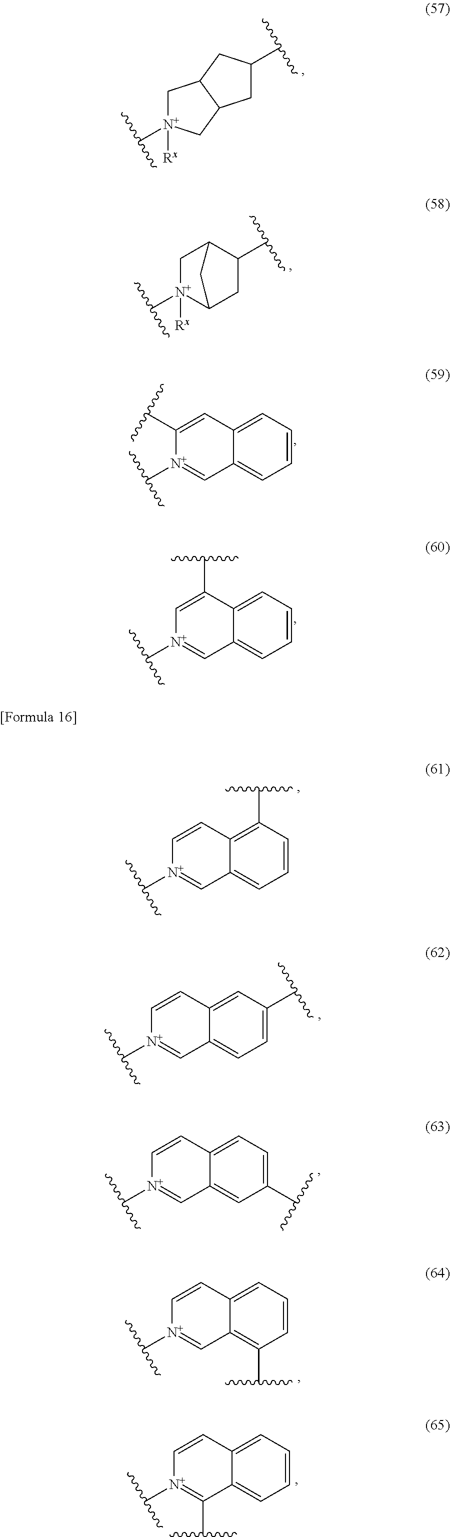 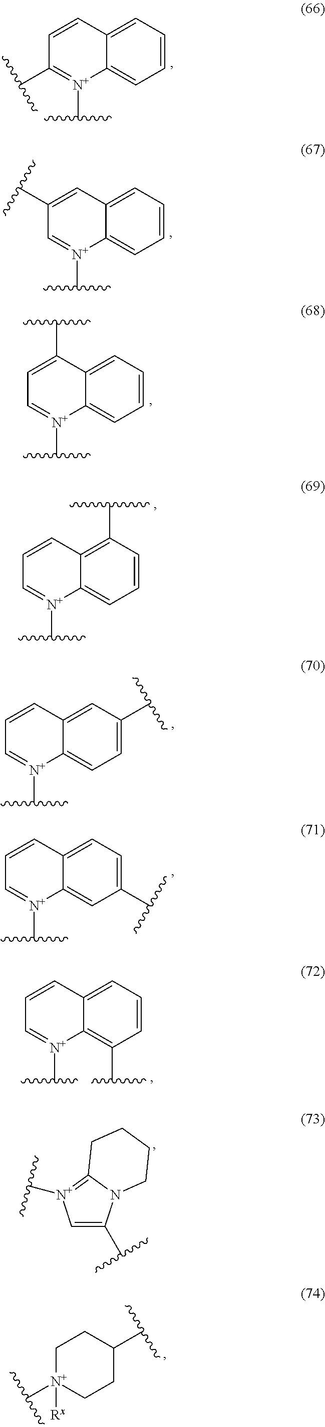 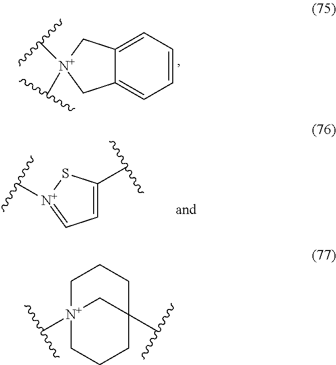 [SMILES: *[N+]1(*)Cc2ccccc2C1 ; *c1cc[n+](*)s1 ; *C12CCC[N+](*)(CCC1)C2]
wherein
the bond attached to the cationic nitrogen atom binds to L, and the other bond binds to G;
p is an integer from 1 to 3; n is 1 or 2; each R^{x} is independently substituted or unsubstituted lower alkyl.

18. The compound, or an amino-protected compound when the amino is present on the ring in the 7-side chain, or a pharmaceutically acceptable salt thereof according to claim 17, wherein E is a group selected from the group consisting of Formulae (1), (2), (5), (7), (10), (11), (26) to (29), (31) and (41).

19. The compound, or an amino-protected compound when the amino is present on the ring in the 7-side chain, or a pharmaceutically acceptable salt thereof according to claim 1, wherein the bioisostere of carboxylate anion (—COO⁻) is selected from —SO₃⁻, —S(═O)₂—N⁻—R¹³, —PO⁻—(OR¹³), —PO₂⁻—(OR¹³), —N⁻—C(═O)—R¹³, —C(═O)—N⁻—OR¹³, —C(═O)—NH—N⁻—S(═O)₂—R¹³, —C(═O)—N⁻—S(═O)₂—R¹³, —C(═O)—CH═C(O⁻)—R¹³, —N⁻—S(═O)₂—R¹³, —C(═O)—N⁻—S(═O)₂—R¹³, —N⁻—S(═O)₂—R¹³, —C(═O)—N⁻—C(═O)—R¹³, —C(═O)—N⁻—S(═O)₂—R¹³, —N⁻—C(═O)—R¹³, 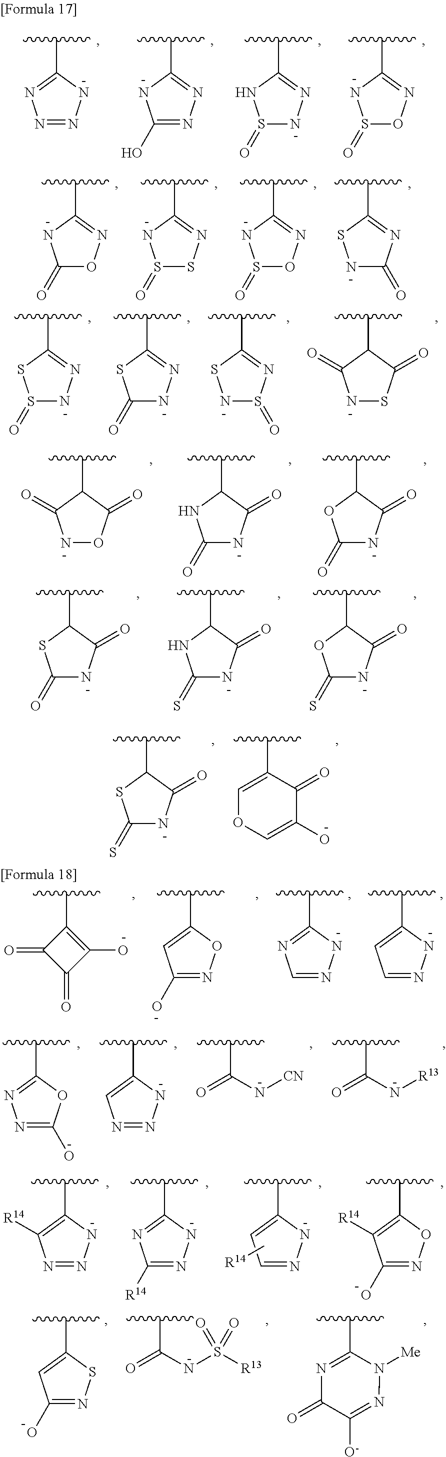 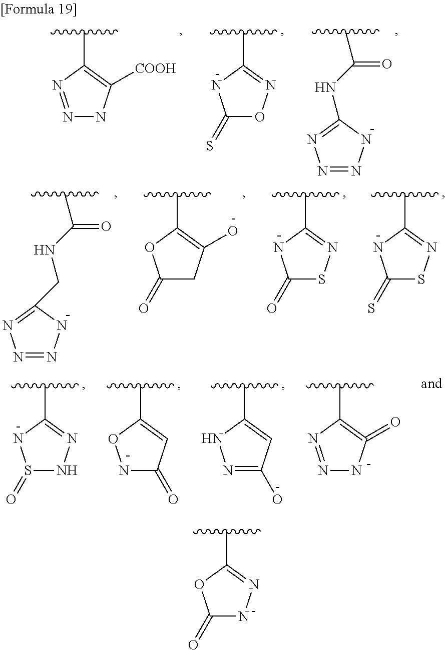
wherein
R¹³ is selected from the group consisting of hydrogen, hydroxy, halogen, substituted or unsubstituted lower alkyl, substituted or unsubstituted lower alkenyl, substituted or unsubstituted lower alkynyl, substituted or unsubstituted lower alkyloxy, substituted or unsubstituted amino, lower alkenyloxy, substituted or unsubstituted aryloxy, cyano, nitro, imino, mercapto, lower alkylthio, lower alkylsulfonyl, substituted or unsubstituted carbocyclyl, substituted or unsubstituted heterocyclyl and —CO₂R¹⁷ wherein R¹⁷ is hydrogen, lower alkyl or lower alkenyl; R¹⁴ is an electron-withdrawing group.

20. The compound, or an amino-protected compound when the amino is present on the ring in the 7-side chain, or a pharmaceutically acceptable salt thereof according to claim 19, wherein the bioisoster of carboxylate anion (—COO⁻) is the group represented by Formula:

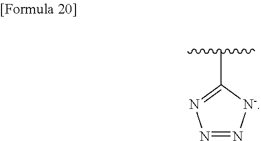

21. The compound, or an amino-protected compound when the amino is present on the ring in the 7-side chain, or a pharmaceutically acceptable salt thereof according to claim 1, wherein U is —S—.

22. The compound, or an amino-protected compound when the amino is present on the ring in the 7-side chain, or a pharmaceutically acceptable salt thereof according to claim 1, wherein W is —CH₂—.

23. The compound, or an amino-protected compound when the amino is present on the ring in the 7-side chain, or a pharmaceutically acceptable salt thereof according to claim 1, wherein R³ is a hydrogen atom or —OCH₃.

24. The compound, or an amino-protected compound when the amino is present on the ring in the 7-side chain, or a pharmaceutically acceptable salt thereof according to claim 1, wherein L is —CH₂—, —CH═CH—, —CH₂—CH═CH— or —CH═CH—CH₂—.

25. The compound, or an amino-protected compound when the amino is present on the ring in the 7-side chain, or a pharmaceutically acceptable salt thereof according to claim 1, wherein G is a single bond or substituted or unsubstituted lower alkylene.

26. The compound, or an amino-protected compound when the amino is present on the ring in the 7-side chain, or a pharmaceutically acceptable salt thereof according to claim 1, wherein R¹ is a group represented by Formula: 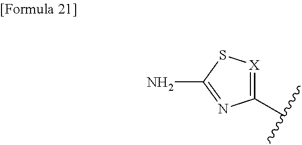
wherein X is N, C(—H) or C(—Cl).

27. The compound, or an amino-protected compound when the amino is present on the ring in the 7-side chain, or a pharmaceutically acceptable salt thereof according to claim 26, wherein X is N.

28. The compound, or an amino-protected compound when the amino is present on the ring in the 7-side chain, or a pharmaceutically acceptable salt thereof according to claim 26, wherein
X is C(—H) or C(—Cl).

29. The compound, or an amino-protected compound when the amino is present on the ring in the 7-side chain, or a pharmaceutically acceptable salt thereof according to claim 1, wherein R^{2A} and R^{2B} are taken together to form a substituted methylidene group shown below: 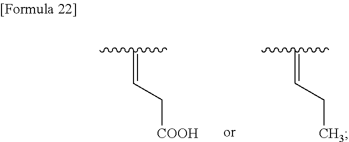
or
a substituted hydroxy imino group shown below: 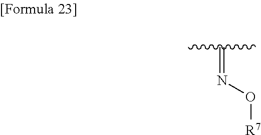
wherein R⁷ is substituted or unsubstituted lower alkyl group.

30. The compound, or an amino-protected compound when the amino is present on the ring in the 7-side chain, or a pharmaceutically acceptable salt thereof according to claim 1, wherein R^{2A} and R^{2B} are taken together to form a substituted hydroxyimino group shown below: 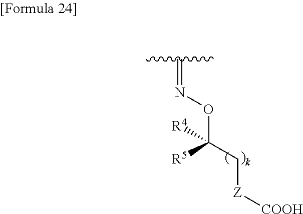
wherein
R⁴ and R⁵ are each independently a hydrogen atom, halogen, hydroxy, a carboxy group, a substituted or unsubstituted lower alkyl group, substituted or unsubstituted carbocyclyl, or substituted or unsubstituted heterocyclyl, or R⁴ and R⁵ may be taken together with a neighboring atom to form substituted or unsubstituted carbocycle or substituted or unsubstituted heterocycle;
Z is a single bond, optionally substituted carbocycle, or optionally substituted heterocycle;
k is an integer from 0 to 3.

31. The compound, or an amino-protected compound when the amino is present on the ring in the 7-side chain, or a pharmaceutically acceptable salt thereof according to claim 1, wherein R¹⁰ is
1) substituted phenyl or substituted 6-membered heterocyclyl containing 1 to 3 nitrogen atom(s),
2) substituted 9-membered bicyclic aromatic heterocyclyl, or
3) the group represented by the following formula: 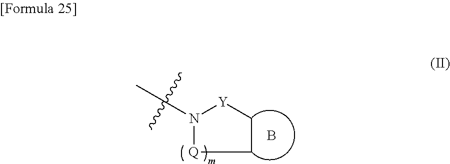
wherein
ring B is substituted carbocycle or substituted heterocycle, the other symbols are defined as above, and the substituents are at least two hydroxyl groups, and the each hydroxyl groups binds to the each adjacent carbon atom which is ring member atom.

32. A pharmaceutical composition, which comprises a compound or an amino-protected compound when the amino is present on the ring in the 7-side chain, or a pharmaceutically acceptable salt thereof according to claim 1.

33. A pharmaceutical composition, which comprises a compound or an amino-protected compound when the amino is present on the ring in the 7-side chain, or a pharmaceutically acceptable salt thereof according to claim 1, which possesses antimicrobial activity.

34. The pharmaceutical composition according to claim 32, which is for treating an infectious disease.

35. The compound, or an amino-protected compound when the amino is present on the ring in the 7-side chain, or a pharmaceutically acceptable salt thereof according to claim 1, which is for treating and/or preventing an infectious disease.

36. A method for treating and/or preventing an infectious disease, characterizes in the step of administering the compound, or an amino-protected compound when the amino is present on the ring in the 7-side chain, or a pharmaceutically acceptable salt thereof according to claim 1.
